(19) United States
(12) Patent Application Publication
(10) Pub. No.: US 2025/0297026 A1
(43) Pub. Date: Sep. 25, 2025

(54) THERAPEUTIC ANTIBODIES

(71) Applicant: PETMEDIX LTD., Cambridge (GB)

(72) Inventors: Allan BRADLEY, Cambridge (GB); Daniel BOLLAND, Cambridge (GB); Juexuan WANG, Cambridge (GB); Ruhcha Vijay SUTAVANI, Cambridge (GB)

(21) Appl. No.: 18/570,681

(22) PCT Filed: Jun. 17, 2022
(86) PCT No.: PCT/GB2022/051559

§ 371 Date: Dec. 15, 2023

(30) Foreign Application Priority Data
Jun. 17, 2021 (GB) 2108677.2
Feb. 25, 2022 (GB) 2202635.5

(51) Int. Cl. C07K 16/28; A61K 39/00; A61P 35/00

(52) U.S. Cl. CPC C07K 16/2887; A61P 35/00; A61K 2039/505; A61K 2039/55; C07K 2317/34; C07K 2317/41; C07K 2317/565; C07K 2317/732; C07K 2317/734; C07K 2317/92

(57) ABSTRACT

The invention relates to antibodies and antigen binding portions thereof that binds canine CD20. The present invention also relates to compositions and methods for the treatment of a condition mediated by B-cells in a canine subject.

### INTRODUCTION

Canine lymphomas are among the most common cancers diagnosed in dogs, representing around 7-14% of all cancers. As in humans, there are many different types of canine lymphoma and vary from rapidly progressing cancer to chronic disease.

CD20 is a cell-surface protein thought to be involved in regulation of B-cell proliferation and differentiation. The antigen comprises four transmembrane spanning regions and is present on the surface of almost all B-cells, both normal and malignant.

Human antibodies which recognise human CD20, such as rituximab, are used to treat human diseases characterized by excessive numbers of B-cells, or overactive or dysfunctional B-cells. These antibodies destroy B-cells. Rituximab is viewed as a revolutionary advance in the treatment of B-cell lymphoma.

Since the development of antibodies such as rituximab for humans over two decades ago, a number of antibodies which recognise canine CD20 have been reported in the literature. However, at this point in time, none are in common clinical use. There therefore still remains a need for different therapies. The invention is aimed at addressing this need.

### SUMMARY

In a first aspect, the invention relates to an isolated canine antibody or antigen-binding portion thereof which binds canine CD20 wherein said antibody comprises
a) a HC CDR1 sequence comprising or consisting of SEQ ID No. 27 or an amino acid sequence which has 1 or 2 amino acid differences compared to SEQ ID No. 27,
b) a HC CDR2 sequence comprising or consisting of SEQ ID No. 28 or an amino acid sequence which has 1, 2, 3 or 4 amino acid differences compared to SEQ ID No. 28,
c) a HC CDR3 sequence comprising or consisting of SEQ ID No. 29 or an amino acid sequence which has 1 or 2 amino acid differences compared to SEQ ID No. 29,
d) a LC CDR1 sequence comprising or consisting of SEQ ID No. 30 or an amino acid sequence which has 1 amino acid difference compared to SEQ ID No. 30,
e) a LC CDR2 sequence comprising or consisting of SEQ ID No. 31 or an amino acid sequence which has 1 amino acid difference compared to SEQ ID No. 31 and
f) a LC CDR3 sequence comprising or consisting of SEQ ID No. 32 or an amino acid sequence which has 1 or 2 amino acid differences compared to SEQ ID No. 32.

In another aspect of the invention or in an embodiment of the first aspect, the antibody or antigen-binding portion thereof has a HC CDR1 sequence comprising or consisting of SEQ ID No. 7, a HC CDR2 sequence comprising or consisting of SEQ ID No. 8, a HC CDR3 sequence comprising or consisting of SEQ ID No. 9, a LC CDR1 sequence comprising or consisting of SEQ ID No. 10, a LC CDR1 sequence comprising or consisting of SEQ ID No. 11 and a LC CDR1 sequence comprising or consisting of SEQ ID No. 12.

In another aspect of the invention or in an embodiment of the first aspect, the antibody has a HC CDR1 sequence comprising or consisting of SEQ ID No. 17, a HC CDR2 sequence comprising or consisting of SEQ ID No. 18, a HC CDR3 sequence comprising or consisting of SEQ ID No. 19, a LC CDR1 sequence comprising SEQ ID No. 20, a LC CDR2 sequence comprising or consisting of SEQ ID No. 21 and a LC CDR3 sequence comprising or consisting of SEQ ID No. 22.

In another aspect of the invention or in an embodiment of the first aspect, the antibody has a HC CDR1 sequence comprising SEQ ID No. 37, a HC CDR2 sequence comprising SEQ ID No. 38, a HC CDR3 sequence comprising SEQ ID No. 39, a LC CDR1 sequence comprising SEQ ID No. 40, a LC CDR2 sequence comprising SEQ ID No. 41 and a LC CDR3 sequence comprising SEQ ID No. 42.

In another aspect of the invention or in an embodiment of the first aspect, the antibody has a HC CDR1 sequence comprising or consisting of SEQ ID No. 47, a HC CDR2 sequence comprising or consisting of SEQ ID No. 48, a HC CDR3 sequence comprising or consisting of SEQ ID No. 49, a LC CDR1 sequence comprising or consisting of SEQ ID No. 50, a LC CDR2 sequence comprising or consisting of SEQ ID No. 51 and a LC CDR3 sequence comprising or consisting of SEQ ID No. 52.

In another aspect of the invention or in an embodiment of the first aspect, the antibody has a HC CDR1 sequence comprising or consisting of SEQ ID No. 82, a HC CDR2 sequence comprising or consisting of SEQ ID No. 83, a HC CDR3 sequence comprising or consisting of SEQ ID No. 84, a LC CDR1 sequence comprising or consisting of SEQ ID No. 85, a LC CDR2 sequence comprising or consisting of SEQ ID No. 86 and a LC CDR3 sequence comprising or consisting of SEQ ID No. 87.

In another aspect of the invention or in an embodiment of the first aspect, the antibody has a HC CDR1 sequence comprising or consisting of SEQ ID No. 92, a HC CDR2 sequence comprising or consisting of SEQ ID No. 93, a HC CDR3 sequence comprising or consisting of SEQ ID No. 94, a LC CDR1 sequence comprising or consisting of SEQ ID No. 95, a LC CDR2 sequence comprising or consisting of SEQ ID No. 96 and a LC CDR3 sequence comprising or consisting of SEQ ID No. 97.

In another aspect of the invention or in an embodiment of the first aspect, the antibody has a HC CDR1 sequence comprising or consisting of SEQ ID No. 102, a HC CDR2 sequence comprising or consisting of SEQ ID No. 103, a HC CDR3 sequence comprising or consisting of SEQ ID No. 104, a LC CDR1 sequence comprising or consisting of SEQ ID No. 105, a LC CDR2 sequence comprising or consisting of SEQ ID No. 106 and a LC CDR3 sequence comprising or consisting of SEQ ID No. 107.

In another aspect of the invention or in an embodiment of the first aspect, the antibody has a HC CDR1 sequence comprising or consisting of SEQ ID No. 112, a HC CDR2 sequence comprising or consisting of SEQ ID No. 113, a HC CDR3 sequence comprising or consisting of SEQ ID No. 114, a LC CDR1 sequence comprising or consisting of SEQ ID No. 115, a LC CDR2 sequence comprising or consisting of SEQ ID No. 116 and a LC CDR3 sequence comprising or consisting of SEQ ID No. 117.

In another aspect of the invention or in an embodiment of the first aspect, the antibody has a HC CDR1 sequence comprising or consisting of SEQ ID No. 122, a HC CDR2 sequence comprising or consisting of SEQ ID No. 123, a HC CDR3 sequence comprising or consisting of SEQ ID No. 124, a LC CDR1 sequence comprising or consisting of SEQ ID No. 125, a LC CDR2 sequence comprising or consisting of SEQ ID No. 126 and a LC CDR3 sequence comprising or consisting of SEQ ID No. 127.

In another aspect of the invention or in an embodiment of the first aspect, the antibody has a HC CDR1 sequence comprising or consisting of SEQ ID No. 132, a HC CDR2 sequence comprising or consisting of SEQ ID No. 133, a HC CDR3 sequence comprising or consisting of SEQ ID No. 134, a LC CDR1 sequence comprising or consisting of SEQ ID No. 135, a LC CDR2 sequence comprising or consisting of SEQ ID No. 136 and a LC CDR3 sequence comprising or consisting of SEQ ID No. 137.

In another aspect of the invention or in an embodiment of the first aspect, the antibody has a HC CDR1 sequence comprising or consisting of SEQ ID No. 142, a HC CDR2 sequence comprising or consisting of SEQ ID No. 143, a HC CDR3 sequence comprising or consisting of SEQ ID No. 144, a LC CDR1 sequence comprising or consisting of SEQ ID No. 145, a LC CDR2 sequence comprising or consisting of SEQ ID No. 146 and a LC CDR3 sequence comprising or consisting of SEQ ID No. 147.

In another aspect of the invention or in an embodiment of the first aspect, the antibody has a HC CDR1 sequence comprising or consisting of SEQ ID No. 152, a HC CDR2 sequence comprising or consisting of SEQ ID No. 153, a HC CDR3 sequence comprising or consisting of SEQ ID No. 154, a LC CDR1 sequence comprising or consisting of SEQ ID No. 155, a LC CDR2 sequence comprising or consisting of SEQ ID No. 156 and a LC CDR3 sequence comprising or consisting of SEQ ID No. 157.

In another aspect of the invention or in an embodiment of the first aspect, the antibody has a HC CDR1 sequence comprising or consisting of SEQ ID No. 162, a HC CDR2 sequence comprising or consisting of SEQ ID No. 163, a HC CDR3 sequence comprising or consisting of SEQ ID No. 164, a LC CDR1 sequence comprising or consisting of SEQ ID No. 165, a LC CDR2 sequence comprising or consisting of SEQ ID No. 166 and a LC CDR3 sequence comprising or consisting of SEQ ID No. 167.

In another aspect of the invention or in an embodiment of the first aspect, the antibody has a HC CDR1 sequence comprising or consisting of SEQ ID No. 172, a HC CDR2 sequence comprising or consisting of SEQ ID No. 173, a HC CDR3 sequence comprising or consisting of SEQ ID No. 174, a LC CDR1 sequence comprising or consisting of SEQ ID No. 175, a LC CDR2 sequence comprising or consisting of SEQ ID No. 176 and a LC CDR3 sequence comprising or consisting of SEQ ID No. 177.

In another aspect of the invention or in an embodiment of the first aspect, the antibody has a HC CDR1 sequence comprising or consisting of SEQ ID No. 182, a HC CDR2 sequence comprising or consisting of SEQ ID No. 183, a HC CDR3 sequence comprising or consisting of SEQ ID No. 184, a LC CDR1 sequence comprising or consisting of SEQ ID No. 185, a LC CDR2 sequence comprising or consisting of SEQ ID No. 186 and a LC CDR3 sequence comprising or consisting of SEQ ID No. 187.

In another aspect of the invention or in an embodiment of the first aspect, the antibody has a HC CDR1 sequence comprising or consisting of SEQ ID No. 192, a HC CDR2 sequence comprising or consisting of SEQ ID No. 193, a HC CDR3 sequence comprising or consisting of SEQ ID No. 194, a LC CDR1 sequence comprising or consisting of SEQ ID No. 195, a LC CDR2 sequence comprising or consisting of SEQ ID No. 196 and a LC CDR3 sequence comprising or consisting of SEQ ID No. 197.

In another aspect of the invention or in an embodiment of the first aspect, the antibody has a HC CDR1 sequence comprising or consisting of SEQ ID No. 202, a HC CDR2 sequence comprising or consisting of SEQ ID No. 203, a HC CDR3 sequence comprising or consisting of SEQ ID No. 204, a LC CDR1 sequence comprising or consisting of SEQ ID No. 205, a LC CDR2 sequence comprising or consisting of SEQ ID No. 206 and a LC CDR3 sequence comprising or consisting of SEQ ID No. 207.

In another aspect of the invention or in an embodiment of the first aspect, the wherein said antibody has a HC CDR1 sequence comprising or consisting of SEQ ID No. 212, a HC CDR2 sequence comprising or consisting of SEQ ID No. 213, a HC CDR3 sequence comprising or consisting of SEQ ID No. 214, a LC CDR1 sequence comprising or consisting of SEQ ID No. 215, a LC CDR2 sequence comprising or consisting of SEQ ID No. 216 and a LC CDR3 sequence comprising or consisting of SEQ ID No. 217.

In another aspect of the invention or in an embodiment of the first aspect, the antibody has a HC CDR1 sequence comprising or consisting of SEQ ID No. 222, a HC CDR2 sequence comprising or consisting of SEQ ID No. 223, a HC CDR3 sequence comprising or consisting of SEQ ID No. 224, a LC CDR1 sequence comprising or consisting of SEQ ID No. 225, a LC CDR2 sequence comprising or consisting of SEQ ID No. 226 and a LC CDR3 sequence comprising or consisting of SEQ ID No. 227.

In another aspect of the invention or in an embodiment of the first aspect, the antibody has a HC CDR1 sequence comprising or consisting of SEQ ID No. 232, a HC CDR2 sequence comprising or consisting of SEQ ID No. 233, a HC CDR3 sequence comprising or consisting of SEQ ID No. 234, a LC CDR1 sequence comprising or consisting of SEQ ID No. 235, a LC CDR2 sequence comprising or consisting of SEQ ID No. 236 and a LC CDR3 sequence comprising or consisting of SEQ ID No. 237.

In one embodiment, the antibody or antigen-binding portion thereof comprises a HC variable region sequence comprising SEQ ID NO. 24 or a sequence with at least 75%, 80%, 85% or 90% sequence identity thereto and a LC variable region sequence comprising SEQ ID NO. 26 or a sequence with at least 70%, 75%, 80%, 85%, 90% or 95% sequence identity thereto.

For example, the antibody or antigen-binding portion thereof has
a) a HC variable region sequence comprising SEQ ID NO. 4 and a LC variable region sequence comprising SEQ ID NO. 6;
b) a HC variable region sequence comprising SEQ ID NO. 14 and a LC variable region sequence comprising SEQ ID NO. 16;
c) a HC variable region sequence comprising SEQ ID NO. 34 and a LC variable region sequence comprising SEQ ID NO. 36;
d) a HC variable region sequence comprising SEQ ID NO. 44 and a LC variable region sequence comprising SEQ ID NO. 46;
e) a HC variable region sequence comprising SEQ ID NO. 79 and a LC variable region sequence comprising SEQ ID NO. 81 or a LC variable region sequence comprising SEQ ID NO. 238;
f) a HC variable region sequence comprising SEQ ID NO. 89 and a LC variable region sequence comprising SEQ ID NO. 91 or a LC variable region sequence comprising SEQ ID NO. 239;
g) a HC variable region sequence comprising SEQ ID NO. 99 and a LC variable region sequence comprising SEQ ID NO. 101 or a LC variable region sequence comprising SEQ ID NO. 240;
h) a HC variable region sequence comprising SEQ ID NO. 109 and a LC variable region sequence comprising SEQ ID NO. 111 or a LC variable region sequence comprising SEQ ID NO. 241;
i) a HC variable region sequence comprising SEQ ID NO. 119 and a LC variable region sequence comprising SEQ ID NO. 121;
j) a HC variable region sequence comprising SEQ ID NO. 129 and a LC variable region sequence comprising SEQ ID NO. 131 or a LC variable region sequence comprising SEQ ID NO. 242;
k) a HC variable region sequence comprising SEQ ID NO. 139 and a LC variable region sequence comprising SEQ ID NO. 141 or a LC variable region sequence comprising SEQ ID NO. 243;
I) a HC variable region sequence comprising SEQ ID NO. 149 and a LC variable region sequence comprising SEQ ID NO. 151 or a LC variable region sequence comprising SEQ ID NO. 244;
m) a HC variable region sequence comprising SEQ ID NO. 159 and a LC variable region sequence comprising SEQ ID NO. 161 or a LC variable region sequence comprising SEQ ID NO. 245;
n) a HC variable region sequence comprising SEQ ID NO. 169 and a LC variable region sequence comprising SEQ ID NO. 171;
o) a HC variable region sequence comprising SEQ ID NO. 179 and a LC variable region sequence comprising SEQ ID NO. 181 or a LC variable region sequence comprising SEQ ID NO. 246;
p) a HC variable region sequence comprising SEQ ID NO. 189 and a LC variable region sequence comprising SEQ ID NO. 191 or a LC variable region sequence comprising SEQ ID NO. 247;
q) a HC variable region sequence comprising SEQ ID NO. 199 and a LC variable region sequence comprising SEQ ID NO. 201 a LC variable region sequence comprising SEQ ID NO. 248;
r) a HC variable region sequence comprising SEQ ID NO. 209 and a LC variable region sequence comprising SEQ ID NO. 211 a LC variable region sequence comprising SEQ ID NO. 249;
s) a HC variable region sequence comprising SEQ ID NO. 219 and a LC variable region sequence comprising SEQ ID NO. 221; or
t) a HC variable region sequence comprising SEQ ID NO. 229 and a LC variable region sequence comprising SEQ ID NO. 231 a LC variable region sequence comprising SEQ ID NO. 250.

For example, the antigen-binding portion thereof is an scFv, Fv, heavy chain or single domain antibody.

The invention also relates to an isolated canine antibody or antigen-binding portion thereof that binds to canine CD20 competing with an antibody or antigen-binding portion thereof as described above.

For example, the antibody or antigen-binding portion thereof is conjugated to a therapeutic moiety.

For example, the therapeutic moiety is a second antibody or antigen-binding portion thereof.

For example, the second antibody or antigen-binding portion thereof binds to a different target.

For example, the antibody or antigen-binding portion thereof is conjugated to a further moiety selected from a half life extending moiety, label, cytotoxin, liposome, nanoparticle or radioisotope.

For example, the said antibody or antigen-binding portion thereof is a-fucosylated.

The invention also relates to a pharmaceutical composition comprising an antibody or antigen-binding portion thereof as described above.

The invention also relates to a method of treating a condition mediated by B-cells in a canine subject in need thereof comprising administering an effective amount of the antibody or antigen-binding portion thereof as described above or a pharmaceutical composition as described above.

For example, the condition mediated by B-cells is a B cell lymphoma or leukaemia.

For example, the condition mediated by B-cells is an immune mediated disease.

For example, the immune mediated disease is an autoimmune disease.

For example, another therapeutic agent can be separately administered to the subject.

For example, the therapeutic agent is a cytotoxic agent or a radiotoxic agent.

For example, the therapeutic agent is an immunosuppressant.

For example, the therapeutic agent is an immunological modulating agent, such as a cytokine or a chemokine.

The invention also relates to the antibody or antigen-binding portion as described above or the pharmaceutical composition as described above for use in the treatment of disease.

For example, in the disease is a disease mediated by B-cells.

For example, the disease mediated by B-cells is a B cell lymphoma or leukaemia.

For example, the disease mediated by B-cells is an immune mediated disease.

For example, the immune mediated disease is an autoimmune disease.

The invention also relates to a method of inhibiting tumor growth or metastasis comprising contacting a tumor cell with an effective amount of the antibody or antigen-binding portion thereof as described above or pharmaceutical composition as described above.

The invention also relates to a method of killing a tumor cell expressing CD20, comprising contacting the cell with the antibody of as described above or a pharmaceutical composition as described above, such that killing of the cell expressing CD20 occurs.

For example, the tumor cell is a canine tumor cell.

The invention also relates to a nucleic acid sequence that encodes an antibody or antibody antigen-binding portion thereof as described above.

For example, the nucleic acid sequence comprises a sequence selected from SEQ ID NOs. 3, 5, 13, 15, 23, 25, 33, 35, 43, 45, 78, 80, 88, 90, 98, 100, 108, 110, 118, 120, 128, 130, 138, 140, 148, 150, 158, 160, 168, 170, 178, 180, 188, 190, 198, 200, 208, 210, 218, 220, 228, 230, 251, 252, 253, 254, 255, 256, 257, 258, 259, 260, 261, 262, 263, 264, 265, 266, 267, 268, 269, 270,271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or 301.

The invention also relates to a vector comprising a nucleic acid sequence as described above.

The invention also relates to a host cell comprising the nucleic acid sequence as described above or a vector of as described above.

The invention also relates to a kit comprising an antibody or antigen-binding portion thereof as described above or a pharmaceutical composition as described above.

For example, further comprises a reagent for the detection of the antibody or antigen-binding portion thereof.

The invention also relates to a method for making a canine antibody that binds CD20 comprising culturing the isolated host cell as described above and recovering said antibody.

The invention also relates to a method for making a canine antibody that binds CD20 comprising the steps of
a) immunising a transgenic mouse that expresses a nucleic acid construct comprising canine heavy chain V genes and canine light chain V genes with CD20 antigen,
b) generating a library of antibodies from said mouse and
c) isolating an antibody from said library.

The invention also relates to a method for detecting a CD20 protein or an extracellular domain of a CD20 protein in a biological sample from a canine subject, comprising contacting a biological sample with the antibody or antigen-binding portion thereof as described above wherein said antibody or antigen-binding portion thereof is linked to a detectable label.

For example, the biological sample is a biopsy, tissue, blood, serum, plasma, or lymphatic fluid sample.

The invention also relates to an antibody or antigen binding portion thereof that binds canine at an epitope comprises one or more amino residues e.g.: 1 to 10, e.g.: 1, 2, 3, 4, 5, 6, 7, 8, 9 or 10, of the following amino residues or consists of the following amino residues ENLNLIKAPM (SEQ ID NO. 303, amino acid No. 150-159 in Seq. ID NO: 2).

### FIGURES

The invention is further described in the following non-limiting figures.

FIG. 1. Antibody titre of canine CD20 immunised Ky9 mice. Post-immunisation sera were serially diluted (as shown) and added to canine CD20-expressing cells. Subsequently, the cells were incubated with fluorophore-conjugated anti-mouse IgG1, IgG2a, IgG2b secondary antibodies and the samples were assessed on a flow cytometer. Binding of pre-immunisation sera to canine CD20-expressing cells is presented as the background line in the plot.

FIG. 2. (A) Amino acid sequence alignment of variable regions of PMX001, PMX002, PMX003, PMX004 and PMX005 mAbs. The shading indicates the CDR1, CDR2 and CDR3 regions in both heavy chain (HC) and light chain (LC) sequences of the mAbs. (B) Amino acid sequence alignment of HC and LC variable regions of the mAbs. The boxes indicate the CDR1, CDR2 and CDR3 regions in both heavy chain (HC) and light chain (LC) sequences of the mAbs.

FIG. 3. Binding assessment of anti-canine CD20 mAbs using flow cytometry. PMX001, PMX002, PMX003, PMX004, PMX005, 1 E4-cIGGB, 4E1-7-clGGB (see A) and PMX006, PMX007, PMX008, PMX009, PMX010, PMX011 (see B) and PMX003, PMX066, PMX067, PMX069, PMX070, PMX071, PMX072, PMX073, PMX074, PMX075, PMX076, PMX077, PMX078, PMX079, PMX080, PMX081, 1E4-cIGGB, 4E1-7-clGGB (see C) were applied to cCD20-expressing MDCK II cells at 10 μg/ml. Rituximab-clGGB (grey shadow) was used as the isotype control and presented in each plot. (D) Affinity determination of PMX003, PMX115 and PMX070 mAbs. Binding of 1:2 serially diluted antibodies to cCD20-expressing MDCK II cells was assessed in a binding assay and the mean values of the fluorophore signal was determined. The plot of mean fluorescence intensity (MFI) versus concentration was used to determine EC50 of binding, using non-linear regression, which is the apparent Kd of antibody binding to cell surface expressed canine CD20.

FIG. 4. CDC activity of anti-canine CD20 antibodies. Both native and a-fucosylated format of antibodies were analysed at a range of concentrations (as shown) in the CDC assay. Rituximab-clGGB chimeric antibody was used as the “isotype control”. “F” refers to the a-fucosylated format of the antibodies.

FIG. 5. ADCC activity of anti-canine CD20 antibodies. Both native and a-fucosylated format of antibodies were analysed at a range of concentrations (as shown) in the ADCC assay. Rituximab-clGGB chimeric antibody was used as the “isotype contro”l. “F” refers to the a-fucosylated format of the antibodies. EC50 values are also presented for each antibody.

FIG. 6. B cell depletion efficiency of anti-canine CD20 mAbs in whole canine blood. Freshly drawn dog whole blood was diluted with culture media and incubated with 10 μg/ml of PMX001, PMX003, 1E4-cIGGB, 4E1-7-clGGB (see A), native or a-fucosylated format of PMX003 and 4E1-7-clGGB antibodies, (see B), PMX003, PMX006, PMX007, PMX008, PMX009, PMX010, PMX011 (see C), or no antibody, for 24 h. Rituximab-clGGB was used as the “isotype control” (Iso Ctrl). “F” refers to the a-fucosylated format of the antibodies.

FIG. 7. The B cell depletion efficiency of anti-canine CD20 mAbs in healthy Beagles. (A-B) Three healthy beagles for each test group were given the following antibodies by intravenous administration: Isotype control (rituximab-clgGB, 2.5 mg/kg), low dose of PMX003 (0.5 mg/kg), high dose of PMX003 (2.5 mg/kg) and low dose of a-fucosyslated PMX003 (0.5 mg/kg). “F” refers to a-fucosylated format of antibodies. The percentage of CD21+ B cells (A) and CD8+ T cells (B) in lymphocytes was analysed using flow cytometry on day 0, 1, 2, 5, 7, 15, 27 and 43. The data shown here is representative of four technical repeats. (C) Three healthy beagles for each test group were given the following antibodies by intravenous administration: Isotype control (rituximab-clgGB, 2 mg/kg), low dose of PMX115 (0.5 mg/kg), high dose of PMX115 (2 mg/kg), low dose of PMX070 (0.5 mg/kg) and high dose of PMX070 (2 mg/kg). The percentage of CD21+ B cells in lymphocytes was analysed using flow cytometry on day 0, 1, 4, 7, 14, 21 and 28. The data shown here is representative of four technical repeats.

FIG. 8. Epitope mapping strategy for anti-CD20 antibodies. (A) The canine CD20 extracellular domain sequence was mutated to the human CD20 sequence at equivalent locations in vectors DH01-DH09. (B) The canine CD20 extracellular domain sequence was mutated to the murine CD20 sequence at equivalent locations in vectors DM01-DM09. (C) Every residue in the small loop and every other residue in the large loop of canine CD20 extracellular domain was mutated to alanine, giving rise to vectors S01-S09 and L01-L25. Mutated residues are highlighted in bold.

FIG. 9. Binding of PMX003, PMX115 and PMX070 mAbs to MDCK cells expressing dog-human chimeric CD20 or dog-mouse chimeric CD20 was evaluated by flow cytometry. Black bars of 01-09 in the figure represent binding to DH01-DH09 sequences shown in FIG. 8A and grey bars of 01-09 in the figure represent binding to DM01-DM09 sequences shown in FIG. 8B. MDCK cells expressing wildtype canine CD20 were applied as positive controls and wildtype MDCK cells were used as negative controls.

Table 1. Amino Acid Residues and Examples of Conservative Amino Acid Substitutions
Table 2. Sequences
Table 3. VH and VL gene usage
Table 4. Summary of functional data for antibodies
Table 5. Developability profile for antibodies

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The inventors have developed fully canine antibodies that bind specifically to canine CD20. These antibodies were generated in transgenic rodents expressing canine V, D, J genes. Therefore, the antibodies are less likely to be immunogenic for administration to canine subjects than caninized antibodies or chimeric antibodies. Furthermore, as these antibodies can be used directly, with no further modifications to their variable regions, there is no risk of reducing the affinity or otherwise compromising the antibody. Other technologies risk introducing development or efficacy liabilities through the ex vivo combination of antibody sequences of canine origin with that from another species, typically rodent. Thus, the invention relates to an isolated canine antibody or antigen-binding portion thereof which binds canine CD20.

The properties of the antibodies and antigen binding portions thereof of the invention can be exploited in therapeutic methods and uses as well as in pharmaceutical formulations as described herein.

The term CD20 refers to the B-lymphocyte antigen CD20. The antibodies and antigen binding portions thereof bind specifically to wild type canine CD20 as defined in SEQ ID No. 1 (nucleotide sequence) and SEQ ID No. 2 (amino acid sequence). Unless otherwise stated, the term CD20 as used herein refers to canine CD20. B-lymphocyte antigen CD20 or CD20 is expressed on the surface of all B-cells beginning at the pro-B phase (CD45R+, CD117+) and progressively increasing in concentration until maturity. In humans and canines, CD20 is encoded by the MS4A1 gene.

The terms “CD20 binding molecule/protein/polypeptide/agent/moiety”, “CD20 antigen binding molecule molecule/protein/polypeptide/agent/moiety”, “anti-CD20 antibody”, “anti-CD20 antibody or antigen binding portion thereof” all refer to a molecule capable of specifically binding to the canine CD20 antigen. The binding reaction may be shown by standard methods, for example with reference to a negative control test using an antibody of unrelated specificity.

An antibody or antigen binding portion thereof of the invention, including a multispecific, e.g. bispecific or trispecific, binding agent described herein, “which binds” or is “capable of binding” an antigen of interest, that is canine CD20, is one that binds the antigen with sufficient affinity such that the antibody or antigen binding portion thereof is useful as a therapeutic agent in targeting a cell or tissue expressing the antigen CD20 as described herein.

The antibody or antigen binding portion thereof according to the invention bind specifically to canine CD20. In other words, binding to the CD20 antigen is measurably different from a non-specific interaction. In particular, the antibodies described herein do not cross react with mouse CD20.

The term “specific binding” or “specifically binds to” or is “specific for” a particular polypeptide or an epitope on a particular polypeptide target as used herein can be exhibited, for example, by a molecule having a KD for the target of at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or lower. In one embodiment, the KD is at least about 10⁻⁸ M to about 10⁻⁹ M, e.g. In one embodiment, the KD is in the nanomolar range. In one embodiment, the term “specific binding” refers to binding where a molecule binds to a particular polypeptide or epitope on a particular polypeptide without substantially binding to any other polypeptide or polypeptide epitope. The terms KD and KD are used interchangeably herein.

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, C_{L}.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can generally be of any isotype, class or subclass. The C_{H}3 domain according to the various aspects of the invention is a C_{H}3 domain of the canine IgG subtype, for example IgG-A, IgG-B, IgG-C, and IgG-D.

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). The amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). The antibodies herein may have a lambda or kappa light chain. In one embodiment, the light chain is a lambda light chain.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005). The IMGT numbering scheme is used herein unless otherwise specified.

A chimeric antibody is a recombinant protein that contains the variable domains including the complementarity determining regions (CDRs) of an antibody derived from one species, preferably a rodent or human antibody, while the constant domains of the antibody molecule are derived from those of a canine antibody.

As used herein, the term “caninized antibody” refers to forms of recombinant antibodies that contain sequences from both canine and non-canine (e.g., murine) antibodies. In general, a caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target. The non-canine sequences, e.g., of the hypervariable loops, may further be compared to canine sequences and as many residues changed to be as similar to authentic canine sequences as possible.

In contrast, fully canine antibodies of the present invention have canine variable regions and do not include full or partial CDRs or FRs from another species. Advantageously, fully canine antibodies as described herein have been obtained from transgenic mice comprising canine immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine antibodies produced in this way generate antibodies with optimal properties for developability, minimizing lengthy lead optimization prior to production at scale. Advantageously, such fully canine antibodies present the lowest possible risk of immunogenicity when introduced into a patient animal which, in turn, facilitates a repeated dosing regime. Given that ex vivo mAb engineering runs the risk of introducing development liabilities, immunogenicity, and reduced affinity (as outlined above), fully canine antibodies of the present invention are, therefore, most likely to be efficacious therapies in a clinical context. Thus, in an embodiment, the term canine antibody refers to a fully canine antibody.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations, carbohydrate addition) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The term “antigen binding site” refers to the part of the antibody or antibody fragment that comprises the area that specifically binds to an antigen. An antigen binding site may be provided by one or more antibody variable domains. An antigen binding site is typically comprised within the associated V_{H} and V_{L} of an antibody or antibody fragment.

The term “epitope” or “antigenic determinant” refers to a site on the surface of an antigen to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping by alanine-scanning mutagenesis or Pepscan) are well known in the art and include, for example, immunoblotting and immunoprecipitation assays, wherein overlapping or contiguous peptides are tested for reactivity with a given antibody or antibody fragment. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody.

In one embodiment, the antibody or antigen binding portion thereof as described herein binds an epitope that comprises one or more amino residues, e.g. 1 to 19, e.g. 1, 2, 3, 4, 5, 6, 7, 8, 9, 10, 11, 12, 13, 14, 15, 16, 17, 18 or 19, of the following amino residues or consists of the following amino residues ITISHFFKMENLNLIKAPM (SEQ ID NO. 302, amino acid No. 141-159 in Seq ID NO. 2). In one embodiment, the epitope comprises one or more amino residues e.g. 1 to 10, e.g. 1, 2, 3, 4, 5, 6, 7, 8, 9 or 10, of the following amino residues or consists of the following amino residues ENLNLIKAPM (SEQ ID NO. 303, amino acid No. 150-159 in Seq ID NO: 2).

In one embodiment, the epitope is determined by site directed mutagenesis as shown in the examples, for example using alanine scanning. In one embodiment, the epitope is a linear epitope. In one embodiment, the epitope is a conformational epitope. In one embodiment, the epitope includes a linear epitope in the large loop. In one embodiment, the epitope further includes a conformational epitope in the small loop. In one embodiment, the antibody is PMX003 or antigen binding portion thereof or an antibody with at least 80% sequence identity as described herein.

In another aspect of the invention, the invention also relates to an antibody or antigen binding portion thereof binds an epitope that comprises one or more amino residues, e.g. 1 to 19, e.g. 1, 2, 3, 4, 5, 6, 7, 8, 9, 10, 11, 12, 13, 14, 15, 16, 17, 18 or 19, of the following amino residues or consists of the following amino residues ITISHFFKMENLNLIKAPM (SEQ ID NO. 302, amino acid No. 141-159 in Seq ID NO. 2). In one embodiment, the epitope comprises one or more amino residues e.g. 1 to 10, e.g. 1, 2, 3, 4, 5, 6, 7, 8, 9 or 10, of the following amino residues or consists of the following amino residues ENLNLIKAPM (SEQ ID NO. 303, amino acid No. 150-159 in Seq ID NO. 2). For example, the antibody or antigen binding portion thereof may have the sequences as described herein.

The invention also relates to an antibody or antigen binding portion thereof that competes with an antibody or antigen binding portion thereof as described herein.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

The invention extends to antigen binding portions or antigen binding fragments of the antibody. The terms “binding portion” and “fragment” are used interchangeably herein. An antibody fragment is a portion of an antibody, for example a F(ab′)2, Fab, Fv, scFv, heavy chain, light chain, variable heavy (V_{H}), variable light (V_{L}) chain domain and the like. Functional fragments of a full-length antibody retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

The invention also extends to antibody mimetics that comprise a sequence as described herein.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments scFv fragments (˜25 kDa) that consist of the two variable domains, V_{H} and V_{L} connected into a single polypeptide chain. Naturally, V_{H} and V_{L} domains are non-covalently associated via hydrophobic interactions and tend to dissociate. However, stable fragments can be engineered by linking the domains with a hydrophilic flexible linker to create a single chain Fv (scFv).

The smallest antigen binding fragment is the single variable fragment, namely the variable heavy (V_{H}) or variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “immunoglobulin single variable domain”. A single domain antibody (˜12 to 15 kDa) has thus either the V_{H} or V_{L} domain, i.e. it does not have other parts of a full antibody. The term “dAb” for “domain antibodies” generally refers to a single immunoglobulin variable domain (V_{H}, V_{HH} or V_{L}) polypeptide that specifically binds antigen.

The term “isolated” refers to a moiety that is isolated from its natural environment. For example, the term “isolated” refers to an antibody or fragment thereof that is substantially free of other antibodies, antibodies or antibody fragments. Moreover, an isolated antibody may be substantially free of other cellular material and/or chemicals.

As used herein, the term “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percent identity between two amino acid sequences can be determined using well known mathematical algorithms.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

As used herein, a “substitution of an amino acid residue” with another amino acid residue in an amino acid sequence of heterodimeric protein or polypeptide as described herein (an antibody for example), is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence. By “wild type” or “WT” or “native” herein is meant an amino acid sequence or a nucleotide sequence that is found in nature, including allelic variations. A WT protein, polypeptide, antibody or immunoglobulin has an amino acid sequence or a nucleotide sequence that has not been intentionally modified.

The antibody or antigen-binding portion thereof according to the invention has one or more of the following properties:
binds specifically to canine CD20;
binds to canine CD20 with a KD as measured in the examples and for example as shown in the figures;
shows cell killing, such as CDC and/or ADCC, in canine lymphoma cell lines expressing CD20, as measured in the examples;
promotes antibody dependent cellular phagocytosis (ADCP);
is capable of effectively depleting CD20 positive B cells in canine tissues;
is capable of binding to cells expressing canine CD20;
is capable of depleting cells expressing canine CD20, suitably by direct cell killing via apoptosis;
provides good stability as shown in the examples;
binds an epitope that comprises one or more amino residues e.g. 1 to 10, e.g. 1, 2, 3, 4, 5, 6, 7, 8, 9 or 10, of the following amino residues or consists of the following amino residues ENLNLIKAPM (SEQ ID NO. 303, amino acid No. 150-159 in Seq ID NO. 2), for example ITISHFFKMENLNLIKAPM (SEQ ID NO. 302, amino acid No. 141-159 in Seq ID NO. 2);
has a stronger binding capacity (5-10 fold higher) to canine CD20 than benchmark test antibodies 1 E4 and 4E1-7, e.g. as shown in the examples;
has stronger ADCC and/or CDC activity than benchmark test antibodies 1 E4 and 4E1-7, e.g. as shown in the examples and in table 4 and/or
a more efficient killing of B cells in whole canine blood as compared to benchmark test antibodies 1 E4 and 4E1-7 mAbs, e.g. as shown in e.g. as shown in.

In one embodiment, the antibody or antigen-binding portion thereof according to the invention has one or more of the properties above and optionally one or more of the following properties:
a) has CDC activity with an EC50 value of less than 20 nM;
b) has ADCC activity with an EC50 value of less than 0.3 nM;
c) has a transient expression yield more than 100 μg/ml;
d) has a Tm1 as determined by Uncle higher than 58° C.;
e) provides in vivo cell killing in dog at a dose of about 0.5 mg/kg to 2.5 mg/kg;
f) maintains B cell depletion in vivo in dog at a low level for at least 15 days and/or
g) binds an epitope that comprises one or more amino residues e.g. 1 to 10, e.g. 1, 2, 3, 4, 5, 6, 7, 8, 9 or 10, of the following amino residues or consists of the following amino residues ENLNLIKAPM (SEQ ID NO. 303, amino acid No. 150-159 in Seq ID NO. 2), for example ITISHFFKMENLNLIKAPM (SEQ ID NO. 302, amino acid No. 141-159 in Seq ID NO. 2).

These properties can be measured by methods known in the art, such the methods disclosed in the examples, including in vivo studies in mouse models or in dogs.

In particular, the inventors have found that the antibodies and antigen binding portions thereof show both CDC and ADCC activity, e.g. as demonstrated by the cell killing in canine lymphoma cell lines expressing CD20, as measured in the examples.

In one aspect, the invention relates to an isolated canine antibody or antigen-binding portion thereof which binds canine CD20 wherein said antibody comprises
a) a heavy chain (HC) CDR1 sequence comprising or consisting of SEQ ID No. 27 or an amino acid sequence which has at least 60%, 70%, 80% or 90% sequence identity thereto,
b) a HC CDR2 sequence comprising or consisting of SEQ ID No. 28 or an amino acid sequence which has at least 60%, 70%, 80% or 90% sequence identity thereto,
c) a HC CDR3 sequence comprising or consisting of SEQ ID No. 29 or an amino acid sequence which has at least 60%, 70%, 80% or 90% sequence identity thereto,
d) a light chain (LC) CDR1 sequence comprising or consisting of SEQ ID No. 30 at least 60%, 70%, 80% or 90% sequence identity thereto,
e) a LC CDR2 sequence comprising or consisting of SEQ ID No. 31 or an amino acid sequence which has at least 60%, 70%, 80% or 90% sequence identity thereto and
f) a LC CDR3 sequence comprising or consisting of SEQ ID No. 32 or an amino acid sequence which has at least 60%, 70%, 80% or 90% sequence identity thereto.

The invention relates to an isolated canine antibody or antigen-binding portion thereof which binds canine CD20 wherein said antibody comprises
a) a HC CDR1 sequence comprising or consisting of SEQ ID No. 27
b) a HC CDR2 sequence comprising or consisting of SEQ ID No. 28
c) a HC CDR3 sequence comprising or consisting of SEQ ID No. 29
d) a LC CDR1 sequence comprising or consisting of SEQ ID No. 30,
e) a LC CDR2 sequence comprising or consisting of SEQ ID No. 31 and
f) a LC CDR3 sequence comprising or consisting of SEQ ID No. 32

or an isolated canine antibody or antigen-binding portion thereof with CDRs as described above but which has one or more CDR which has 1, 2, 3 or 4 amino acid substitutions compared to the CDR sequence mentioned above.

The invention thus relates to an isolated canine antibody or antigen-binding portion thereof which binds canine CD20 wherein said antibody comprises
a) a heavy chain (HC) CDR1 sequence comprising or consisting of SEQ ID No. 27 or an amino acid sequence which has 1 or 2 amino acid differences compared to SEQ ID No. 27,
b) a HC CDR2 sequence comprising or consisting of SEQ ID No. 28 or an amino acid sequence which has 1, 2, 3 or 4 amino acid differences compared to SEQ ID No. 28,
c) a HC CDR3 sequence comprising or consisting of SEQ ID No. 29 or an amino acid sequence which has 1 or 2 amino acid differences compared to SEQ ID No. 29,
d) a light chain (LC) CDR1 sequence comprising or consisting of SEQ ID No. 30 or an amino acid sequence which has 1 amino acid difference compared to SEQ ID No. 30,
e) a LC CDR2 sequence comprising or consisting of SEQ ID No. 31 or an amino acid sequence which has 1 amino acid difference compared to SEQ ID No. 31 and
f) a LC CDR3 sequence comprising or consisting of SEQ ID No. 32 or an amino acid sequence which has 1 or 2 amino acid differences compared to SEQ ID No. 32.

In one embodiment, the antibody or antigen-binding portion thereof has a HC CDR1 sequence comprising or consisting of SEQ ID No. 7, a HC CDR2 sequence comprising or consisting of SEQ ID No. 8, a HC CDR3 sequence comprising or consisting of SEQ ID No. 9, a LC CDR1 sequence comprising or consisting of SEQ ID No. 10, a LC CDR2 sequence comprising or consisting of SEQ ID No. 11 and a LC CDR3 sequence comprising or consisting of SEQ ID No. 12.

In one embodiment, the antibody or antigen-binding portion thereof has a HC CDR1 sequence comprising or consisting of SEQ ID No. 17, a HC CDR2 sequence comprising or consisting of SEQ ID No. 18, a HC CDR3 sequence comprising or consisting of SEQ ID No. 19, a LC CDR1 sequence comprising SEQ ID No. 20, a LC CDR2 sequence comprising or consisting of SEQ ID No. 21 and a LC CDR3 sequence comprising or consisting of SEQ ID No. 22.

In one embodiment, the antibody or antigen-binding portion thereof has a HC CDR1 sequence comprising SEQ ID No. 37, a HC CDR2 sequence comprising SEQ ID No. 38, a HC CDR3 sequence comprising SEQ ID No. 39, a LC CDR1 sequence comprising SEQ ID No. 40, a LC CDR2 sequence comprising SEQ ID No. 41 and a LC CDR3 sequence comprising SEQ ID No. 42.

In one embodiment, the antibody or antigen-binding portion thereof has a HC CDR1 sequence comprising or consisting of SEQ ID No. 47, a HC CDR2 sequence comprising or consisting of SEQ ID No. 48, a HC CDR3 sequence comprising or consisting of SEQ ID No. 49, a LC CDR1 sequence comprising or consisting of SEQ ID No. 50, a LC CDR2 sequence comprising or consisting of SEQ ID No. 51 and a LC CDR3 sequence comprising or consisting of SEQ ID No. 52.

In one embodiment, the antibody or antigen-binding portion thereof has a HC CDR1 sequence comprising or consisting of SEQ ID No. 82, a HC CDR2 sequence comprising or consisting of SEQ ID No. 83, a HC CDR3 sequence comprising or consisting of SEQ ID No. 84, a LC CDR1 sequence comprising or consisting of SEQ ID No. 85, a LC CDR2 sequence comprising or consisting of SEQ ID No. 86 and a LC CDR3 sequence comprising or consisting of SEQ ID No. 87.

In one embodiment, the antibody or antigen-binding portion thereof has a HC CDR1 sequence comprising or consisting of SEQ ID No. 92, a HC CDR2 sequence comprising or consisting of SEQ ID No. 93, a HC CDR3 sequence comprising or consisting of SEQ ID No. 94, a LC CDR1 sequence comprising or consisting of SEQ ID No. 95, a LC CDR2 sequence comprising or consisting of SEQ ID No. 96 and a LC CDR3 sequence comprising or consisting of SEQ ID No. 97.

In one embodiment, the antibody or antigen-binding portion thereof has a HC CDR1 sequence comprising or consisting of SEQ ID No. 102, a HC CDR2 sequence comprising or consisting of SEQ ID No. 103, a HC CDR3 sequence comprising or consisting of SEQ ID No. 104, a LC CDR1 sequence comprising or consisting of SEQ ID No. 105, a LC CDR2 sequence comprising or consisting of SEQ ID No. 106 and a LC CDR3 sequence comprising or consisting of SEQ ID No. 107.

In one embodiment, the antibody or antigen-binding portion thereof has a HC CDR1 sequence comprising or consisting of SEQ ID No. 112, a HC CDR2 sequence comprising or consisting of SEQ ID No. 113, a HC CDR3 sequence comprising or consisting of SEQ ID No. 114, a LC CDR1 sequence comprising or consisting of SEQ ID No. 115, a LC CDR2 sequence comprising or consisting of SEQ ID No. 116 and a LC CDR3 sequence comprising or consisting of SEQ ID No. 117.

In one embodiment, the antibody or antigen-binding portion thereof has a HC CDR1 sequence comprising or consisting of SEQ ID No. 122, a HC CDR2 sequence comprising or consisting of SEQ ID No. 123, a HC CDR3 sequence comprising or consisting of SEQ ID No. 124, a LC CDR1 sequence comprising or consisting of SEQ ID No. 125, a LC CDR2 sequence comprising or consisting of SEQ ID No. 126 and a LC CDR3 sequence comprising or consisting of SEQ ID No. 127.

In one embodiment, the antibody or antigen-binding portion thereof has a HC CDR1 sequence comprising or consisting of SEQ ID No. 132, a HC CDR2 sequence comprising or consisting of SEQ ID No. 133, a HC CDR3 sequence comprising or consisting of SEQ ID No. 134, a LC CDR1 sequence comprising or consisting of SEQ ID No. 135, a LC CDR2 sequence comprising or consisting of SEQ ID No. 136 and a LC CDR3 sequence comprising or consisting of SEQ ID No. 137.

In one embodiment, the antibody or antigen-binding portion thereof has a HC CDR1 sequence comprising or consisting of SEQ ID No. 142, a HC CDR2 sequence comprising or consisting of SEQ ID No. 143, a HC CDR3 sequence comprising or consisting of SEQ ID No. 144, a LC CDR1 sequence comprising or consisting of SEQ ID No. 145, a LC CDR2 sequence comprising or consisting of SEQ ID No. 146 and a LC CDR3 sequence comprising or consisting of SEQ ID No. 147.

In one embodiment, the antibody or antigen-binding portion thereof has a HC CDR1 sequence comprising or consisting of SEQ ID No. 152, a HC CDR2 sequence comprising or consisting of SEQ ID No. 153, a HC CDR3 sequence comprising or consisting of SEQ ID No. 154, a LC CDR1 sequence comprising or consisting of SEQ ID No. 155, a LC CDR2 sequence comprising or consisting of SEQ ID No. 156 and a LC CDR3 sequence comprising or consisting of SEQ ID No. 157.

In one embodiment, the antibody or antigen-binding portion thereof has a HC CDR1 sequence comprising or consisting of SEQ ID No. 162, a HC CDR2 sequence comprising or consisting of SEQ ID No. 163, a HC CDR3 sequence comprising or consisting of SEQ ID No. 164, a LC CDR1 sequence comprising or consisting of SEQ ID No. 165, a LC CDR2 sequence comprising or consisting of SEQ ID No. 166 and a LC CDR3 sequence comprising or consisting of SEQ ID No. 167.

In one embodiment, the antibody or antigen-binding portion thereof has a HC CDR1 sequence comprising or consisting of SEQ ID No. 172, a HC CDR2 sequence comprising or consisting of SEQ ID No. 173, a HC CDR3 sequence comprising or consisting of SEQ ID No. 174, a LC CDR1 sequence comprising or consisting of SEQ ID No. 175, a LC CDR2 sequence comprising or consisting of SEQ ID No. 176 and a LC CDR3 sequence comprising or consisting of SEQ ID No. 177.

In one embodiment, the antibody or antigen-binding portion thereof has a HC CDR1 sequence comprising or consisting of SEQ ID No. 182, a HC CDR2 sequence comprising or consisting of SEQ ID No. 183, a HC CDR3 sequence comprising or consisting of SEQ ID No. 184, a LC CDR1 sequence comprising or consisting of SEQ ID No. 185, a LC CDR2 sequence comprising or consisting of SEQ ID No. 186 and a LC CDR3 sequence comprising or consisting of SEQ ID No. 187.

In one embodiment, the antibody or antigen-binding portion thereof has a HC CDR1 sequence comprising or consisting of SEQ ID No. 192, a HC CDR2 sequence comprising or consisting of SEQ ID No. 193, a HC CDR3 sequence comprising or consisting of SEQ ID No. 194, a LC CDR1 sequence comprising or consisting of SEQ ID No. 195, a LC CDR2 sequence comprising or consisting of SEQ ID No. 196 and a LC CDR3 sequence comprising or consisting of SEQ ID No. 197.

In one embodiment, the antibody or antigen-binding portion thereof a HC CDR1 sequence comprising or consisting of SEQ ID No. 202, a HC CDR2 sequence comprising or consisting of SEQ ID No. 203, a HC CDR3 sequence comprising or consisting of SEQ ID No. 204, a LC CDR1 sequence comprising or consisting of SEQ ID No. 205, a LC CDR2 sequence comprising or consisting of SEQ ID No. 206 and a LC CDR3 sequence comprising or consisting of SEQ ID No. 207.

In one embodiment, the antibody or antigen-binding portion thereof has a HC CDR1 sequence comprising or consisting of SEQ ID No. 212, a HC CDR2 sequence comprising or consisting of SEQ ID No. 213, a HC CDR3 sequence comprising or consisting of SEQ ID No. 214, a LC CDR1 sequence comprising or consisting of SEQ ID No. 215, a LC CDR2 sequence comprising or consisting of SEQ ID No. 216 and a LC CDR3 sequence comprising or consisting of SEQ ID No. 217.

In one embodiment, the antibody or antigen-binding portion thereof has a HC CDR1 sequence comprising or consisting of SEQ ID No. 222, a HC CDR2 sequence comprising or consisting of SEQ ID No. 223, a HC CDR3 sequence comprising or consisting of SEQ ID No. 224, a LC CDR1 sequence comprising or consisting of SEQ ID No. 225, a LC CDR2 sequence comprising or consisting of SEQ ID No. 226 and a LC CDR3 sequence comprising or consisting of SEQ ID No. 227.

In one embodiment, the antibody or antigen-binding portion thereof has a HC CDR1 sequence comprising or consisting of SEQ ID No. 232, a HC CDR2 sequence comprising or consisting of SEQ ID No. 233, a HC CDR3 sequence comprising or consisting of SEQ ID No. 234, a LC CDR1 sequence comprising or consisting of SEQ ID No. 235, a LC CDR2 sequence comprising or consisting of SEQ ID No. 236 and a LC CDR3 sequence comprising or consisting of SEQ ID No. 237.

In one embodiment, the antibody or antigen-binding portion thereof comprises a heavy chain (HC) variable region sequence comprising SEQ ID NO. 24 or a sequence with at least 75%, 80%, 85% or 90% sequence identity thereto and a light chain (LC) variable region sequence comprising SEQ ID NO. 26 or a sequence with at least 70%, 75%, 80%, 85%, 90% or 95% sequence identity thereto. In one embodiment, the antibody or antigen-binding portion thereof comprises a heavy chain (HC) variable region sequence comprising SEQ ID NO. 24 and a light chain (LC) variable region sequence comprising SEQ ID NO. 26 or a LC variable region sequence comprising SEQ ID NO. 26 where the NVT sequence in FR1 of the LC variable region has been modified, for example an LC variable region with 1, 2 or 3 amino acid modifications in FR1, e.g. a LC variable region sequence comprising FR1 having SEQ ID NO. 60, 61, 62, 63, 64 or 65 as described below or a LC variable region sequence comprising SEQ ID NO. 53, 54, 55, 56, 57 or 58.

For example, the antibody or antigen-binding portion thereof has
a) a HC variable region sequence comprising or consisting of SEQ ID NO. 4 and a LC variable region sequence comprising or consisting of SEQ ID NO. 6;
b) a HC variable region sequence comprising or consisting of SEQ ID NO. 14 and a LC variable region sequence comprising or consisting of SEQ ID NO. 16;
c) a HC variable region sequence comprising SEQ ID NO. 34 and a LC variable region sequence comprising or consisting of SEQ ID NO. 36;
d) a HC variable region sequence comprising or consisting of SEQ ID NO. 44 and a LC variable region sequence comprising or consisting of SEQ ID NO. 46;
e) a HC variable region sequence comprising SEQ ID NO. 79 and a LC variable region sequence comprising SEQ ID NO. 81 or a LC variable region sequence where the NVT sequence as described below has been modified, e.g. a LC variable region sequence comprising SEQ ID NO. 238;
f) a HC variable region sequence comprising SEQ ID NO. 89 and a LC variable region sequence comprising SEQ ID NO. 91 or a LC variable region sequence where the NVT sequence as described below has been modified, e.g. a LC variable region sequence comprising SEQ ID NO. 239;
g) a HC variable region sequence comprising SEQ ID NO. 99 and a LC variable region sequence comprising SEQ ID NO. 101 or a LC variable region sequence where the NVT sequence as described below has been modified, e.g. a LC variable region sequence comprising SEQ ID NO. 240;
h) a HC variable region sequence comprising SEQ ID NO. 109 and a LC variable region sequence comprising SEQ ID NO. 111 or a LC variable region sequence where the NVT sequence as described below has been modified, e.g. a LC variable region sequence comprising SEQ ID NO. 241;
i) a HC variable region sequence comprising SEQ ID NO. 119 and a LC variable region sequence comprising SEQ ID NO. 121;
j) a HC variable region sequence comprising SEQ ID NO. 129 and a LC variable region sequence comprising SEQ ID NO. 131 or a LC variable region sequence where the NVT sequence as described below has been modified, e.g. a LC variable region sequence comprising SEQ ID NO. 242;
k) a HC variable region sequence comprising SEQ ID NO. 139 and a LC variable region sequence comprising SEQ ID NO. 141 or a LC variable region sequence where the NVT sequence as described below has been modified, e.g. a LC variable region sequence comprising SEQ ID NO. 243;
l) a HC variable region sequence comprising SEQ ID NO. 149 and a LC variable region sequence comprising SEQ ID NO. 151 or a LC variable region sequence where the NVT sequence as described below has been modified, e.g. a LC variable region sequence comprising SEQ ID NO. 244;
m) a HC variable region sequence comprising SEQ ID NO. 159 and a LC variable region sequence comprising SEQ ID NO. 161 or a LC variable region sequence where the NVT sequence as described below has been modified, e.g. a LC variable region sequence comprising SEQ ID NO. 245;
n) a HC variable region sequence comprising SEQ ID NO. 169 and a LC variable region sequence comprising SEQ ID NO. 171;
o) a HC variable region sequence comprising SEQ ID NO. 179 and a LC variable region sequence comprising SEQ ID NO. 181 or a LC variable region sequence where the NVT sequence as described below has been modified, e.g. a LC variable region sequence comprising SEQ ID NO. 246;
p) a HC variable region sequence comprising SEQ ID NO. 189 and a LC variable region sequence comprising SEQ ID NO. 191 or a LC variable region sequence where the NVT sequence as described below has been modified, e.g. a LC variable region sequence comprising SEQ ID NO. 247;
q) a HC variable region sequence comprising SEQ ID NO. 199 and a LC variable region sequence comprising SEQ ID NO. 201 or a LC variable region sequence where the NVT sequence as described below has been modified, e.g. a LC variable region sequence comprising SEQ ID NO. 248;
r) a HC variable region sequence comprising SEQ ID NO. 209 and a LC variable region sequence comprising SEQ ID NO. 211 or a LC variable region sequence where the NVT sequence as described below has been modified, e.g. a LC variable region sequence comprising SEQ ID NO. 249;
s) a HC variable region sequence comprising SEQ ID NO. 219 and a LC variable region sequence comprising SEQ ID NO. 221; or
t) a HC variable region sequence comprising SEQ ID NO. 229 and a LC variable region sequence comprising SEQ ID NO. 231 or a LC variable region sequence where the NVT sequence as described below has been modified, e.g. a LC variable region sequence comprising SEQ ID NO. 250.

In one embodiment, the antigen-binding portion thereof is a F(ab′)2, Fab, Fv, scFv, heavy chain, light chain, variable heavy (V_{H}) domain or variable light (V_{L}).

In another embodiment, the antibody or antigen-binding portion comprises a HC CDR1 sequence comprising or consisting of SEQ ID No. 27, a HC CDR2 sequence comprising or consisting of SEQ ID No. 28, a HC CDR3 sequence comprising or consisting of SEQ ID No. 29, a LCDR1 sequence comprising or consisting of SEQ ID No. 30, LC CDR2 sequence comprising or consisting of SEQ ID No. 31 and a LC CDR3 sequence comprising or consisting of SEQ ID No. 32. The antibody or antigen-binding portion has framework regions which have 5 amino acid changes in the heavy chain variable sequence as compared to germline sequence (SEQ ID NO: 74) and 1 amino acid change in the light chain variable sequence (SEQ ID NO: 76).

In one embodiment, the antibody or antigen-binding portion thereof has a HC CDR1 sequence comprising or consisting of SEQ ID No. 7, a HC CDR2 sequence comprising or consisting of SEQ ID No. 8, a HC CDR3 sequence comprising or consisting of SEQ ID No. 9, a LC CDR1 sequence comprising or consisting of SEQ ID No. 10, a LC CDR2 sequence comprising or consisting of SEQ ID No. 11 and a LC CDR3 sequence comprising or consisting of SEQ ID No. 12. The antibody or antigen-binding portion has framework regions which have 2 amino acid changes in the heavy chain variable sequence as compared to germline sequence (SEQ ID NO: 74) and 3 amino acid changes in light chain variable sequence as compared to the germline sequence (SEQ ID NO: 76).

In one embodiment, the antibody or antigen-binding portion thereof has a HC CDR1 sequence comprising or consisting of SEQ ID No. 17, a HC CDR2 sequence comprising or consisting of SEQ ID No. 18, a HC CDR3 sequence comprising or consisting of SEQ ID No. 19, a LC CDR1 sequence comprising SEQ ID No. 20, a LC CDR2 sequence comprising or consisting of SEQ ID No. 21 and a LC CDR3 sequence comprising or consisting of SEQ ID No. 22. The antibody or antigen-binding portion has framework regions which have 10 amino acid changes in the heavy chain variable sequence as compared to germline sequence (SEQ ID NO: 74) and no amino acid change in light chain variable sequence (SEQ ID NO: 76).

In one embodiment, the antibody or antigen-binding portion thereof has a HC CDR1 sequence comprising SEQ ID No. 37, a HC CDR2 sequence comprising SEQ ID No. 38, a HC CDR3 sequence comprising SEQ ID No. 39, a LC CDR1 sequence comprising SEQ ID No. 40, a LC CDR2 sequence comprising SEQ ID No. 41 and a LC CDR3 sequence comprising SEQ ID No. 42. The antibody or antigen-binding portion has framework regions which have 12 amino acid changes in the heavy chain variable sequence as compared to germline sequence (SEQ ID NO: 74) and no amino acid change in light chain variable sequence (SEQ ID NO: 76).

In one embodiment, the antibody or antigen-binding portion thereof has a HC CDR1 sequence comprising or consisting of SEQ ID No. 47, a HC CDR2 sequence comprising or consisting of SEQ ID No. 48, a HC CDR3 sequence comprising or consisting of SEQ ID No. 49, a LC CDR1 sequence comprising or consisting of SEQ ID No. 50, a LC CDR2 sequence comprising or consisting of SEQ ID No. 51 and a LC CDR3 sequence comprising or consisting of SEQ ID No. 52. The antibody or antigen-binding portion has framework regions which have 4 amino acid changes in the heavy chain variable sequence as compared to germline sequence (SEQ ID NO: 74) and no amino acid change in the light chain variable sequence (SEQ ID NO: 76).

In an embodiment the antibody or antigen-binding portion has framework regions which have 1 to 10 amino acid changes in the heavy chain variable sequence as compared to germline sequence (SEQ ID NO: 74). For example, the antibody or antigen-binding portion has framework regions may have 1, 2, 3, 4, 5, 6, 7, 8, 9 or 10 amino acid changes in the heavy chain variable sequence as compared to germline sequence (SEQ ID NO: 74). In an embodiment the antibody or antigen-binding portion has framework regions which have 1 to 10 amino acid changes in the light chain variable sequence as compared to germline sequence (SEQ ID NO: 76). For example, the antibody or antigen-binding portion has framework regions may have 1, 2, 3, 4, 5, 6, 7, 8, 9 or 10 amino acid changes in the light chain variable sequence as compared to germline sequence (SEQ ID NO: 76).

The invention also relates to an isolated canine antibody or antigen-binding portion thereof which binds canine CD20 wherein said antibody comprises
a) a HC CDR1 sequence comprising or consisting of SEQ ID No. 112 or an amino acid sequence which has 1, 2 or 3 amino acid differences compared to SEQ ID No. 112,
b) a HC CDR2 sequence comprising or consisting of SEQ ID No. 113 or an amino acid sequence which has 1, 2, 3, 4 or 5 amino acid differences compared to SEQ ID No. 113,
c) a HC CDR3 sequence comprising or consisting of SEQ ID No. 114 or an amino acid sequence which has 1, 2 or 3 amino acid differences compared to SEQ ID No. 114,
d) a LC CDR1 sequence comprising or consisting of SEQ ID No. 115 or an amino acid sequence which has 1 amino acid difference compared to SEQ ID No. 124,
e) a LC CDR2 sequence comprising or consisting of SEQ ID No. 116 or an amino acid sequence which has 1 or 2 amino acid differences compared to SEQ ID No. 116 and
f) a LC CDR3 sequence comprising or consisting of SEQ ID No. 117 or an amino acid sequence which has 1 or 2 amino acid differences compared to SEQ ID No. 117.

The invention also relates to an isolated canine antibody or antigen-binding portion thereof which binds canine CD20 wherein said antibody comprises
a) a HC CDR1 sequence comprising or consisting of SEQ ID No. 122 or an amino acid sequence which has 1, 2 or 3 amino acid differences compared to SEQ ID No. 122,
b) a HC CDR2 sequence comprising or consisting of SEQ ID No. 123 or an amino acid sequence which has 1, 2, 3 or 4 amino acid differences compared to SEQ ID No. 123,
c) a HC CDR3 sequence comprising or consisting of SEQ ID No. 124 or an amino acid sequence which has 1, 2, 3 or 4 amino acid differences compared to SEQ ID No. 124,
d) a LC CDR1 sequence comprising or consisting of SEQ ID No. 125 or an amino acid sequence which has 1 amino acid difference compared to SEQ ID No. 125,
e) a LC CDR2 sequence comprising or consisting of SEQ ID No. 126 or an amino acid sequence which has 1 amino acid difference compared to SEQ ID No. 126 and
f) a LC CDR3 sequence comprising or consisting of SEQ ID No. 127 or an amino acid sequence which has 1, 2 or 3 amino acid differences compared to SEQ ID No. 127.

Amino acid changes as used herein are selected from amino acid substitutions, additions or deletions. In one embodiment, an amino acid change is an amino acid substitution.

In one embodiment, the antibody is PMX001, PMX002, PMX003, PMX004, PMX005, PMX 006, PMX 007, PMX 008, PMX 009, PMX 010, PMX 011, PMX066, PMX067, PMX068, PMX069, PMX070, PMX071, PMX072, PMX073, PMX074, PMX075, PMX076, PMX077, PMX078, PMX079, PMX080, PMX081, PMX112, PMX113, PMX114, PMX115, PMX116, PMX117, PMX118, PMX119, PMX120, PMX121, PMX122, PMX123 or PMX 124 as shown in the examples and sequence information. In one embodiment, the antibody is selected from PMX001, PMX002, PMX003, PMX004, PMX005, PMX066, PMX067, PMX068, PMX069, PMX070, PMX071, PMX072, PMX073, PMX074, PMX075, PMX076, PMX077, PMX078, PMX079, PMX080 or PMX081. In one embodiment, the antibody is selected from PMX003, PMX066, PMX069, PMX070, PMX078, or PMX081. In one embodiment, the antibody is selected from PMX010, PMX112, PMX115, PMX122 or PMX124. In one embodiment, the antibody is selected from PMX069, PMX070 or PMX115.

Thus, in one embodiment, the antibody has a heavy chain variable region nucleotide sequence (SEQ ID NO: 3) that encodes for the corresponding amino acid sequence (SEQ ID NO: 4) and a light chain variable region nucleotide sequence (SEQ ID NO: 5) that encodes for the corresponding amino acid sequence (SEQ ID NO: 6). There are 2 amino acid changes in the heavy chain variable sequence as compared to germline sequence (SEQ ID NO: 74) and 3 amino acid changes in light chain variable sequence as compared to the germline sequence (SEQ ID NO: 76). The CDR sequences are as follows: CDR1 (SEQ ID NO: 7), CDR2 (SEQ ID NO: 8) and CDR3 (SEQ ID NO: 9) of the heavy chain variable region and the CDR1 (SEQ ID NO: 10), CDR2 (SEQ ID NO: 11) and CDR3 (SEQ ID NO: 12) of the light chain variable region.

In one embodiment, the antibody has a heavy chain variable region nucleotide sequence (SEQ ID NO: 13) that encodes for the corresponding amino acid sequence (SEQ ID NO: 14) and a light chain variable region nucleotide sequence (SEQ ID NO: 15) that encodes for the corresponding amino acid sequence (SEQ ID NO: 16). There are 10 amino acid changes in the heavy chain variable sequence as compared to germline sequence (SEQ ID NO: 74) and no amino acid change in light chain variable sequence (SEQ ID NO: 76). The CDRs are: CDR1 (SEQ ID NO: 17), CDR2 (SEQ ID NO: 18) and CDR3 (SEQ ID NO: 19) of the heavy chain variable region and the CDR1 (SEQ ID NO: 20), CDR2 (SEQ ID NO: 21) and CDR3 (SEQ ID NO: 22) of the light chain variable region.

In one embodiment, the antibody has a heavy chain variable region nucleotide sequence (SEQ ID NO: 23) that encodes for the corresponding amino acid sequence (SEQ ID NO: 24) and a light chain variable region nucleotide sequence (SEQ ID NO: 25) that encodes for the corresponding amino acid sequence (SEQ ID NO: 26). There are 5 amino acid changes in the heavy chain variable sequence as compared to germline sequence (SEQ ID NO: 74) and 1 amino acid change in light chain variable sequence (SEQ ID NO: 76). The CDRs are: CDR1 (SEQ ID NO: 27), CDR2 (SEQ ID NO: 28) and CDR3 (SEQ ID NO: 29) of the heavy chain variable region and the CDR1 (SEQ ID NO: 30), CDR2 (SEQ ID NO: 31) and CDR3 (SEQ ID NO: 32) of the light chain variable region.

In one embodiment, the antibody has a heavy chain variable region nucleotide sequence (SEQ ID NO: 33) that encodes for the corresponding amino acid sequence (SEQ ID NO: 34) and a light chain variable region nucleotide sequence (SEQ ID NO: 35) that encodes for the corresponding amino acid sequence (SEQ ID NO: 36). There are 12 amino acid changes in the heavy chain variable sequence as compared to germline sequence (SEQ ID NO: 74) and no amino acid change in light chain variable sequence (SEQ ID NO: 76). The CDRs are CDR1 (SEQ ID NO: 37), CDR2 (SEQ ID NO: 38) and CDR3 (SEQ ID NO: 39) of the heavy chain variable region and the CDR1 (SEQ ID NO: 40), CDR2 (SEQ ID NO: 41) and CDR3 (SEQ ID NO: 42) of the light chain variable region.

In one embodiment, the antibody has a heavy chain variable region nucleotide sequence (SEQ ID NO: 43) that encodes for the corresponding amino acid sequence (SEQ ID NO: 44) and a light chain variable region nucleotide sequence (SEQ ID NO: 45) that encodes for the corresponding amino acid sequence (SEQ ID NO: 46). There are 4 amino acid changes in the heavy chain variable sequence as compared to germline sequence (SEQ ID NO: 74) and no amino acid change in light chain variable sequence (SEQ ID NO: 76). The CDRs are CDR1 (SEQ ID NO: 47), CDR2 (SEQ ID NO: 48) and CDR3 (SEQ ID NO: 49) of the heavy chain variable region and the CDR1 (SEQ ID NO: 50), CDR2 (SEQ ID NO: 51) and CDR3 (SEQ ID NO: 52) of the light chain variable region.

In one embodiment, the antibody has a heavy chain variable region nucleotide sequence (SEQ ID NO: 78) that encodes for the corresponding amino acid sequence (SEQ ID NO: 79) and a light chain variable region nucleotide sequence (SEQ ID NO: 80) that encodes for the corresponding amino acid sequence (SEQ ID NO: 81). The CDRs are CDR1 (SEQ ID NO: 82), CDR2 (SEQ ID NO: 83) and CDR3 (SEQ ID NO: 84) of the heavy chain variable region and the CDR1 (SEQ ID NO: 85), CDR2 (SEQ ID NO: 86) and CDR3 (SEQ ID NO: 87) of the light chain variable region.

In one embodiment, the antibody has a heavy chain variable region nucleotide sequence (SEQ ID NO: 88) that encodes for the corresponding amino acid sequence (SEQ ID NO: 89) and a light chain variable region nucleotide sequence (SEQ ID NO: 90) that encodes for the corresponding amino acid sequence (SEQ ID NO: 91). The CDRs are CDR1 (SEQ ID NO: 92), CDR2 (SEQ ID NO: 93) and CDR3 (SEQ ID NO: 94) of the heavy chain variable region and the CDR1 (SEQ ID NO: 95), CDR2 (SEQ ID NO: 96) and CDR3 (SEQ ID NO: 97) of the light chain variable region.

In one embodiment, the antibody has a heavy chain variable region nucleotide sequence (SEQ ID NO: 98) that encodes for the corresponding amino acid sequence (SEQ ID NO: 99) and a light chain variable region nucleotide sequence (SEQ ID NO: 100) that encodes for the corresponding amino acid sequence (SEQ ID NO: 101). The CDRs are CDR1 (SEQ ID NO: 102), CDR2 (SEQ ID NO: 103) and CDR3 (SEQ ID NO: 104) of the heavy chain variable region and the CDR1 (SEQ ID NO: 105), CDR2 (SEQ ID NO: 106) and CDR3 (SEQ ID NO: 107) of the light chain variable region.

In one embodiment, the antibody has a heavy chain variable region nucleotide sequence (SEQ ID NO: 108) that encodes for the corresponding amino acid sequence (SEQ ID NO: 109) and a light chain variable region nucleotide sequence (SEQ ID NO: 110) that encodes for the corresponding amino acid sequence (SEQ ID NO: 111). The CDRs are CDR1 (SEQ ID NO: 112), CDR2 (SEQ ID NO: 113) and CDR3 (SEQ ID NO: 114) of the heavy chain variable region and the CDR1 (SEQ ID NO: 115), CDR2 (SEQ ID NO: 116) and CDR3 (SEQ ID NO: 117) of the light chain variable region.

In one embodiment, the antibody has a heavy chain variable region nucleotide sequence (SEQ ID NO: 118) that encodes for the corresponding amino acid sequence (SEQ ID NO: 119) and a light chain variable region nucleotide sequence (SEQ ID NO: 120) that encodes for the corresponding amino acid sequence (SEQ ID NO: 121). The CDRs are CDR1 (SEQ ID NO: 122), CDR2 (SEQ ID NO: 123) and CDR3 (SEQ ID NO: 124) of the heavy chain variable region and the CDR1 (SEQ ID NO: 125), CDR2 (SEQ ID NO: 126) and CDR3 (SEQ ID NO: 127) of the light chain variable region.

In one embodiment, the antibody has a heavy chain variable region nucleotide sequence (SEQ ID NO: 128) that encodes for the corresponding amino acid sequence (SEQ ID NO: 129) and a light chain variable region nucleotide sequence (SEQ ID NO: 130) that encodes for the corresponding amino acid sequence (SEQ ID NO: 131). The CDRs are CDR1 (SEQ ID NO: 132), CDR2 (SEQ ID NO: 133) and CDR3 (SEQ ID NO: 134) of the heavy chain variable region and the CDR1 (SEQ ID NO: 135), CDR2 (SEQ ID NO: 136) and CDR3 (SEQ ID NO: 137) of the light chain variable region.

In one embodiment, the antibody has a heavy chain variable region nucleotide sequence (SEQ ID NO: 138) that encodes for the corresponding amino acid sequence (SEQ ID NO: 139) and a light chain variable region nucleotide sequence (SEQ ID NO: 140) that encodes for the corresponding amino acid sequence (SEQ ID NO: 141). The CDRs are CDR1 (SEQ ID NO: 142), CDR2 (SEQ ID NO: 143) and CDR3 (SEQ ID NO: 144) of the heavy chain variable region and the CDR1 (SEQ ID NO: 145), CDR2 (SEQ ID NO: 146) and CDR3 (SEQ ID NO: 147) of the light chain variable region.

In one embodiment, the antibody has a heavy chain variable region nucleotide sequence (SEQ ID NO: 148) that encodes for the corresponding amino acid sequence (SEQ ID NO: 149) and a light chain variable region nucleotide sequence (SEQ ID NO: 150) that encodes for the corresponding amino acid sequence (SEQ ID NO: 151). The CDRs are CDR1 (SEQ ID NO: 152), CDR2 (SEQ ID NO: 153) and CDR3 (SEQ ID NO: 154) of the heavy chain variable region and the CDR1 (SEQ ID NO: 155), CDR2 (SEQ ID NO: 156) and CDR3 (SEQ ID NO: 157) of the light chain variable region.

In one embodiment, the antibody has a heavy chain variable region nucleotide sequence (SEQ ID NO: 158) that encodes for the corresponding amino acid sequence (SEQ ID NO: 159) and a light chain variable region nucleotide sequence (SEQ ID NO: 160) that encodes for the corresponding amino acid sequence (SEQ ID NO: 161). The CDRs are CDR1 (SEQ ID NO: 162), CDR2 (SEQ ID NO: 163) and CDR3 (SEQ ID NO: 164) of the heavy chain variable region and the CDR1 (SEQ ID NO: 165), CDR2 (SEQ ID NO: 166) and CDR3 (SEQ ID NO: 167) of the light chain variable region.

In one embodiment, the antibody has a heavy chain variable region nucleotide sequence (SEQ ID NO: 168) that encodes for the corresponding amino acid sequence (SEQ ID NO: 169) and a light chain variable region nucleotide sequence (SEQ ID NO: 170) that encodes for the corresponding amino acid sequence (SEQ ID NO: 171). The CDRs are CDR1 (SEQ ID NO: 172), CDR2 (SEQ ID NO: 173) and CDR3 (SEQ ID NO: 174) of the heavy chain variable region and the CDR1 (SEQ ID NO: 175), CDR2 (SEQ ID NO: 176) and CDR3 (SEQ ID NO: 177) of the light chain variable region.

In one embodiment, the antibody has a heavy chain variable region nucleotide sequence (SEQ ID NO: 178) that encodes for the corresponding amino acid sequence (SEQ ID NO: 179) and a light chain variable region nucleotide sequence (SEQ ID NO: 180) that encodes for the corresponding amino acid sequence (SEQ ID NO: 181). The CDRs are CDR1 (SEQ ID NO: 182), CDR2 (SEQ ID NO: 183) and CDR3 (SEQ ID NO: 184) of the heavy chain variable region and the CDR1 (SEQ ID NO: 185), CDR2 (SEQ ID NO: 186) and CDR3 (SEQ ID NO: 187) of the light chain variable region.

In one embodiment, the antibody has a heavy chain variable region nucleotide sequence (SEQ ID NO: 188) that encodes for the corresponding amino acid sequence (SEQ ID NO: 189) and a light chain variable region nucleotide sequence (SEQ ID NO: 190) that encodes for the corresponding amino acid sequence (SEQ ID NO: 191). The CDRs are CDR1 (SEQ ID NO: 192), CDR2 (SEQ ID NO: 193) and CDR3 (SEQ ID NO: 194) of the heavy chain variable region and the CDR1 (SEQ ID NO: 195), CDR2 (SEQ ID NO: 196) and CDR3 (SEQ ID NO: 197) of the light chain variable region.

In one embodiment, the antibody has a heavy chain variable region nucleotide sequence (SEQ ID NO: 198) that encodes for the corresponding amino acid sequence (SEQ ID NO: 199) and a light chain variable region nucleotide sequence (SEQ ID NO: 200) that encodes for the corresponding amino acid sequence (SEQ ID NO: 201). The CDRs are CDR1 (SEQ ID NO: 202), CDR2 (SEQ ID NO: 203) and CDR3 (SEQ ID NO: 204) of the heavy chain variable region and the CDR1 (SEQ ID NO: 205), CDR2 (SEQ ID NO: 206) and CDR3 (SEQ ID NO: 207) of the light chain variable region.

In one embodiment, the antibody has a heavy chain variable region nucleotide sequence (SEQ ID NO: 208) that encodes for the corresponding amino acid sequence (SEQ ID NO: 209) and a light chain variable region nucleotide sequence (SEQ ID NO: 210) that encodes for the corresponding amino acid sequence (SEQ ID NO: 211). The CDRs are CDR1 (SEQ ID NO: 212), CDR2 (SEQ ID NO: 213) and CDR3 (SEQ ID NO: 214) of the heavy chain variable region and the CDR1 (SEQ ID NO: 215), CDR2 (SEQ ID NO: 216) and CDR3 (SEQ ID NO: 217) of the light chain variable region.

In one embodiment, the antibody has a heavy chain variable region nucleotide sequence (SEQ ID NO: 218) that encodes for the corresponding amino acid sequence (SEQ ID NO: 219) and a light chain variable region nucleotide sequence (SEQ ID NO: 220) that encodes for the corresponding amino acid sequence (SEQ ID NO: 221). The CDRs are CDR1 (SEQ ID NO: 222), CDR2 (SEQ ID NO: 223) and CDR3 (SEQ ID NO: 224) of the heavy chain variable region and the CDR1 (SEQ ID NO: 225), CDR2 (SEQ ID NO: 226) and CDR3 (SEQ ID NO: 227) of the light chain variable region.

In one embodiment, the antibody has a heavy chain variable region nucleotide sequence (SEQ ID NO: 228) that encodes for the corresponding amino acid sequence (SEQ ID NO: 229) and a light chain variable region nucleotide sequence (SEQ ID NO: 230) that encodes for the corresponding amino acid sequence (SEQ ID NO: 231). The CDRs are CDR1 (SEQ ID NO: 232), CDR2 (SEQ ID NO: 233) and CDR3 (SEQ ID NO: 234) of the heavy chain variable region and the CDR1 (SEQ ID NO: 235), CDR2 (SEQ ID NO: 236) and CDR3 (SEQ ID NO: 237) of the light chain variable region.

In one embodiment, the antibody or antigen-binding portion thereof comprises an Fc region, for example a canine Fc region, for example a canine IgGB Fc region.

The variable region sequences described herein, including but not limited to the amino acid and nucleotide sequences shown in Table 2 (and/or fragments thereof) may be used in combination with one or more amino acid sequences and/or nucleotide sequences encoding one or more constant chains (and/or a fragment thereof) of an antibody molecule. For instance, the variable region amino acid sequences shown in Table 2 may be joined to the constant regions of any antibody molecule of the same or a different species (e.g., human, goat, rat, sheep, chicken) of that from which the variable region amino acid sequence was derived. Preferably, the variable region amino acid sequences shown in Table 2 is joined to the constant regions of a canine antibody and may be the constant region from any of canine IgG A, B, C or D. In one embodiment, the constant region is canine IgG B constant region. Dog IGGB (SEQ ID NO: 66), dog IGK (SEQ ID NO: 73) or dog IGLC5 (SEQ ID NO: 67) constant regions may be used.

Also within the scope of the invention are variants of the antibodies and antigen binding portions as described above.

A variant of an antibody or antigen binding portion thereof as described herein has at least 80%, 90%, 91%, 92%, 93%, 94%, 95%, 96%, 97%, 98% or 99% sequence identity to the non-variant molecule. In one embodiment, sequence identity is at least 95%. In one embodiment, the modification is a conservative sequence modification.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody or antigen binding portion thereof of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., CD20 binding) using the functional assays described herein.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues that comprise polypeptides and peptides described herein, and conservative substitutions for these amino acid residues are shown in Table 1 below.

TABLE 1
Amino Acid Residues and Examples of
Conservative Amino Acid Substitutions

| Original residue | |
|---|---|
| Three letter code, single letter code | Conservative substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidine, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

In some embodiments, the invention provides an antibody or antigen binding portion thereof that is a variant of an antibody or antigen binding portion thereof compared to a sequence described herein, e.g. selected from SEQ ID NO. 3 to 65 or SEQ ID NO. 78 to 237 that comprises one or more sequence modification and has improvements in one or more of a property such as binding affinity, specificity, thermostability, expression level, effector function, glycosylation, reduced immunogenicity, or solubility as compared to the unmodified antibody or fragment thereof.

Suitable methods for measuring properties which may suggest that the antibody can be successfully developed at scale include first purification using chromatography, such as affinity chromatography chromatography (Protein A: MabSelect Sure LX), anion exchange chromatography (Capto Q), cation exchange chromatography (Capto S) and buffer exchange (G-25 Fine), followed by assessment of whether the antibody remains intact (e.g. using SDS PAGE analysis to determine molecular weight, HPLC-SEC to calculate % of monomers, assess aggregation, and thermostability (Tm) studies.

For example, consensus sequence for asparagine-linked glycosylation of proteins can be removed. The framework region 1 (FR1) of the light chain of PMX003 (SEQ ID NO: 26) contains the NVT sequon, which is a consensus sequence for asparagine-linked glycosylation of proteins. To remove the glycans bound to this site, the NVT sequon in the FR1 of PMX003 mAb was mutated to QVT for PMX006 mAb, AVT for PMX007 mAb, EVT for PMX008 mAb, NVA for PMX009 mAb, SVT for PMX010 mAb and TVT for PMX011 mAb. Such modifications are within the scope of the invention.

The resulted light chain variable region amino acid sequences of PMX006 mAb (SEQ ID NO: 53), PMX007 mAb (SEQ ID NO: 54), PMX008 mAb (SEQ ID NO: 55), PMX009 mAb (SEQ ID NO: 56), PMX010 mAb (SEQ ID NO: 57), PMX011 mAb (SEQ ID NO: 58) and the FR1 sequences of PMX003 (SEQ ID NO: 59), PMX006 mAb (SEQ ID NO: 60), PMX007 mAb (SEQ ID NO: 61), PMX008 mAb (SEQ ID NO: 62), PMX009 mAb (SEQ ID NO: 63), PMX010 mAb (SEQ ID NO: 64), PMX011 mAb (SEQ ID NO: 65) are within the scope of the invention and are listed in the Sequences Table 2. These may be used with the PMX003 heavy chain instead of SEQ ID NO: 26. The LC FR1 sequence for PMX003 is shown in SEQ ID NO. 59, the modified LC FR1 sequences in SEQ ID Nos. 60, 61, 62, 63, 64 and 65.

The NVT sequon is found at residue position 11 to 13 of FR1 of the light chain of PMX001 to PMX005 (SEQ ID NO:6, SEQ ID NO:16, SEQ ID NO:26, SEQ ID NO:36, SEQ ID NO:46) and PMX066 to PMX0081 (SEQ ID NO:81, SEQ ID NO:91, SEQ ID NO:101, SEQ ID NO:111, SEQ ID NO:121, SEQ ID NO:131, SEQ ID NO:141, SEQ ID NO:151, SEQ ID NO:161, SEQ ID NO:171, SEQ ID NO:181, SEQ ID NO:191, SEQ ID NO:201, SEQ ID NO:211, SEQ ID NO:221, SEQ ID NO:231). The NVT sequon found at residue position 11 to 13 of FR1 of the light chain of PMX001 to PMX005 or PMX066 to PMX081 may be mutated to a sequence selected from: QVT, AVT, EVT, NVA, SVT, TVT. In an embodiment the NVT sequon found at residue position 11 to 13 of FR1 of the light chain of PMX001 to PMX005 or PMX066 to PMX081 is mutated to SVT.

The framework region 1 (FR1) of the light chain of PMX066 (SEQ ID NO: 81), PMX067 (SEQ ID NO: 91), PMX068 (SEQ ID NO: 101), PMX069 (SEQ ID NO: 111), PMX071 (SEQ ID NO: 131), PMX072 (SEQ ID NO: 141), PMX073 (SEQ ID NO: 151), PMX074 (SEQ ID NO: 161), PMX076 (SEQ ID NO: 181), PMX077 (SEQ ID NO: 191), PMX078 (SEQ ID NO: 201), PMX079 (SEQ ID NO: 211), PMX081 (SEQ ID NO: 231) contain the NVT sequon, which is a consensus sequence for asparagine-linked glycosylation of proteins. To remove the glycans bound to this site, the NVT sequons in the FR1 of PMX066, PMX067, PMX068, PMX069, PMX071, PMX072, PMX073, PMX074, PMX076, PMX077, PMX078, PMX079 and PMX081 mAbs were mutated to SVT to produce PMX112 (SEQ ID NO: 238), PMX113 (SEQ ID NO: 239), PMX114 (SEQ ID NO: 240), PMX115 (SEQ ID NO: 241), PMX116 (SEQ ID NO: 242), PMX117 (SEQ ID NO: 243), PMX118 (SEQ ID NO: 244), PMX119 (SEQ ID NO: 245), PMX120 (SEQ ID NO: 246), PMX121 (SEQ ID NO: 247), PMX122 (SEQ ID NO: 248), PMX123 (SEQ ID NO: 249) and PMX124 (SEQ ID NO: 250) respectively. Such modifications are within the scope of the invention the light chain of these can be used with their respective heavy chain counterpart, e.g. the HC of PMX066 (SEQ ID NO: 79) may be used with the modified LC (SEQ ID NO: 238) and so forth.

The resulting light chain variable region amino acid sequences of PMX112 mAb (SEQ ID NO: 238), PMX113 mAb (SEQ ID NO: 239), PMX114 mAb (SEQ ID NO: 240), PMX115 mAb (SEQ ID NO: 241), PMX116 mAb (SEQ ID NO: 242), PMX117 mAb (SEQ ID NO: 243), PMX118 mAb (SEQ ID NO: 244), PMX119 mAb (SEQ ID NO: 245), PMX120 mAb (SEQ ID NO: 246), PMX121 mAb (SEQ ID NO: 247), PMX122 mAb (SEQ ID NO: 248), PMX123 mAb (SEQ ID NO: 249) and PMX124 mAb (SEQ ID NO: 250) are within the scope of the invention and are listed in the Sequences Table.

A skilled person will know that there are different ways to identify, obtain and optimise the antigen binding molecules as described herein, including in vitro and in vivo expression libraries. This is further described in the examples. Optimisation techniques known in the art, such as display (e.g., ribosome and/or phage display) and/or mutagenesis (e.g., error-prone mutagenesis) can be used. The invention therefore also comprises sequence optimised variants of the antibodies described herein.

In one embodiment, modifications can be made to decrease the immunogenicity of the antibody. For example, one approach is to revert one or more framework residues to the corresponding canine germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. In one embodiment, all framework sequences are germline sequence.

To return one or more of the amino acid residues in the framework region sequences to their germline configuration, the somatic mutations can be “backmutated” to the germline sequence by, for example, site-directed mutagenesis or PCR-mediated mutagenesis.

Another type of framework modification involves mutating one or more residues within the framework region, or even within one or more CDR regions, to remove T cell epitopes to thereby reduce the potential immunogenicity of the antibody.

In some embodiments, the antigen-binding proteins, fragments and derivatives thereof, and fusion proteins of the present disclosure undergo post-translational modifications, for example but not limited to, a glutamine can be cyclized or converted to pyroglutamic acid; additionally, or alternatively, amino acids can undergo deamidation, isomerization, glycation and/or oxidation. The polypeptides of the present disclosure can undergo additional post-translational modification, including glycosylation, for example N-linked or O-linked glycosylation, at sites that are well-known in the art. Changes can be made in the amino acid sequence of a polypeptide to preclude or minimize such alterations, or to facilitate them in circumstances where such processing is beneficial. Polypeptides of the present disclosure include polypeptides that have been modified, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties.

Thus, in still another embodiment, glycosylation is modified. For example, an aglycoslated antibody can be made (i.e., the antibody lacks glycosylation). In one embodiment, the light chain variable region amino acid sequences of the aglycosylated antibody comprises SEQ ID NO: 53, SEQ ID NO: 54, SEQ ID NO: 55, SEQ ID NO: 56, SEQ ID NO: 57, SEQ ID NO: 58. SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250. FR1 sequences are provided as SEQ ID NO: 59, SEQ ID NO: 60, SEQ ID NO: 61, SEQ ID NO: 62, SEQ ID NO: 63, SEQ ID NO: 64, or SEQ ID NO: 65.

Glycosylation can also be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for the antigen.

In some applications, the binding agents may bind canine CD20 but have altered ability to bind Fc receptors as compared to standard binding agents. In one example, the binding agents are antibodies that have modified glycosylation patterns. IgG molecules, for example, typically contain N-linked oligosaccharides, for example fucose.

In one embodiment, the antibody or antigen-binding portion thereof is a-fucosylated. In cancer immunotherapy, antibodies may rely on the Fc-mediated immune effector function, antibody-dependent cellular cytotoxicity (ADCC), as the major mode of action to deplete tumor cells. It is well-known that this effector function is modulated by the N-linked glycosylation in the Fc region of the antibody. In particular, absence of core fucose on the Fc N-glycan has been shown to increase IgG1 Fc binding affinity to the FcγRIIIa present on immune effector cells such as natural killer cells and lead to enhanced ADCC activity. Therefore, a-fucosylated antibodies may have advantageous to improve therapeutic efficacy and absence/removal of the fucose enhances the ability of the antibody to interact with Fc receptors. Antibodies of this type may be referred to as “a-fucosylated”. Such antibodies may be produced using techniques described herein and/or that may be known in the art. In some embodiments, a nucleic acid sequence encoding an antibody may be expressed in a cell line that has modified glycosylation abilities (e.g., deleted, modified or lesser amount of fucosyl transferase) and fail to add the typical fucose moieties.

In one embodiment, the antibody or antigen-binding portion thereof has a CDC activity with an EC50 value in the 0.9-4.5 ug/ml (6-30 nM) range, e.g. less than 20 nM.

In one embodiment, the antibody or antigen-binding portion thereof has an ADCC activity with an EC50 value of less than 0.3 nM, e.g 0.013 ug/ml (0.09 nM).

In one embodiment, the antibody or antigen-binding portion thereof according to the invention has one or more of the following properties:
a) has CDC activity with an EC50 value of less than 20 nM;
b) has ADCC activity with an EC50 value of less than 0.3 nM;
c) has a transient expression yield more than 100 μg/ml;
d) has a Tm1 as determined by Uncle higher than 58° C. and/or
e) provides in vivo cell killing in dog at a dose of about 0.5 mg/kg to 2.5 mg/kg
f) maintains B cell depletion in vivo in dog at a low level for at least 15 days and/or
g) binds an epitope that comprises one or more amino residues e.g. 1 to 10, e.g. 1, 2, 3, 4, 5, 6, 7, 8, 9 or 10, of the following amino residues or consists of the following amino residues ENLNLIKAPM (SEQ ID NO. 303, amino acid No. 150-159 in Seq ID NO: 2), for example ITISHFFKMENLNLIKAPM (SEQ ID NO. 302 amino acid No. 141-159 in Seq ID NO: 2).

ADCC and CDC activity may be measured as described in the examples.

In one embodiment, the antibody and antigen binding portion thereof shows both CDC and ADCC activity, e.g. as demonstrated by the cell killing in canine lymphoma cell lines expressing CD20, as measured in the examples.

In one embodiment, the antibody or antigen-binding portion thereof has one or more of the properties as set out above and has the HC CDRs and LC CDRs and/or the HC and LC CDRS of PMX069, PMX115 or PMX070. In one embodiment, the antibody or antigen-binding portion thereof is selected from PMX069, PMX115 or PMX070.

In one embodiment, the antibody is the antibody described in Table 4 or an antigen-binding portion thereof and has an EC50 value for ADCC or CDC as shown in that table. EC50 values may be measured as described in the examples.

In one embodiment, the Fc portion of the antibody may be modified.

In one embodiment, the one or more substitution in the variant is in the CDR1, 2 and/or 3 region. For example, there may be 1, 2, 3, 4, 5 or more amino acid substitutions in the CDR1, 2 and/or 3 region. In another example, there may be 1 or 2 amino acid deletions.

In one embodiment, the one or more substitution is in the framework region. For example, there may be 1 to 20, e.g. 1 to 10, e.g. 1, 2, 3, 4, 5, 6, 7, 8, 9 or 10, amino acid substitutions in the HC variable region and/or LC variable region framework region.

The antibodies of the invention preferably have KD, IC50 and/or EC50 values, e.g. a KD as further described herein in the examples. Suitably, the KD value is sufficient for the antibodies to have the desired biological effect. For example, the KD can be least about 10 pM to 100 uM, about 100 pM to 10 nM or higher. The EC50 value may be as in Table 4, for example 0.12 to 0.89 nM. KD, IC50 and/or EC50 values may be measured as described in the examples.

The term “KD” refers to the “equilibrium dissociation constant” and refers to the value obtained in a titration measurement at equilibrium, or by dividing the dissociation rate constant (Koff) by the association rate constant (Kon). “KA” refers to the affinity constant. The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® SPR assay can be used. The invention also relates to an isolated canine antibody or antigen-binding portion thereof that binds to canine CD20 competing with an antibody or antigen-binding portion thereof as described above. Antibodies, antibody fragments or antibody mimetics that bind at or near the same epitope or an overlapping epitope on canine CD20 as any of the CD20 antibodies of the invention have the ability to cross-compete for binding to CD20 with any of the antibodies of the invention. The antibodies of the invention can thus be used as a reference antibody to assess such cross-reactivity. Such cross-competing antibodies can be identified based on their ability to cross-compete with an antibody described herein in standard CD20 binding assays. For example, BIAcore® analysis, ELISA assays or flow cytometry may be used to demonstrate cross-competition with the antibodies.

### Nucleic Acid Sequences, Vectors and Host Cells

The invention also relates to a nucleic acid sequence that encodes an amino acid sequence of an antibody or antigen binding portion thereof as described herein, e.g. a HC variable region or LC variable region. Exemplary sequences are described in table 2. In one embodiment, said nucleic acid is selected from SEQ ID NOs. 3, 5, 13, 15, 23, 25, 33, 35, 43, 45, 78, 80, 88, 90, 98, 100, 108, 110, 118, 120, 128, 130, 138, 140, 148, 150, 158, 160, 168, 170, 178, 180, 188, 190, 198, 200, 208, 210, 218, 220, 228, 230, 251, 252, 253, 254, 255, 256, 257, 258, 259, 260, 261, 262, 263, 264, 265, 266, 267, 268, 269, 270,271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or 301 or a nucleic acid having at least 75%, 80% or 90% sequence homology thereto. In one embodiment, said nucleic acid sequence is linked with a linker to a second nucleic acid sequence. In one embodiment, said second nucleic acid encodes an additional therapeutic moiety. In one embodiment, said linker is a nucleic acid linker. An exemplary nucleic acid is shown below. However, a skilled person will understand that due to the degeneracy of the genetic code, other sequences are envisaged.

Codon optimised nucleotide sequences are also within the scope of the invention, including SEQ ID NOs. 68 and 69.

A nucleic acid according to the present invention may comprise DNA or RNA and may be wholly or partially synthetic or recombinantly produced. Reference to a nucleotide sequence as set out herein encompasses a DNA molecule with the specified sequence, and encompasses a RNA molecule with the specified sequence in which U is substituted for T, unless context requires otherwise.

Furthermore, the invention relates to a nucleic acid construct comprising at least one nucleic acid as defined above. The construct may be in the form of a plasmid, vector, transcription or expression cassette.

The invention also relates to a vector that comprises a nucleic acid encoding the CD20 binding molecules as described herein. The term “vector” refers to a nucleic acid molecule, preferably a DNA molecule derived, for example, from a plasmid, bacteriophage, or vims, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art. In an embodiment the vector is an adeno-associated virus (AAV) vector, such as those described in WO2021176362.

In some embodiments, the nucleic acid may also comprise a leader sequence. In another embodiment, it does not comprise a leader sequence. Any suitable leader sequences may be used including the native immunoglobulin germline leader sequence, such as SEQ ID NO: 71 for heavy chain, SEQ ID NO: 72 for light chain of PMX001 to PMX005 and PMX066 to PMX081 mAbs, or others, such as the Campath leader sequence (SEQ ID NO: 70) (see U.S. Pat. No. 8,362,208 B2), may be chosen to enhance protein expression.

In some embodiments, the nucleic acid may also comprise a signal peptide, i.e. a short amino acid sequence (13-36 amino acids) on the N-terminus of a secretory protein (like an immunoglobulin) that mediates the translocation of a protein destined for secretion through the first membrane of the secretory pathway. This sequence is not present in the mature protein, being cleaved in a co-translational event, but mediates the secretion and correct expression of the protein. Suitable signal sequences can be used to optimize the expression of a recombinant protein.

The invention also relates to an isolated recombinant host cell comprising one or more nucleic acid construct as described above. Host cells useful in the present invention are prokaryotic, yeast, or higher eukaryotic cells and include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g. Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Prokaryotes useful as host cells in the present invention include gram negative or gram-positive organisms such as E. coli, B. subtilis, Enterobacter, Erwinia, Klebsiella, Proteus, Salmonella, Serratia, and Shigella, as well as Bacilli, Pseudomonas, and Streptomyces. One cloning host is E. coli 294 (ATCC 31,446), although other strains such as E. coli B, E. coli X1776 (ATCC 31,537), and E. coli W3110 (ATCC 27,325) are suitable. In one embodiment, a method of making an anti-CD20 antibody as described herein is provided, wherein the method comprises culturing the host cell under conditions suitable for expression of the polynucleotide encoding the antibody and isolating the antibody.

Nucleic acids encoding antibodies can be used to administer an antibody to an individual in order to produce their encoded protein in vivo and mediate a therapeutic effect. Delivery of polynucleotides into a subject can be direct such that polynucleotides or expression vectors are administered to an individual e.g. through introduction of mRNA or DNA directly into cells e.g. muscle cells. Indirect introduction is also envisaged where polynucleotides are transformed into cells in vitro prior to administration. Viral vectors, such as defective or attenuated viruses, may also be used.

In one embodiment, a method of making an anti-CD20 antibody as described herein is provided, wherein the method comprises culturing the host cell under conditions suitable for expression of the polynucleotide encoding the antibody and isolating the antibody.

The invention also relates to a heterologous assay or expression system comprising a canine CD20 and a cell line derived from a different species, e.g. a human cell line such as HEK.

The assay comprises contacting a canine CD20 with a cell line derived from a different species, e.g. a cell line from a different mammal, e.g. a rodent cell line or a human cell line such as HEK. For example, the cell line is transfected with canine CD20 such that it expresses canine CD20 in a stable or transient manner.

### Immunoconjugates and Other Binding Agents

The invention relates to immunoconjugates and other binding agents comprising the antibody or antigen binding portion thereof according to the invention. For example, the antibody or antigen-binding portion thereof according to the invention may be conjugated to a therapeutic moiety or non-therapeutic moiety.

In one embodiment, the therapeutic moiety is a binding molecule that binds to a target antigen of interest, for example selected from an antibody or antibody fragment (e.g., a Fab, F(ab′)2, Fv, a single chain Fv fragment (scFv) or single domain antibody, for example a V_{H} or V_{HH} domain) or antibody mimetic protein.

In one embodiment, the proteins or polypeptides that comprise the antibody or antigen binding portion thereof that binds to CD20 as described herein and a second moiety are fusion proteins. In one embodiment, the proteins or polypeptides that comprise the antibody or antigen binding portion thereof that binds to CD20 as described herein and a second moiety are drug conjugates.

As used herein “conjugate” refers to a composition comprising the antibody that binds to CD20 as described herein that is bonded/conjugated to a drug.

Such conjugates include “drug conjugates” which comprise antibody that binds to CD20 to which a drug is covalently bonded, and “non-covalent drug conjugates” which comprise the antibody that binds to CD20 min to which a drug is noncovalently bonded.

As used herein, “drug conjugate” refers to a composition comprising the antibody to which a drug is covalently bonded. The drug can be covalently bonded to the antibody fragment directly or indirectly through a suitable linker moiety. The drug can be bonded to the antibody at any suitable position, such as the amino-terminus, the carboxyl-terminus or through suitable amino acid side chains.

In one embodiment, the antibody is linked to the second moiety with a peptide linker or other suitable linker to connect the two moieties.

The term “peptide linker” refers to a peptide comprising one or more amino acids. A peptide linker comprises 1 to 50, for example 1 to 20 amino acids. Peptide linkers are known in the art and non-limiting examples are described herein. Suitable, non-immunogenic linker peptides are, for example, linkers that include G and/or S residues, (G4S)n, (SG4)n or G4(SG4)n peptide linkers, wherein “n” is generally a number between 1 and 10, e.g., 1, 2, 3, 4, 5, 6, 7, 8, 9 or 10.

The binding agent may be multispecific, for example bispecific.

In one embodiment, the binding molecule is bispecific. Thus, in one aspect, the invention relates to a bispecific molecule comprising an antibody described herein linked to a second moiety having a different binding specificity than said antibody. Thus, the second antibody binds to a different target antigen, e.g. a target of interest.

In one embodiment the binding molecule, e.g. the protein or construct is multispecific and comprises a further, i.e. third, fourth, fifth etc moiety.

The therapeutic moiety can also be selected from a half life extending moiety, cytotoxin, or radioisotope.

The non-therapeutic moiety can be selected from a label, liposome or nanoparticle. The label is detectable or functional. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

According to the invention, antibodies and antigen binding portion that is linked to one moiety may further be linked to another moiety. For example, a may be linked to a therapeutic moiety and further linkage to a non-therapeutic moiety may be provided either via the antibody or the moiety.

In one embodiment, the binding agent or the antibody or antigen binding portion thereof according to the invention may comprise a half life extending moiety. This may be selected from an antibody or antigen binding portion thereof that binds canine serum albumin. Alternatively, extended half life may be conferred through PEGylation.

The term “half-life” as used can generally refer to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art. The half-life can be expressed using parameters such as the tl/2-alpha, tl/2-beta and the area under the curve (AUC). Half-lives (t alpha and t beta) and AUC can be determined from a curve of serum concentration of conjugate or fusion against time. Thus, the term “half-life” as used herein in particular refers to the tl/2-beta or terminal half-life (in which the tl/2-alpha and/or the AUC or both may be kept out of considerations).

For example, in a first phase (the alpha phase) the drug composition (e.g., drug conjugate, noncovalent drug conjugate, drug fusion) is undergoing mainly distribution in the patient, with some elimination. A second phase (beta phase) is the terminal phase when the drug composition (e.g., drug conjugate, noncovalent drug conjugate, drug fusion) has been distributed and the serum concentration is decreasing as the drug composition is cleared from the patient. The t alpha half-life is the half-life of the first phase and the t beta half-life is the half-life of the second phase.

### Pharmaceutical Composition

In another aspect, there is provided a pharmaceutical composition comprising an antibody or fragment as described herein and optionally a pharmaceutically acceptable carrier. The term pharmaceutical composition as used herein refers to a composition that is used to treat a companion animal, that is for veterinary use, i.e. a veterinary composition. In preferred embodiments the animal that is treated is a dog.

The pharmaceutical composition may optionally comprise a pharmaceutically acceptable carrier. Antibodies, protein or construct or the pharmaceutical composition can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or sub-cutaneously. When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e.g. a gelatin capsule), it can contain, in addition to materials of the above type, a liquid carrier such as polyethylene glycol, cyclodextrin or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units. In specific embodiments, it can be desirable to administer the composition locally to the area in need of treatment, or by intravenous injection or infusion.

### Methods of Treating Disease

The invention further extends to methods for the treatment of a disease, administration of a pharmaceutical composition or formulation described herein or the antibody or antigen binding portion of the invention. Also envisaged is a pharmaceutical composition or formulation described herein or a binding molecule or fusion protein that comprises an antibody or antigen binding portion thereof as described herein for use in the treatment of disease.

In particular, the invention relates to a method of treating a condition mediated by B-cells in a canine subject in need thereof comprising administering an effective amount of the antibody or antigen-binding portion thereof as described herein.

An aspect of the invention is also an antibody or antigen-binding portion thereof or the pharmaceutical composition as described herein for use in the treatment of a condition mediated by B-cells in a canine subject.

For example, the antibody or antigen-binding portion thereof may be used to deplete canine blood and/or tissues of B cell lymphoma cells. The condition mediated by B-cells is selected from a B cell lymphoma, (e.g., diffuse large cell B cell lymphoma, Hodgkin's and non-Hodgkin's lymphoma, follicular lymphoma, mucosa-associated lymphatic tissue lymphoma (MALT), small cell lymphocytic lymphoma, chronic lymphocytic leukemia, mantel cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis), leukemia or an immune mediated disease. The immune mediated disease may be an autoimmune disease. Example may include, but are not limited to, autoimmune hemolytic anemia, immune-mediated thrombocytopenia, autoimmune blistering diseases, immune-mediated arthritis and atopic dermatitis, rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated hemolytic anemia and bullous pemphigoid.

The amount of the therapeutic that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody or fragment thereof of the present invention by weight of the composition. When intended for oral administration, this amount can be varied to range from about 0.1% to about 80% by weight of the composition. Preferred oral compositions can comprise from about 4% to about 50% of the antibody or fragment thereof of the present invention by weight of the composition.

Preferred compositions of the present invention are prepared so that a parenteral dosage unit contains from about 0.01% to about 2% by weight of the antibody or fragment thereof of the present invention.

For administration by injection, such as intravenous or sub-cutaneous injection, the composition can comprise from about typically about 0.01 mg/kg to about 250 mg/kg, for example 0.1 mg/kg to about 250 mg/kg of the subject's body weight, for example, between about 0.1 mg/kg and about 20 mg/kg of the animal's body weight, and more preferably about 1 mg/kg to about 10 mg/kg of the animal's body weight, although less than 0.1 mg/kg is also envisaged. In one embodiment, the composition is administered at a dose of about 0.5 to 30 mg/kg, e.g., about 5 to 25 mg/kg, about 10 to 20 mg/kg, about 0.5 to 5 mg/kg, about 0.5 to 2.5 mg/kg, about 0.5 to 2.0 mg/kg or about 2 or 3 mg/kg. In one embodiment, the composition is administered at a dose of 2 to 50 mg/ml. In one embodiment, the composition is administered at a dose of 0.5 mg/ml to 2.5 mg/ml, or 0.5 mg/ml to 5 mg/ml. The dosing schedule can vary from e.g., once a week to once every 2, 3, 4 weeks, or up to 8 weeks between doses. In one embodiment, the composition is administered at a dose of 0.5 mg/ml to 2.5 mg/ml every three to four weeks, e.g. 0.5 mg/ml or 2.5 mg/ml every three to four weeks. Suitably the dose is chosen so as to give prolonged depletion of CD20 positive cells to allow a three to four week interval between doses. Multiple doses may be administered, suitably up to about 6 or more repeat doses.

In one embodiment, post-treatment, the subject has at least 7 days, or at least 14 days, or at least 21 days, or at least 28 days, or at least 40 days, or at least 50 days, or at least 60 days disease progression-free. In one embodiment, post-treatment, the subject has at least 7 days, or at least 14 days, or at least 21 days, or at least 28 days, or at least 40 days, or at least 50 days, or at least 60 days disease progression-free.

In one embodiment, the number of days of survival, the number of disease free days, or the number of disease-progression free days is at least 2 months, or at least 3 months, or at least 4 months, e.g. at least 5 months, such as at least 6 months.

In one embodiment, the number of days of survival, the number of disease free days, or the number of disease-progression free days is at least 9 months, 200 days, 300 days or 3 years or more. In one embodiment, it is least one, two, three or more years. The invention provides methods of treating or preventing CD20-mediated diseases or disorders in a companion animal, e.g., a dog, comprising administering an effective amount of an antibody or fragment of the present invention to the animal in need thereof.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment. In treatment of B cell lymphomas, for example, ameliorating symptoms can be assessed by measuring lymph nodes after treatment and observing a reduction in lymph node size as an indication of successful treatment.

The term “subject” or “patient” refers to a dog, which is the object of treatment, observation, or experiment. For the avoidance of doubt, the treatment of humans is excluded.

The molecules or pharmaceutical composition of the invention may be administered as the sole active ingredient or in combination with one or more other therapeutic agent, for example a cancer therapy. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes, radioisotopes, immunosuppressant or an immunological modulating agent, such as a cytokine or a chemokine. In one example, the molecules or pharmaceutical composition of the invention may be administered in combination with a multi-agent, CHOP-based chemotherapy protocol incorporating several injectable and oral drugs (Lasparaginase, vincristine, Cytoxan, prednisone, and doxorubicin), given on a more-or-less weekly basis for a period of several months. Administration may be at the same time, prior or after administration of the compound of the invention.

The invention also relates to a method of inhibiting tumor growth or metastasis comprising contacting a tumor cell with an effective amount of the antibody or antigen-binding portion thereof or a pharmaceutical composition as described herein. The method can be in vitro, in vivo or ex vivo.

The invention also relates to a method of killing a tumor cell expressing CD20, comprising contacting the cell with an antibody or pharmaceutical composition as described herein, such that killing of the cell expressing CD20 occurs. The tumor cell is a canine tumor cell. The method can be in vitro, in vivo or ex vivo.

Methods for eliminating cells expressing canine CD20 using an antibody or pharmaceutical composition as described herein are also provided. The method can be in vitro, in vivo or ex vivo.

### Kit

In another aspect, the invention provides a kit for the treatment or prevention of a disease for example as listed herein or an immune response and/or for detecting CD20 for diagnosis, prognosis or monitoring disease comprising an antibody of the invention and optionally instructions for use. Such a kit may contain other components, packaging, instructions, or material to aid in the detection of CD20 protein. The kit may include a labelled antibody that binds to CD20 or a binding molecule comprising an antibody that binds to CD20 and one or more compounds for detecting the label.

### Methods of Making the Antibodies

An antibody described herein can be obtained from a transgenic mammal, for example a rodent, that expresses canine antibodies upon stimulation with an CD20 antigen. Such rodents are described in WO20018/189520 and WO2020/074874.

Thus, an antibody or fragment described herein can be obtained from a mammal, for example a rodent, for example a transgenic animal, that expresses antibodies upon stimulation with a canine CD20 antigen. The transgenic rodent, for example a mouse, may have a reduced capacity to express endogenous antibody genes. Thus, in one embodiment, the rodent has a reduced capacity to express endogenous light and/or heavy chain antibody genes. The rodent, for example a mouse, may therefore comprise modifications to disrupt expression of endogenous kappa and lambda light and/or heavy chain antibody genes so that no functional mouse light and/or heavy chains are produced, for example as further explained below. Such transgenic rodents are described in the art and this is further explained in the examples below.

Also within the scope of the invention is a method for producing canine antibodies capable of binding CD20 said method comprising
a) immunising a transgenic rodent, e.g. a mouse, with an CD20 antigen wherein said rodent expresses a nucleic acid construct comprising unrearranged canine V, D and J genes,
b) isolating canine antibodies.

Also within the scope of the invention is a method for producing antibodies capable of binding canine CD20 said method comprising
a) immunising a transgenic rodent, e.g. a mouse, with an CD20 antigen wherein said rodent expresses a nucleic acid construct comprising unrearranged canine V, D and J genes,
b) generating a library of sequences comprising heavy chain and light chain sequences from said rodent, e.g. a mouse and
c) isolating antibodies comprising heavy chain and light chain sequences from said libraries.

Further steps may include identifying an antibody that binds to CD20, for example by using functional assays as shown in the examples.

Methods for preparing or generating the polypeptides, nucleic acids, host cells, products and compositions described herein using in vitro expression libraries can comprise the steps of:
a) providing a set, collection or library of nucleic acid sequences encoding amino acid sequences; and
b) screening said set, collection or library for amino acid sequences that can bind to/have affinity for CD20 and
c) isolating the amino acid sequence(s) that can bind to/have affinity for CD20

In the above method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty). Libraries, for example phage libraries, are generated by isolating a cell or tissue expressing an antigen-specific antibody or fragment thereof, cloning the sequence encoding the antibody or fragment thereof mRNA derived from the isolated cell or tissue and displaying the encoded protein using a library. The sequences can be expressed in bacterial, yeast or other expression systems.

Another aspect also relates to an isolated antibody obtained or obtainable by a method described above.

### Other Methods and Uses

In another aspect, the antibody or antigen-binding portion thereof as described herein is used for non-therapeutic purposes, such as diagnostic tests and assays. The invention thus also relates to a method for detecting a canine cell expressing canine CD20 or detecting a canine CD20 protein in a biological sample from a canine subject, comprising contacting a biological sample with the antibody or antigen-binding portion thereof as described herein wherein said antibody or antigen-binding portion thereof is linked to a detectable label. The biological sample may be a biopsy, tissue, blood, serum, plasma, or lymphatic fluid sample.

In certain embodiments, the method may include comparing the amount of binding in the test biological sample to the amount of binding in a control biological sample, wherein increased binding to the test biological sample relative to the control biological sample may indicate the presence of one or more lymphoma cells in the test biological sample. In some embodiments, the biological sample is canine blood or a needle aspirate. These methods are also provided in an in vivo and/or in vitro format.

Modifications of antibodies for diagnostic purposes are well known in the art. For example, antibodies may be modified with a ligand group such as biotin, or a detectable marker group such as a fluorescent group, a radioisotope, or an enzyme. Compounds of the invention can be used for diagnostic purposes and e.g. labelled using conventional techniques. Suitable detectable labels include but are not limited to fluorophores, chromophores, radioactive atoms, electron-dense reagents, enzymes, and ligands having specific binding partners.

In another aspect, the antibody or antigen-binding portion thereof as described herein is used in the isolation and/or identification of cells expressing canine CD20 or cells that contain a cell surface protein that reacts with these binding agents (e.g., B cells, B lymphoma cells, canine CD20).

The antibody or antigen-binding portion thereof as described herein can also be used in an assay to determine the level of CD20 expression. The level of expression may then be correlated with base (e.g., control) levels to determine whether a particular disease is present within the patient, the patient's prognosis, or whether a particular treatment regimen is effective.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further illustrated in the following non-limiting examples.

### EXAMPLES

### Example 1: Cloning Canine CD20

A search of the CAMFAM_3.1 boxer reference genome was conducted using the UCSC Genome Browser. The genomic sequence for CD20 (MS4A1) was downloaded along with mRNA sequence AB210085.1. Using this sequence data, primers were designed to enable amplification of CD20 from cDNA with added sequence to allow for seamless cloning. This allowed for confirmation of the CD20 sequence in dog blood and seamless cloning into a piggyBac cloning vector.

Isolation of Canine CD20 mRNA and Generation of cDNA

Beagle whole blood was supplied by Envigo RMS (Alconbury, Huntingdon, UK) and PBMCs were isolated using a Ficoll gradient. Briefly, 10 ml whole blood was diluted with 25 ml phosphate buffered saline (PBS) and layered onto 15 ml Ficoll Paque Plus (Sigma Aldrich) before centrifuging at 800rcf for 10 min, room temp, with slow acceleration and no brake. The interphase disk was collected into PBS. Total RNA was isolated from PBMCs with the QIAGEN RNeasy Mini Kit (Qiagen, Hilden, DE) and standard procedures, with an on-column DNAse digestion. cDNA generation was undertaken using the SuperScript™ IV First-Strand Synthesis System following standard procedures and anchored oligo dT primers (ThermoFisher, Massachusetts, US).

The nucleotide and amino acid sequence of full length canine CD20 for cellular expression is shown below:

NUCLEOTIDE:

(SEQ ID NO: 1)
ATGACAACACCCAGAAATTCAATGAGTGGAACTCTCCCGGTAGATCCTA
TGAAAAGCCCTACTGCCATGTATCCTGTTCAAAAAATAATTCCCAAAAG
GATGCCTTCAGTGGTGGGCCCTACACAAAACTTCTTCATGAGGGAATCT
AAGACACTGGGGGCTGTCCAGATTATGAATGGGCTCTTCCACATTGCCC
TAGGCAGCCTCCTGATGATTCACACGGATGTCTATGCGCCCATCTGTAT
AACTATGTGGTACCCTCTCTGGGGAGGCATTATGTTCATCATTTCTGGA
TCACTCCTGGCAGCAGCGGACAAAAACCCCAGGAAGAGTTTGGTCAAAG
GAAAAATGATAATGAACTCATTGAGCCTCTTTGCTGCTATTTCTGGAAT
AATTTTTTTGATCATGGACATATTTAATATTACCATTTCCCATTTTTTT
AAAATGGAGAATTTGAATCTTATTAAAGCTCCCATGCCATATGTTGACA
TACACAACTGTGACCCAGCTAACCCCTCTGAGAAAAACTCTTTATCTAT
ACAATATTGTGGCAGCATACGATCTGTTTTCTTGGGCGTTTTTGCTGTG
ATGGTGATCTTTACCTTTTTCCAGAAACTTGTGACAGCTGGCATTGTTG
AGAATGAATGGAAAAAACTGTGCTCTAAACCTAAATCTGATGTAGTTGT
TCTGTTAGCTGCTGAAGAAAAAAAAGAACAGCCGATTGAAACAACAGAA
GAAATGGTTGAGCTGACTGAAATAGCTTCCCAACCAAAGAAAGAAGAAG
ACATTGAAATTATTCCAGTCCAAGAAGAAGAAGAGGAACTGGAAATAAA
CTTTGCAGAACCTCCCCAGGAGCAGGAATCTTCACCAATAGAAAACGAC
AGCATCCCTTAA

PROTEIN:

(SEQ ID NO: 2)
MTTPRNSMSGTLPVDPMKSPTAMYPVQKIIPKRMPSVVGPTQNFFMRES
KTLGAVQIMNGLFHIALGSLLMIHTDVYAPICITMWYPLWGGIMFIISG
SLLAAADKNPRKSLVKGKMIMNSLSLFAAISGIIFLIMDIFNITISHFF
KMENLNLIKAPMPYVDIHNCDPANPSEKNSLSIQYCGSIRSVFLGVFAV
MVIFTFFQKLVTAGIVENEWKKLCSKPKSDVVVLLAAEEKKEQPIETTE
EMVELTEIASQPKKEEDIEIIPVQEEEEELEINFAEPPQEQESSPIEND
SIP

### Example 2: Expressing Canine CD20

Human embryonic kidney (HEK) 293 cells were grown on 90 mm round tissue culture plates as monolayers in DMEM/F12 (Life Technologies) supplemented with 10% fetal bovine serum (FBS; Sigma Aldrich) at 37° C., with 5% CO₂. HEK293 cells were co-transfected with CD20 cDNA and PiggyBac transposase using polyethyleneimine (PEI MAX: 40 kDa, Polysciences Inc., Eppelheim, Germany). 30 μl of PEI MAX (1 mg ml⁻¹), 5 μg cDNA and 1 ml DMEM/F12 were incubated for 10 min at room temperature, added dropwise to a 90 mm plate of 70-80% confluent HEK293 cells, and incubated for 2 days before use. Stably transfected cells were selected 48 hrs later using a suitable antibiotic.

Mouse embryonic fibroblasts (MEF) were grown on 90 mm round tissue culture plates as monolayers in DMEM-high glucose (Life Technologies) supplemented with 10% FBS, 1 mM Sodium pyruvate (Sigma-Aldrich), 0.5 mM p-mercaptoethanol (Gibco) and 1% MEM non-essential amino acids (Sigma-Aldrich) at 37° C., with 5% CO₂. Cells were transfected with CD20 cDNA and PiggyBac transposase using Lipofectamine LTX with PLUS™ reagent (ThermoFisher Scientific) according to the manufacturer's recommended instructions. Stably transfected cells were selected 48 hrs later using a suitable antibiotic.

MDCK II (Madin-Darby canine kidney) cells were grown on surface treated tissue culture flasks (T25/T75/T175) as monolayers in DMEM-high glucose (Life Technologies) supplemented with 10% FBS, and 1% MEM non-essential amino acids (Sigma-Aldrich) at 37° C., with 5% CO2. MDCK II cells were co-transfected with wild-type or mutant canine CD20 cDNA and piggyBac transposase using Lipofectamine™ LTX reagent with PLUS™ reagent (ThermoFisher Scientific) by following the recommended protocol.

### Example 3: Immunisation of Mouse Model Using DNA and MEF

Ky9™ mice, substantially as described in WO2018/189520 and WO2020/074874, for example, were immunised. The transgenic mice have been modified by insertion of the dog immunoglobulin variable gene repertoire into the corresponding loci of the mouse genome. This allows for the production of antibodies that comprise a variable antibody region originating from the expression of canine DNA in the mouse, in combination with a mouse constant region (for heavy chain and kappa chain) or a dog constant region (for lambda chain). Information concerning, or the nucleic acid comprising, the variable region of such chimeric antibody chains may be used to generate fully canine antibodies.

For DNA immunisation, prime and boost regime using hydrodynamic tail vein injection (HTVI) was performed and tissues harvested. For cell-based immunisation, a prime and boost regime using MEF cells stably expressing CD20 was performed and tissues harvested. A further immunisation regime was performed using HTVI DNA immunisation for the prime combined with CD20-expressing MEF cell boosters.

Determination of serum titres:

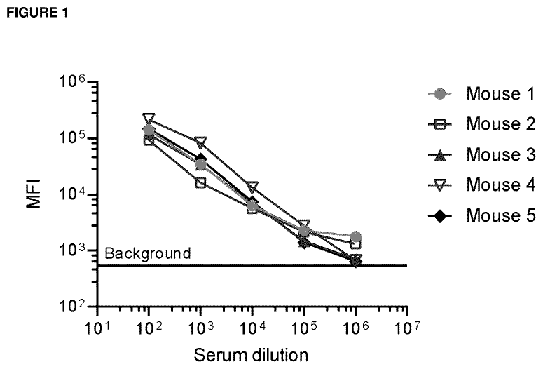

Mice were bled prior to immunisation and 10 days after each subsequent boost. Sera were separated from clotted blood by centrifugation in microvette 200 Z-gel tubes (Starstedt AG & Co. KG, Germany) and antibody titres against canine CD20 were evaluated by flow cytometry. Sera were serially diluted 1:10 in FACS buffer (PBS+3% FBS) and added to cells stably-expressing canine CD20, or canine CD20 negative control cells. Mouse antibodies to canine CD20 were detected with either BB700 conjugated (BD Horizon Brilliant™ Blue 700, BD Biosciences) or FITC-conjugated secondary monoclonal antibodies against isotypes IgG1, IgG2a, IgG2b (BD OptiBuild™, Becton Dickinson). Data were acquired using a BD Accuri C6 Flow Cytometer (Becton Dickinson, NJ, USA) or Beckman Coulter CytoFLEX. The pre-immunisation sera were used to determine background. Antibody titres were determined as the highest dilution that showed positive signal above background. FIG. 1 shows antibody titres of 5 immunised Ky9 mice.

### Example 4: Isolation of Antibody Producing Cells, Antibody Sequencing, Selection and Sequence Modification

Tissue isolation: Spleens, lymph nodes and bone marrow were harvested from mice. Splenocytes were prepared by cutting the spleen into pieces and mashing them through a 40 μm cell strainer (Falcon) while rinsing with RPMI-1640 (Lonza, Basel, CH)+10% FBS on ice. A similar process was used for lymphocytes from lymph nodes with spleen and lymph node cells generally being pooled. Bone marrow was collected from femur and tibia by flushing the marrow with RPMI-1640 using a 25-gauge needle, through a 40 μm cell strainer pre-wetted with RPMI-1640. All cell types were pelleted at 300 g for 5 min and either directly used for flow sorting or resuspended in FBS+10% dimethyl sulfoxide (DMSO) before being frozen at −150° C.

Cell Sorting: Generally, antigen-specific splenic B cells can be captured by labelled antigen protein probes (eg. extra cellular domain) or antigen-VLPs because they dominantly express transmembrane antibodies on the cell surface. On the other hand, antigen-specific plasmablast or plasma cells are thought to be less easily labelled by protein probes or VLPs because of their dominant expression of secreted antibodies. Therefore, plasmablasts and plasma cells isolated from the spleen lymph nodes sample or bone marrow were proceeded to the next step of antibody sequence recovery without using antigen probes to isolate the antigen-specific subset of these populations. Cell surface co-expression of CD138 and CD267 (TACI) was used to identify the population of plasmablasts and plasma cells. For bulk sorting of plasmablasts and plasma cells for 10X Genomics Chromium Single Cell Immune Profiling, a CD138 plasma cell enrichment kit (Miltenyi biotech UK) was used to enrich these rare cells. Prior to antigen-specific cell sorting from spleen and lymph node cells, B cell enrichment was undertaken using a mouse pan-B cell isolation kit according to the manufacturer's instructions (StemCell Technologies UK) or using an in-house biotinylated antibody cocktail and streptavidin rapidspheres (StemCell Technologies UK) according to the manufacturer's instructions.

Markers including CD19, IgM, IgA, IgD, CD138 and CD267 (TACI) are then used to identify isotype-switched B cells enriched in cells that are responding to the immunisation. Within this population, antigen-specific cells can be captured by staining with labelled protein probes or VLPs that express target antigen upon their surface and flow sorting. VLPs are generated from HEK cells stably transfected with CD20, and the retrovirus gag protein fused to EGFP; the gag expression enables VLP budding from cells, and EGFP labels the VLPs for fluorescence-detection. Surface antigens on VLPs are directly expressed from recombinant cells without any step of purification or modification, and are presented in a native form. Other mammalian cell lines, such as Chinese Hamster Ovary cells (CHO) or mouse embryonic fibroblasts (MEF) can also be used for VLP production. Markers to identify unwanted cell populations and dead cells (F4/80; Ly-6C/G; CD8a; CD4; CD11c; 7AAD or zombie-NIR/FVD EF780) are included in all staining panels to exclude these cells from the sorting procedure.

Next Generation Sequencing and candidate selection:

Sorted cells are prepared for antibody profiling using the 10X Genomics Chromium Single Cell Immune Profiling system and the V(D)J Kit (10X Genomics) according to the manufacturer's instructions. For 10X, various Illumina platform instruments are used according to the instructions from 10X Genomics. The sequences are analysed using custom tools based on the pRESTO/Change-O (Yale University)/IgBlast (NCBI, USA)/Enclone (10X Genomics) software to predict the germline sequence and hypermutation.

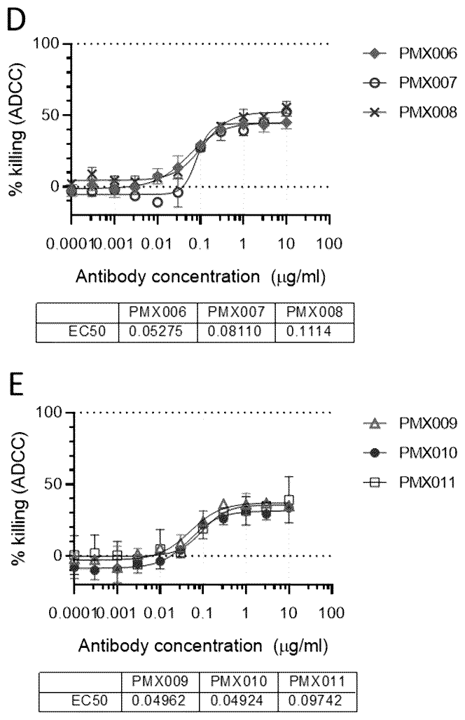

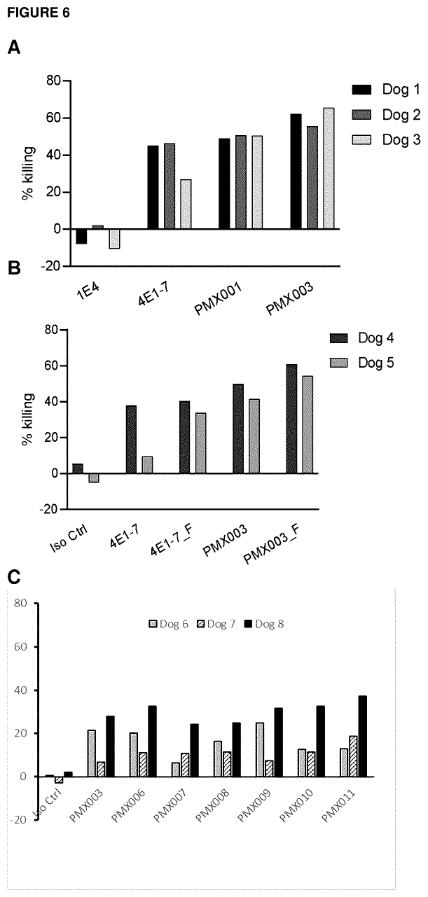

The variable immunoglobulin region comprises a VDJ region of an immunoglobulin nucleotide sequence for heavy genes and a VJ region of an immunoglobulin nucleotide sequence for IgK and IgA. Within a clonal family there are subfamilies with shared mutations within their V(D)J segments that arise during immunoglobulin gene recombination and somatic hypermutation. Different clonal families that display unique V(D)J segment usage usually exhibit different binding characteristics. During recombination and hypermutation, cells whose antibodies have a higher affinity for an antigen are selected. The affinity usually increases with further mutations; for example, a clustered family is shown in FIG. 6 of WO2015/040401.

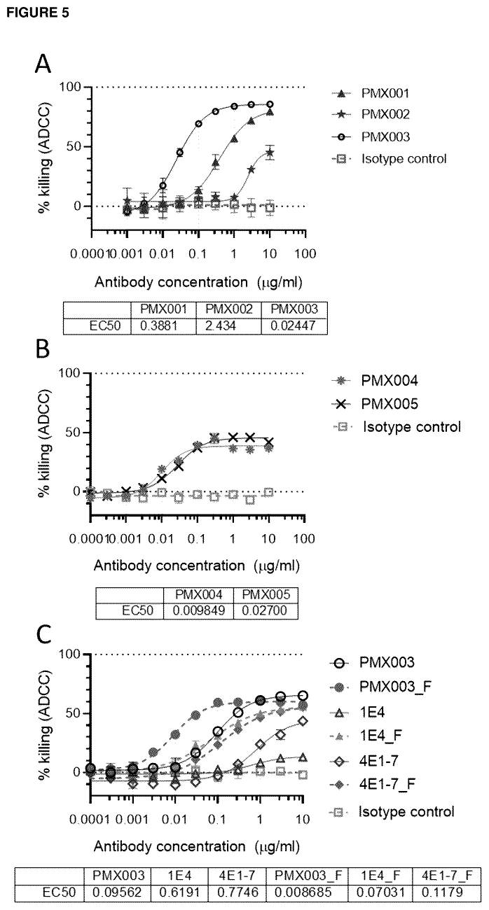

A clonal family is generally defined by related immunoglobulin heavy chain and light chain V(D)J sequences of two or more clonal cells. Related immunoglobulin V(D)J sequences can be identified by their shared usage of V and J gene segments. An example of the analysis of antibody sequences of sorted Ag-specific single B-cells is shown in FIG. 5 of WO2015/040401, and shows antibody sequences that are arranged by heavy-chain V-gene family usage, and clustered to generate the displayed phylogenetic trees. From phylogenetic trees such as these, candidate clones are selected.

For instance, anti-canine CD20 mAbs PMX001, PMX002, PMX003, PMX004 and PMX005 are all encoded by the same heavy chain V-gene (cIGHV3-5), heavy chain J-gene (cIGHJ4, with germline sequence given in SEQ ID NO: 75), light chain V-gene (cIGLV3-3) and light chain J-gene (cIGLJ3, with germline sequence given in SEQ ID NO: 77). PMX001 variable region sequences were first identified from a canine CD20 immunised Ky9 mouse. It has a heavy chain variable region nucleotide sequence (SEQ ID NO: 3) that encodes for the corresponding amino acid sequence (SEQ ID NO: 4) and a light chain variable region nucleotide sequence (SEQ ID NO: 5) that encodes for the corresponding amino acid sequence (SEQ ID NO: 6). There are 2 amino acid changes in the heavy chain variable sequence as compared to germline sequence (SEQ ID NO: 74) and 3 amino acid changes in light chain variable sequence as compared to the germline sequence (SEQ ID NO: 76). The CDR1 (SEQ ID NO: 7), CDR2 (SEQ ID NO: 8) and CDR3 (SEQ ID NO: 9) of the heavy chain variable region and the CDR1 (SEQ ID NO: 10), CDR2 (SEQ ID NO: 11) and CDR3 (SEQ ID NO: 12) of the light chain variable region are predicted using IMGT V-QUEST (http://www.imgt.org/lMGT_vquest/input). The CDR3 regions of the heavy chain and the light chain of PMX mAbs are Saa and 9aa in length, respectively.

Hit expansion of PMX001 mAb was later performed in all the canine CD20 immunised mice, and candidates that share the same VJ gene usage and CDR3 length for both heavy and light chains were selected for further screening. Further mutations may occur in the sequences compared to PMX001 mAb. For instance, PMX002, PMX003, PMX004 and PMX005 mAbs were selected from other immunisation cohorts and hence different mice from where PMX001 mAb was identified. It shows the convergent selection of successful gene rearrangement.

PMX002 mAb has a heavy chain variable region nucleotide sequence (SEQ ID NO: 13) that encodes for the corresponding amino acid sequence (SEQ ID NO: 14) and a light chain variable region nucleotide sequence (SEQ ID NO: 15) that encodes for the corresponding amino acid sequence (SEQ ID NO: 16). There are 10 amino acid changes in the heavy chain variable sequence as compared to germline sequence (SEQ ID NO: 74) and no amino acid change in light chain variable sequence (SEQ ID NO: 76). The CDR1 (SEQ ID NO: 17), CDR2 (SEQ ID NO: 18) and CDR3 (SEQ ID NO: 19) of the heavy chain variable region and the CDR1 (SEQ ID NO: 20), CDR2 (SEQ ID NO: 21) and CDR3 (SEQ ID NO: 22) of the light chain variable region are predicted using IMGT V-QUEST.

PMX003mAb has a heavy chain variable region nucleotide sequence (SEQ ID NO: 23) that encodes for the corresponding amino acid sequence (SEQ ID NO: 24) and a light chain variable region nucleotide sequence (SEQ ID NO: 25) that encodes for the corresponding amino acid sequence (SEQ ID NO: 26). There are 5 amino acid changes in the heavy chain variable sequence as compared to germline sequence (SEQ ID NO: 74) and 1 amino acid change in light chain variable sequence (SEQ ID NO: 76). The CDR1 (SEQ ID NO: 27), CDR2 (SEQ ID NO: 28) and CDR3 (SEQ ID NO: 29) of the heavy chain variable region and the CDR1 (SEQ ID NO: 30), CDR2 (SEQ ID NO: 31) and CDR3 (SEQ ID NO: 32) of the light chain variable region are predicted using IMGT V-QUEST.

PMX004 mAb has a heavy chain variable region nucleotide sequence (SEQ ID NO: 33) that encodes for the corresponding amino acid sequence (SEQ ID NO: 34) and a light chain variable region nucleotide sequence (SEQ ID NO: 35) that encodes for the corresponding amino acid sequence (SEQ ID NO: 36). There are 12 amino acid changes in the heavy chain variable sequence as compared to germline sequence (SEQ ID NO: 74) and no amino acid change in light chain variable sequence (SEQ ID NO: 76). The CDR1 (SEQ ID NO: 37), CDR2 (SEQ ID NO: 38) and CDR3 (SEQ ID NO: 39) of the heavy chain variable region and the CDR1 (SEQ ID NO: 40), CDR2 (SEQ ID NO: 41) and CDR3 (SEQ ID NO: 42) of the light chain variable region are predicted using IMGT V-QUEST.

PMX005 mAb has a heavy chain variable region nucleotide sequence (SEQ ID NO: 43) that encodes for the corresponding amino acid sequence (SEQ ID NO: 44) and a light chain variable region nucleotide sequence (SEQ ID NO: 45) that encodes for the corresponding amino acid sequence (SEQ ID NO: 46). There are 4 amino acid changes in the heavy chain variable sequence as compared to germline sequence (SEQ ID NO: 74) and no amino acid change in light chain variable sequence (SEQ ID NO: 76). The CDR1 (SEQ ID NO: 47), CDR2 (SEQ ID NO: 48) and CDR3 (SEQ ID NO: 49) of the heavy chain variable region and the CDR1 (SEQ ID NO: 50), CDR2 (SEQ ID NO: 51) and CDR3 (SEQ ID NO: 52) of the light chain variable region are predicted using IMGT V-QUEST.

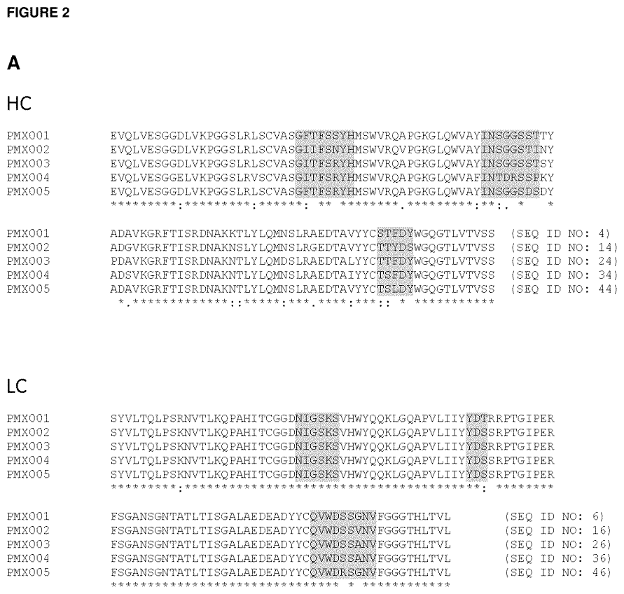

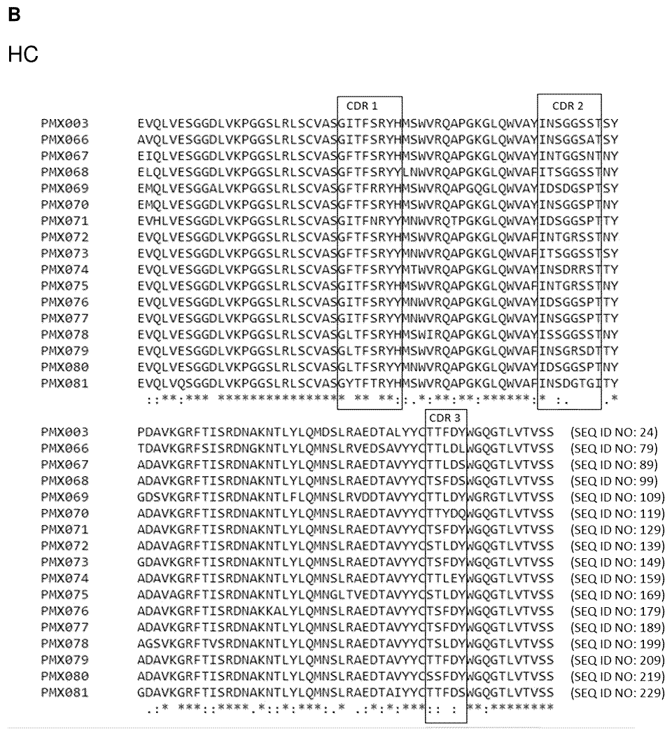

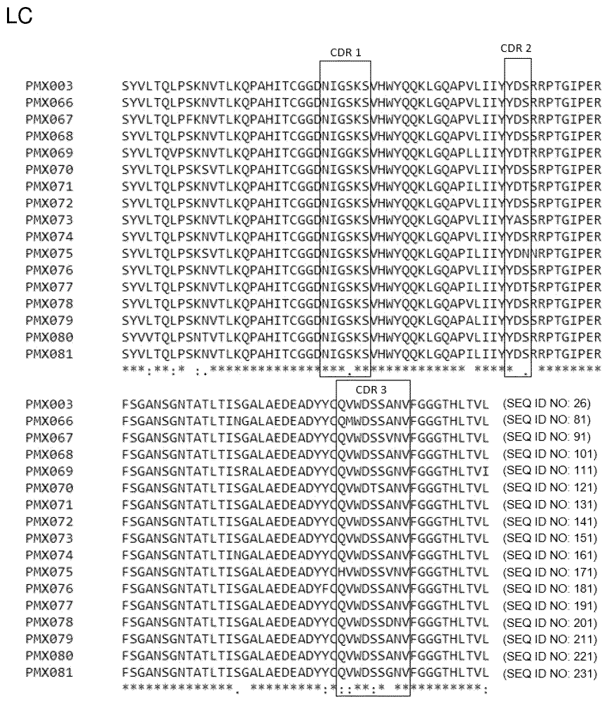

FIG. 2 shows an alignment of PMX antibody sequences.

### Sequence Modification

The framework region 1 (FR1) of the light chain of PMX003 (SEQ ID NO: 26) contains the NVT sequon, which is a consensus sequence for asparagine-linked glycosylation of proteins. To remove the glycans bound to this site, the NVT sequon in the FR1 of PMX003 mAb was mutated to QVT for PMX006 mAb, AVT for PMX007 mAb, EVT for PMX008 mAb, NVA for PMX009 mAb, SVT for PMX010 mAb and TVT for PMX011 mAb.

The resulted light chain variable region amino acid sequences of PMX006 mAb (SEQ ID NO: 53), PMX007 mAb (SEQ ID NO: 54), PMX008 mAb (SEQ ID NO: 55), PMX009 mAb (SEQ ID NO: 56), PMX010 mAb (SEQ ID NO: 57), PMX011 mAb (SEQ ID NO: 58) and the FR1 sequences of PMX003 (SEQ ID NO: 59), PMX006 mAb (SEQ ID NO: 60), PMX007 mAb (SEQ ID NO: 61), PMX008 mAb (SEQ ID NO: 62), PMX009 mAb (SEQ ID NO: 63), PMX010 mAb (SEQ ID NO: 64), PMX011 mAb (SEQ ID NO: 65) are listed in the Sequences Table.

The framework region 1 (FR1) of the light chain of PMX066 (SEQ ID NO: 81), PMX0067 (SEQ ID NO: 91), PMX068 (SEQ ID NO: 101), PMX0069 (SEQ ID NO: 111), PMX071 (SEQ ID NO: 131), PMX072 (SEQ ID NO: 141), PMX073 (SEQ ID NO: 151), PMX074 (SEQ ID NO: 161), PMX076 (SEQ ID NO: 181), PMX077 (SEQ ID NO: 191), PMX078 (SEQ ID NO: 201), PMX079 (SEQ ID NO: 211), PMX081 (SEQ ID NO: 231), contains the NVT sequon. To remove the glycans bound to this site, the NVT sequon in the FR1 of PMX066, PMX0067, PMX068, PMX0069, PMX071, PMX072, PMX073, PMX074, PMX076, PMX077, PMX078, PMX079, PMX081 mAb was mutated to SVT to generate the resulting light chain variable region amino acid sequences PMX112 (SEQ ID NO: 238), PMX113 (SEQ ID NO: 239), PMX114 (SEQ ID NO: 240), PMX115 (SEQ ID NO: 241), PMX116 (SEQ ID NO: 242), PMX117 (SEQ ID NO: 243), PMX118 (SEQ ID NO: 244), PMX119 (SEQ ID NO: 245), PMX120 (SEQ ID NO: 246), PMX121 (SEQ ID NO: 247), PMX122 (SEQ ID NO: 248), PMX123 (SEQ ID NO: 249) and PMX124 (SEQ ID NO: 250) respectively. The sequences are listed in Table 2. A number of the antibodies comprise naturally occurring sequence motifs which do not result in glycan being bound to the antibody. For example, PMX070 and PMX075 comprise the sequence motif SVT in FR1 of the light chain, PMX080 comprises the sequence motif TVT in FR1 of the light chain.

### Example 5: Generation of Monoclonal Antibodies from Single Cells

The heavy chain and light chain V(D)J sequence of selected candidate clones are synthesised and cloned into expression vectors containing the genomic sequences of the dog IgG constant region, and the dog IGK or IGL constant regions, respectively. For instance, PMX001 to PMX005 variable sequences were cloned into the vectors that encode the dog IGGB (SEQ ID NO: 66) and dog IGLC5 constant regions (SEQ ID NO: 67). The expression vectors encoding the heavy chain and light chain were co-transfected into a suitable mammalian cell line such as CHO cells to obtain stable expression. Fully canine sequence antibodies were thus generated.

To facilitate the expression in CHO cells, codon-optimisation can be performed, for example, on the nucleotide sequences of the heavy chain variable region (SEQ ID NO: 68) and light chain variable region (SEQ ID NO: 69) of PMX003 mAb. Campath leader (SEQ ID NO: 70) (see U.S. Pat. No. 8,362,208 B2)| can be introduced to replace the native leader (SEQ ID NO: 71 for heavy chain, SEQ ID NO: 72 for light chain) of PMX001 to PMX005 mAbs.

Monoclonal antibodies 1E4 and 4E1-7 were also expressed. 1E4 nucleotide sequence was obtained from WO2013063186. 4E1-7 amino acid sequence was obtained from Mizuno et al. Scientific Reports, 10, Article 11476 (2020) (https://doi.org/10.1038/s41598-020-68470-9). The variable regions of both antibodies were cloned into vectors that encode the dog IGGB (SEQ ID NO: 66) and dog IGK (SEQ ID NO: 73) constant regions. Rituximab-clGGB control mAb was generated by synthesizing the variable region sequences (see U.S. Pat. No. 5,736,137) and cloned into vectors that encode dog IGGB (SEQ ID NO: 66) and dog IGLC5 (SEQ ID NO: 67) constant regions.

The core fucose on the N-linked glycans bound to the Fc portion of antibodies could affect the binding of Fc receptors to the Fc region of the antibodies and therefore reduce the ADCC activities. To enhance the ADCC killing activities, Fut8 knockout (KO) CHO cells were generated in a pooled format, by deleting exon 2 of Fut8 coding gene as described in Yamane-Ohnuki et al., Biotechnol Bioeng. 2004 Sep. 5; 87(5):614-22. https://pubmed.ncbi.nlm.nih.gov/15352059/). The KO cells were phenotypically selected using LCA staining.

For antibody production, 6×10⁶ selected CHO cells or Fut8 knockout CHO cells were seeded in 3 ml culture media and incubated at 32° C., 8% CO₂ with shaking at 200 rpm. 4% HyClone Cell Boost 7a supplement+0.4% HyClone Cell Boost 7b supplement+1% glucose was added to the media on days 1, 4, 7 and 10. Culture supernatants were collected on day 12 and the IgG concentration was determined using surface plasmon resonance (Biacore 8K, Cytiva Life Sciences).

### Example 6: Binding Assays

Flow cytometry-based assay:

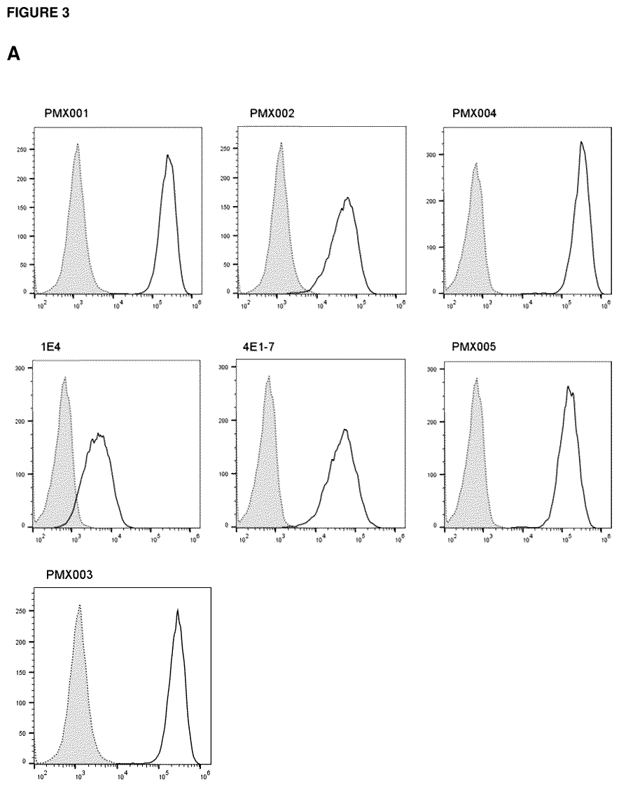

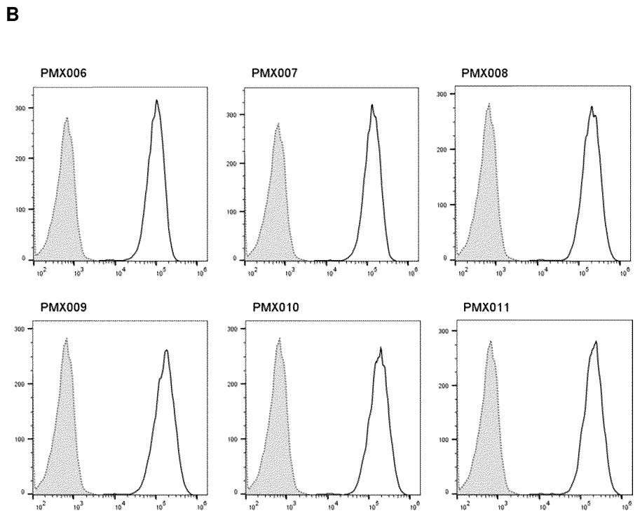

HEK293 cells or MDCK II cells were stably transfected with a vector encoding the full length of canine CD20 cDNA. Binding of antibodies to these cells were assessed using flow cytometry. In brief, 1-2×10⁵ canine CD20-expressing cells were incubated with candidate mAbs for 1 hour at +4° C., at a fixed concentration of 1 μg/ml or 10 μg/ml for binding assays and a range of concentrations (12-point 1:2 serial dilution antibodies starting at 30 μg/ml i.e. 200 nM) for affinity determination, followed by incubation with 5 μg/ml of FITC-conjugated anti-canine IgG secondary antibody (Bethyl Laboratories) for 1 hour at +4° C. Cells incubated with anti-canine IgG FITC secondary antibody and without primary anti-canine CD20 antibody, or with isotype control primary antibody, were used as negative controls. The data were acquired on either a Beckman Coulter CytoFLEX or a BD Accuri C6 Plus flow cytometer and analysed using FlowJo software. For affinity determination, stained cells were washed and fixed with 1% paraformaldehyde/3% FBS/PBS for 15 min after incubation with candidate antibodies and were fixed again for 24 h after incubation with secondary antibody before data acquisition on the flow cytometer. Graphs using mean fluorescence intensity (MFI) values of the FITC channel vs concentration of antibody were plotted in GraphPad Prism (FIG. 3D). Apparent affinity (Kd) of binding of candidate antibodies to cell surface canine CD20 was determined as the concentration when 50% of CD20-expressing cells were stained, i.e. EC50 of binding, using an equation for log(agonist) vs response—variable slope (four parameters).

Results of binding assays are shown in FIG. 3 A-C. These show that PMX antibodies had a stronger binding capacity to canine CD20 than test antibodies 1 E4 and 4E1-7 at a single point of concentration (10 ug/ml), apart from PMX002 which was equivalent to 4E1-7 antibody.

ELISA-based assay:

The large loop of the extracellular domain (ECD) of canine CD20 protein, as described below, is expressed in CHO cells and secreted into the extracellular media under the control of the CAG promoter, before purification using a protein A column.

CD20 ECD protein is coated to the assay plate. Antibodies are added at a range of concentrations and the binding capacity determined using ELISA.

SPR-based assay:

Affinity (K_{d}) of anti-canine CD20 mAbs can be measured by SPR, using a recombinant mouse Fc tagged CD20 extracellular domain.

The recombinant extracellular domain (ECD) (large loop) of CD20 named dCD20LL-Fc (see WO2013/063186, page 37, SEQ ID NO:62) is expressed in secreted form with a murine IgG2a Fc tag from stable CHO cells and purified from clarified supernatant using protein A chromatography. An apparent K_{d} corresponding to the bivalent avidity of the interaction is derived by amine coupling 100-150 RU of dCD20LL-Fc onto flow cell 2 of a CM5 chip with NHS/EDC activation/deactivation applied to flow cell 1. Three-fold dilutions from 200 nM to 0.1 nM concentrations of candidate IgG are made in HBS-EP+ buffer and injected over the chip surface for 180 s at 30 μL/min. Dissociation is monitored for 600 s at 30 μL/min before regenerating the surface with a 60 s pulse of 10 mM glycine-HCl pH 2.0. A monovalent K_{d} corresponding to the monovalent affinity is derived by repeating the protocol above using candidate antibody Fab fragment generated by papain (or SpeB) digestion of the full-length IgG. Experiments are conducted in HBS-EP+ running buffer at 25° C. All SPR experiments are carried out on a Biacore 8K instrument and data analysed by fitting the data to a 1:1 binding model using Biacore Insight Evaluation software.

### Example 7: Functional Assays

### Complement Dependent Cytotoxicity (CDC) Activity

CLBL-1 canine lymphoma tumour cell line (University of Veterinary Medicine Vienna) that natively expresses canine CD20 was used as the target cell line for a CDC assay. 10,000 CLBL-1 cells per well of 96-well plate (white with clear bottom) were incubated with a titration (11-point 1:3 dilution starting at 10 μg/ml) of anti-canine CD20 antibody and canine complement preserved serum (BioIVT) at a final dilution of 1:4, for 2 hours at 37° C., 5% CO2. The assay was set up using media (RPMI+1% L-glutamine+20% fetal bovine serum) made using heat inactivated serum so that canine complement preserved serum would be the only source of complement. Rituximab-clGGB chimeric antibody was used as the negative, isotype control.

Live cells were then quantified using CellTitre-Glo® Luminescent Cell Viability Assay (Promega) following the assay protocol. This assay uses the ATP content of live cells as an indication of cell viability. Luminescence was measured on a CLARIOstar (BMG Labtech). Data were analyzed using MARS software (BMG Labtech) and the number of live cells remaining was used to calculate the percentage of killing in the presence of antibodies using Microsoft Excel, using wells without antibody as baseline. Graphs were plotted in GraphPad Prism.

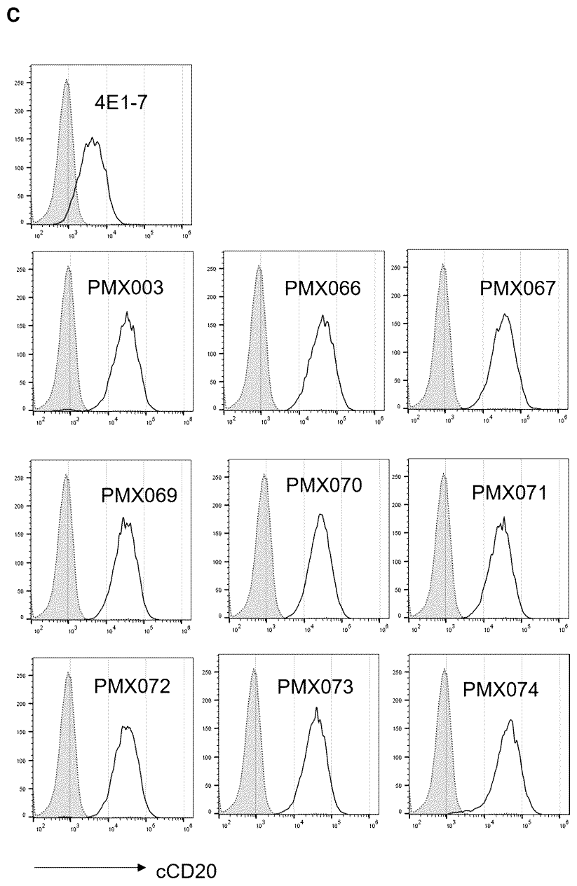

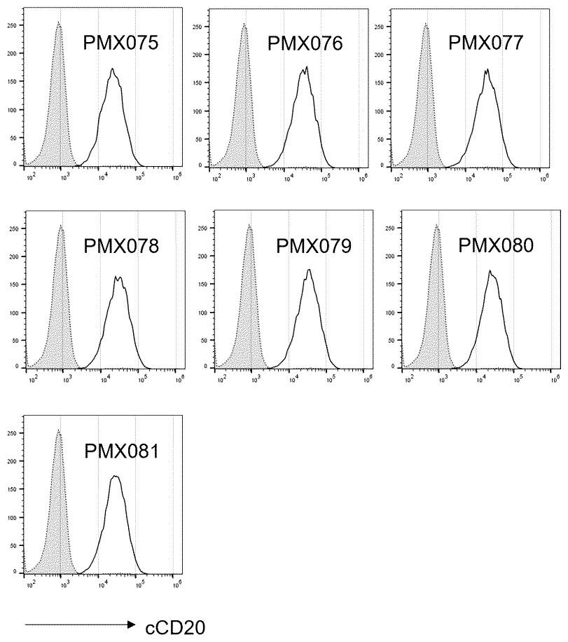

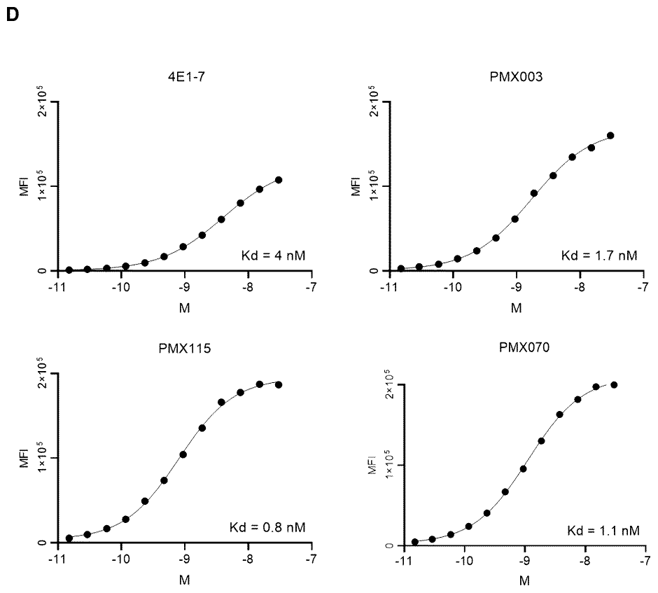

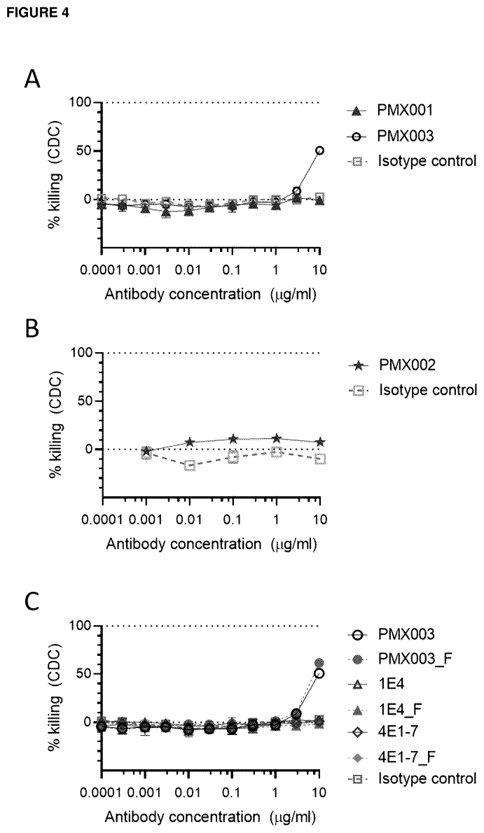

FIG. 4 shows CDC activity was observed with PMX003 in both native and a-fucosylated form. No CDC activity was observed for the native and a-fucosylated form of 1 E4 and 4E1-7 test antibodies under the same experimental settings using canine complement preserved serum. Table 4 summarises the CDC activity for antibodies PMX066 to PMX081.

### Antibody Dependent Cellular Cytotoxicity (ADCC) Activity

Canine cell line, such as MDCK II cell line (ATCC), was stably transfected with a construct encoding for the canine CD20 protein and a construct expressing a fluorescent protein (e.g. GFP). Either MDCK II cell line expressing the fluorescent protein, but not antigen, or an isotype control antibody was used as negative control for the experiment.

Canine peripheral blood mononuclear cells (PBMCs, Envigo) were used as a source of effector cells. PBMCs were isolated from freshly drawn whole blood, with sodium heparin anticoagulant, using Ficoll-Paque plus (Cytiva, GE17-1440-02) density gradient centrifugation by following the recommended protocol. PBMCs were resuspended in media (PBMC media=RPMI +10% heat inactivated foetal bovine serum+1% penicillin-streptomycin+1% non-essential amino acids+1% L-glutamine+1% sodium pyruvate+2% HEPES) supplemented with 50 ng/ml of recombinant canine IL-2 (R&D systems) and incubated for 24 hours at 37° C. before being used in ADCC assay.

To assess ADCC activity, 10,000 MDCK II cells were co-cultured with PBMCs at an effector: target ratio of 35:1 and a titration of antibodies (11-point 1:3 dilution starting at 10 μg/ml antibody), for 24 h at 37° C., in a 1:1 mix of MDCK II media (DMEM+1% L-glutamine+10% fetal bovine serum) and PBMC media. Rituximab-clGGB was used as the negative (isotype) control antibody.

GFP signal, which is proportional to the number of live cells per well, was used as a measure of the number of live cells remaining in the well at the end of the 24 h incubation. GFP signal was measured on a CLARIOstar (BMG Labtech). Data was analysed using MARS software (BMG Labtech) and percentage of killing in the presence of antibodies was calculated using Microsoft Excel, using wells without antibody as baseline. Graphs were plotted with GraphPad Prism.

FIG. 5 shows ADCC activity was observed in the presence of PMX antibodies. PMX003 antibody shows a stronger ADCC activity than test antibodies 1 E4 and 4E1-7, in both native and afucosylated form. Table 4 summarises the ADCC activity observed for antibodies PMX066 to PMX081.

### Antibody Dependent Cellular Phagocytosis (ADCP) Activity

A suitable method for measuring ADCP activity is described in Ito et al. Leuk Lymphoma. 2015 January; 56(1): 219-225.

### Direct Killing Activity/Apoptosis Assay

Direct killing may be measured as described in Mizuno et al. Scientific Reports, 10, Article 11476 (2020).

### Example 8: Ex Vivo—Whole Blood Assay

To assess the B cell depletion efficiency in ex vivo dog models, 100 μl of freshly drawn dog whole blood was collected with heparin and diluted with 200 μl of RPMI1640+10% heat inactivated fetal bovine serum+1% penicillin/streptomycin/L-glutamine+1% non-essential amino acids+1% sodium pyruvate+2% HEPES buffer. The diluted blood was incubated with or without 10 μg/ml of anti-canine CD20 antibodies for 2 hours, 24 hours and 96 hours at 37° C. After incubation, samples were incubated with Versalyse solution (Beckman Coulter) for 15 minutes at room temperature to lyse red blood cells (RBCs). After washing, the samples were stained with anti-canine CD21 antibody and anti-canine CD8 antibody (both from BioRad), and the number of CD21+ B cells and CD8+ T cells were analysed on CytoFLEX Flow Cytometer (Beckman Coulter). The percentage of B cell depletion was calculated based on the decrease of the ratio of CD21+ B cells to CD8+ T cells as compared to no antibody control. Results in FIG. 6A-B show that PMX003 mAb led to a more efficient killing of B cells in whole canine blood as compared to 1 E4 and 4E1-7 mAbs. Table 4 summarises the ex vivo specific B cell depletion activity observed for antibodies PMX066 to PMX081.

### Example 9: In Vivo Assays

### Tumour Mouse Model

A syngeneic tumour mouse model is generated to evaluate the in vivo tumour killing efficiency of anti-canine CD20 antibodies. The genomic sequences of mouse 129Sv ES cells are modified to replace the mouse CD20 coding region with dog CD20 coding region by homologous recombineering. The targeting vector contains the genomic DNA sequence of dog CD20 coding region flanked by 4-4.5kb of mouse genomic sequences on both sides. After microinjection and germline transmission, the thus-generated mice are bred with Balb/c mice to produce mCD20/dCD20 heterozygous progenies of mixed genetic background.

The mouse lymphoma cell line A20 of Balb/c genetic background is also modified to express dog CD20. The mouse CD20 coding region is replaced by dog CD20 coding sequence by transfecting the A20 cell line with linearized ES cell targeting vector.

To assess the mouse tumour killing efficiency, 1 million dog CD20 knock-in A20 cells (in 100 μl of PBS) are subcutaneously transplanted to mCD20/dCD20 heterozygous mice (6-8 weeks old). Anti-canine CD20-mIGG2a chimeric antibodies (150 μg in 500 μl of PBS) or PBS only are intraperitoneally injected once on Day 0. The size of the tumour is measured every day and the mice are euthanized when the humane endpoints are reached.

### In Vivo Dog Studies

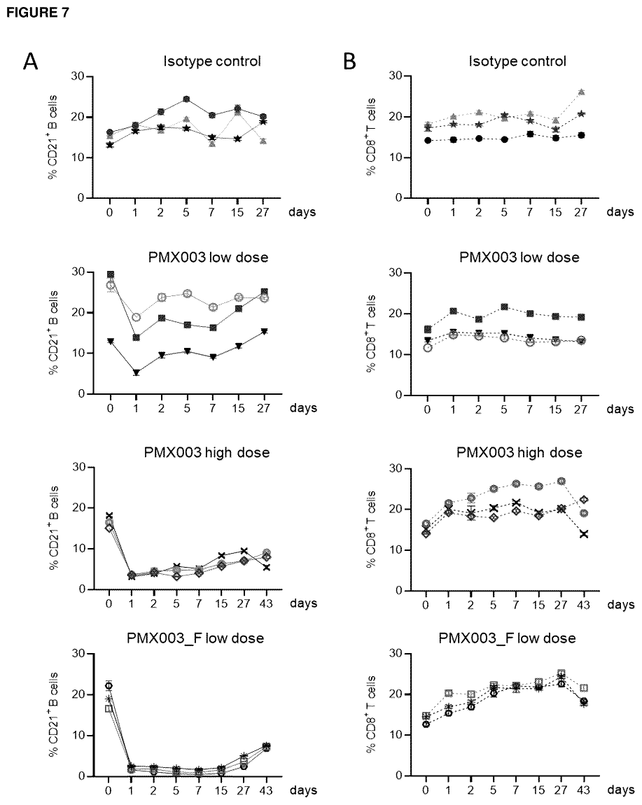

In dog study 1, twelve healthy male beagles (three dogs per test group), 2-3 years old and weighing 8-11 kg, were bred at Avogadro LS, France. All of them were verified to be free of disease or other clinical abnormalities prior to the study. The native format of PMX003 antibody was produced in CHO cells and was administered to two test groups, with one group receiving 0.5 mg/kg antibody and the other receiving 2.5 mg/kg antibody. The a-fucosylated version of PMX003 antibody was produced by expressing it in Fut8 KO CHO cells and it was administered to one test group at 0.5 mg/kg. The control group received isotype control, rituximab-cIGGB, at 2.5 mg/kg. All antibodies were administered on day 0 as a single dose intravenous infusion over 30 minutes. The percentage of CD21+ B cells was monitored and analysed on the following days post antibody administration: day 0 (pre-dose), 1, 2, 5, 7, 15, 27 and 43. 0.5 ml freshly drawn blood with K3EDTA anticoagulant was fixed with 1 ml 1×RBC Lysis/Fixation Solution (BioLegend) for 30 minutes at room temperature in the dark and then diluted with 18 ml of 1× PBS. The fixed and diluted blood samples were kept at +4° C. throughout storage and shipping to the UK. Sample analysis was performed within 48 hours from blood draw. For each sample, the cells were pelleted at 400 g for 5 min, the cell pellets were resuspended in 0.5 ml buffer and 50 μl cells were used for staining of each technical replicate. For each replicate, cells were stained with AF647 conjugated anti-canine CD21 antibody (BioRad, 1:10) and PE conjugated anti-canine CD8 antibody (BioRad, 1:50). Data was acquired using Beckman Coulter CytoFLEX by setting the threshold on DAPI to exclude debris and non-nucleated cells. Percentage of CD21+ B cells and CD8+ T cells in the lymphocyte population was analysed using FlowJo software. B cell depletion was determined by comparing the percentage of CD21+ B cells on the day of assessment post antibody administration to the percentage on day 0 (baseline) for each dog. The percentage of CD8+ T cells was also evaluated and served as the internal control. FIG. 7A-B shows the results of B cell depletion in healthy beagles administered PMX and control antibodies. Depletion was observed when PMX003 was administered in native and a-fucosylated form. The a-fucosylated form was more effective than the native form. PMX003 showed B cell killing at a much lower dose than that observed in a dog study using 1 E4 (Rue et al. Veterinary Immunology and Immunopathology 164(2015), 148-159. In addition, B cell depletion was maintained at a low level for at least 15 days which is longer than observed in a dog study for 4E1-7 (Mizuno et al.).

In dog study 2, fifteen healthy male beagles (three dogs per test group), 2-3 years old and weighing 8-11 kg, were bred at Avogadro LS, France. All of them were verified to be free of disease or other clinical abnormalities prior to the study. The native format of PMX070 and PMX115 antibody was produced in CHO cells and was administered to two test groups per antibody, with one group receiving 0.5 mg/kg antibody and the other receiving 2 mg/kg antibody. The control group received isotype control, rituximab-cIGGB, at 2 mg/kg. All antibodies were administered on day 0 as a single dose intravenous infusion over 30 minutes. The percentage of CD21+ B cells was monitored and analysed on the following days post antibody administration: day 0 (pre-dose), 1, 4, 7, 14, 21 and 28. 1 ml freshly drawn blood with K3EDTA anticoagulant was fixed with 2 ml 1× RBC Lysis/Fixation Solution (BioLegend) for 30 minutes at room temperature in the dark and then diluted with 12 ml of 1× PBS. The fixed and diluted blood samples were kept at +4° C. throughout storage and shipping to the UK. Sample analysis was performed between 48-72 hours from blood draw. For each sample, the cells were pelleted at 400 g for 5 min, the cell pellets were resuspended in 1 ml buffer and 50 μl cells were used for staining of each technical replicate. For each replicate, cells were stained with AF647 conjugated anti-canine CD21 antibody (BioRad, 1:10) and PE conjugated anti-canine CD8 antibody (BioRad, 1:50). Data was acquired using Beckman Coulter CytoFLEX by setting the threshold on DAPI to exclude debris and non-nucleated cells. Percentage of CD21+ B cells and CD8+ T cells in the lymphocyte population was analysed using FlowJo software. B cell depletion was determined by comparing the percentage of CD21+ B cells on the day of assessment post antibody administration to the percentage on day 0 (baseline) for each dog. The percentage of CD8+ T cells was also evaluated and served as the internal control. FIG. 7C shows the results of B cell depletion in healthy beagles administered PMX and control antibodies. Depletion was observed for PMX070 and PMX115 at 0.5 mg/kg and 2 mg/kg doses, and was comparable to the depletion observed with the a-fucosylated form of PMX003.

For pharmacokinetic analysis, the plasma concentration of the mAb drug was monitored and analysed at the following times post antibody administration: Pre-dose, T2 h, T6 h, T24 h, T48 h, T72 h, T120 h, T168 h, T360 h, D27 and D43 in dog study 1 and pre-dose, T2 h, T6 h, T24 h, D4, D7, D14, D21 and D28 in dog study 2. Fresh blood with lithium heparin anticoagulant was placed on ice immediately after blood draw. The blood tubes were centrifuged at 2500 g for 10 min at +5° C. The plasma was collected into polypropylene tubes and kept on ice until storage at −20° C. The plasma was kept frozen till analysis by ELISA. The concentration of mAb is assessed by ELISA using anti-idiotype polyclonal antibodies against PMX mAb as capture antibodies and anti-canine Fc-HRP (Sigma) or anti-canine Fc-biotin (Sigma) plus streptavidin-HRP (BioLegend) as detection antibody. Anti-idiotype polyclonal or monoclonal antibodies against PMX mAb are raised at GenScript by immunisation of rabbits with PMX Fab fragments, hybridoma cell line generation and selection of anti-idiotype antibodies that bind to PMX mAb followed by removal of any cross-reactive antibodies that bind isotype control antibody.

For immunogenicity assessment, the concentration of anti-drug antibodies (ADA) in the frozen plasma is assessed by ELISA after acid dissociation of any preformed complexes between candidate PMX antibodies and anti-drug antibodies. Chimeric versions of candidate antibodies, for example PMX-mouseFc or PMX-humanFc, are generated and a fraction is labelled with biotin. Presence and concentration of anti-drug antibodies is determined using a sandwich ELISA assay format where the unlabelled chimeric antibody is used as a capture reagent and the biotin labelled chimeric antibody along with streptavidin-HRP is used for detection. The anti-idiotype polyclonal antibodies generated for the pharmacokinetic assay above is used as a positive control and to generate a standard curve.

### Example 10: Epitope Mapping

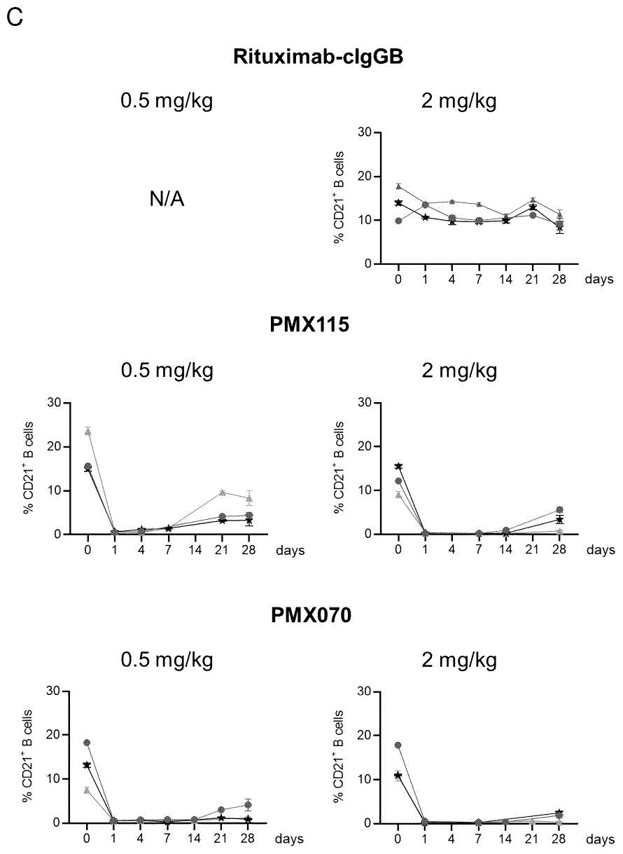

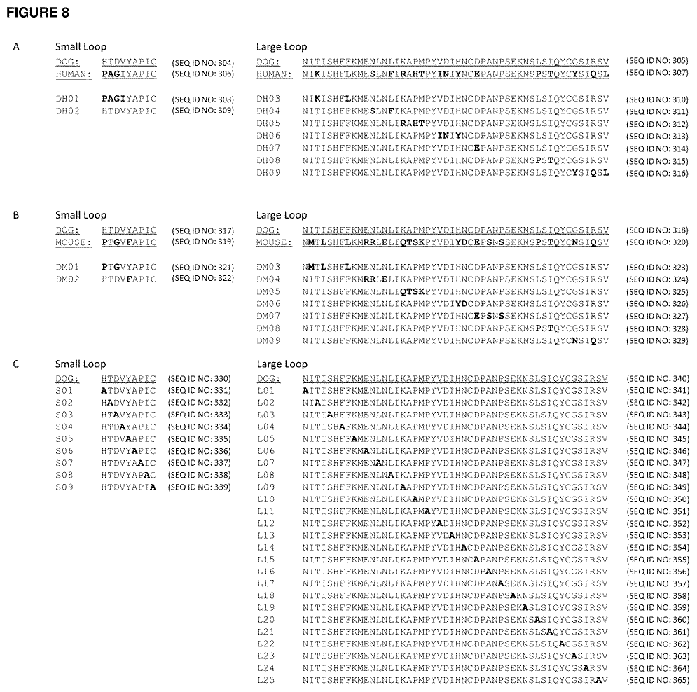

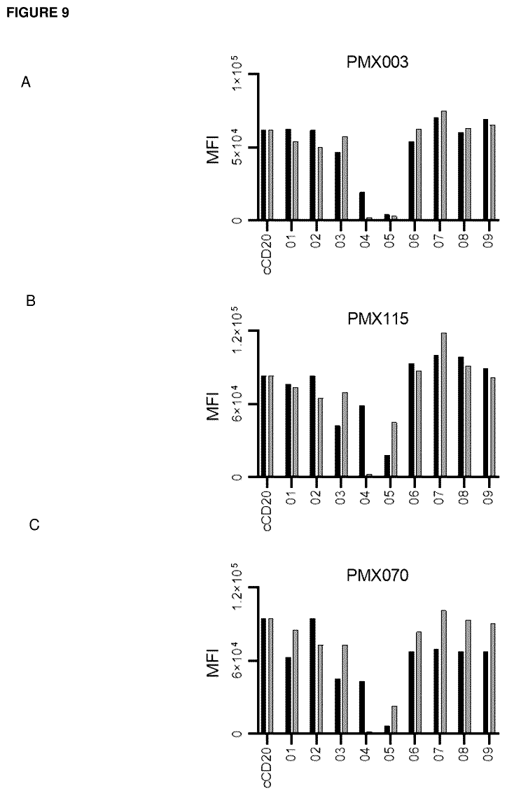

Epitope mapping was conducted to identify the site on canine CD20 antigen that the antibodies specifically bind to. Using site-directed mutagenesis, dog-human chimeric CD20-expressing vectors were generated by dividing the extracellular domain of canine CD20 into 9 regions and replacing each region in the dog sequence with the equivalent location in human sequence (FIG. 8A). The same strategy was used to generate dog-mouse chimeric CD20 (FIG. 8B). Mutated full length cDNA were expressed in MDCK cells as described in Example 2. By comparing the binding of PMX003, PMX115 and PMX070 to these chimeric mutants with the binding to wildtype CD20, the sequence ITISHFFKMENLNLIKAPM (SEQ ID NO. 303) was identified as the epitope of PMX003, PMX115 and PMX070, as the mutation at this position led to a reduced binding in flow cytometric analysis (FIG. 9). “ENLNLIKAPM” (SEQ ID NO. 303, amino acid No. 150-159 in Seq ID NO: 2) was identified as the core epitope within that sequence. This epitope is different from “DIHNCD” which was described as the epitope of the benchmark 1E4 antibody. Another strategy to investigate the epitope is by alanine scanning, in which single alanine mutations are generated at every residue in the small loop and every other residue in the large loop (FIG. 8C).

### Example 11 Developability Criteria

There are several aspects of antibody characterisation including: 1) Transient expression level during small-scale antibody production which correlates to the eventual stable expression level which affects COGS (cost of goods sold). 2) Thermal stability which correlates to a number of properties, including propensity to aggregate, and compatibility with higher concentration formulations. Thermal stability can be reflected by Tm1 value, which is the temperature when 50% of the proteins are unfolded, and Tagg, which is the aggregation temperature. The Tm1 and Tagg values of the lead antibodies were measured using the UNcle protein stability screening platform (Unchained Labs). 3) Size and purity. Fragmentation and aggregation may lead to issues with efficacy or immunogenicity in vivo. The size of the antibodies was determined on non-reduced and reduced SDS-PAGE gel. HPLC-SEC indicated that each candidate product is made up of over 95% monomers. The diameter of the particles was detected using the UNcle machine which allowed us to detect potential issues of aggregation. The developability parameters of PMX003, PMX066, PMX067, PMX069, PMX070, PMX071, PMX072, PMX073, PMX074, PMX075, PMX076, PMX077, PMX078, PMX079, PMX080, PMX081, PMX112, PMX115, PMX122 and PMX124 antibodies were displayed in Table 5.

### Example 12 Analysis of Deglycosylated Antibodies

In addition to the above, a prediction of sequence liabilities was conducted using in silico sequence analysis. An N-linked glycosylation motif (NVT) was identified in the framework region 1 (FR1) of the light chain in PMX003 mAb and also in another 12 mAbs from the lead panel (except PMX070, PMX075 and PMX080). To reduce liabilities by preventing glycosylation at this position the NVT sequon in light chain FR1 of PMX003 was mutated to QVT (PMX006), AVT (PMX007), EVT (PMX008), NVA (PMX009), SVT (PMX010) and TVT (PMX011). De-glycosylation was observed by comparing the size of the light chain of the mutant antibodies with the original PMX003 mAb on a reduced SDS-PAGE gel. Following this, a binding and functional assessment was performed in order to evaluate the impact of these mutations. Flow-cytometry analysis revealed that the binding capacity to canine CD20-expressing cells remained unchanged, apart from QVT and AVT mutants which were slightly reduced (FIG. 3B). Similarly, the killing potency in in vitro ADCC assay (FIGS. 5D and 5E), and ex vivo whole blood assay (FIG. 6C) was not significantly influenced by these mutations. Therefore, it is possible to modify the NVT motif for de-glycosylation without compromising the binding and functional activities.

TABLE 2
SEQUENCES

| SEQ ID | | |
|---|---|---|
| NO: | Description | Sequence |
| 1 | Nucleotide | ATGACAACACCCAGAAATTCAATGAGTGGAACTCTCCCGGTA |
| | sequence of | GATCCTATGAAAAGCCCTACTGCCATGTATCCTGTTCAAAAA |
| | canine CD20 | ATAATTCCCAAAAGGATGCCTTCAGTGGTGGGCCCTACACAA |
| | coding region | AACTTCTTCATGAGGGAATCTAAGACACTGGGGGCTGTCCA |
| | | GATTATGAATGGGCTCTTCCACATTGCCCTAGGCAGCCTCCT |
| | | GATGATTCACACGGATGTCTATGCGCCCATCTGTATAACTAT |
| | | GTGGTACCCTCTCTGGGGAGGCATTATGTTCATCATTTCTGG |
| | | ATCACTCCTGGCAGCAGCGGACAAAAACCCCAGGAAGAGTT |
| | | TGGTCAAAGGAAAAATGATAATGAACTCATTGAGCCTCTTTG |
| | | CTGCTATTTCTGGAATAATTTTTTTGATCATGGACATATTTAAT |
| | | ATTACCATTTCCCATTTTTTTAAAATGGAGAATTTGAATCTTAT |
| | | TAAAGCTCCCATGCCATATGTTGACATACACAACTGTGACCC |
| | | AGCTAACCCCTCTGAGAAAAACTCTTTATCTATACAATATTGT |
| | | GGCAGCATACGATCTGTTTTCTTGGGCGTTTTTGCTGTGATG |
| | | GTGATCTTTACCTTTTTCCAGAAACTTGTGACAGCTGGCATT |
| | | GTTGAGAATGAATGGAAAAAACTGTGCTCTAAACCTAAATCT |
| | | GATGTAGTTGTTCTGTTAGCTGCTGAAGAAAAAAAAGAACAG |
| | | CCGATTGAAACAACAGAAGAAATGGTTGAGCTGACTGAAATA |
| | | GCTTCCCAACCAAAGAAAGAAGAAGACATTGAAATTATTCCA |
| | | GTCCAAGAAGAAGAAGAGGAACTGGAAATAAACTTTGCAGAA |
| | | CCTCCCCAGGAGCAGGAATCTTCACCAATAGAAAACGACAG |
| | | CATCCCTTAA |
| 2 | Amino acid | MTTPRNSMSGTLPVDPMKSPTAMYPVQKIIPKRMPSVVGPTQN |
| | sequence of | FFMRESKTLGAVQIMNGLFHIALGSLLMIHTDVYAPICITMWYPL |
| | canine CD20 | WGGIMFIISGSLLAAADKNPRKSLVKGKMIMNSLSLFAAISGIIFLI |
| | coding region | MDIFNITISHFFKMENLNLIKAPMPYVDIHNCDPANPSEKNSLSIQ |
| | | YCGSIRSVFLGVFAVMVIFTFFQKLVTAGIVENEWKKLCSKPKS |
| | | DVVVLLAAEEKKEQPIETTEEMVELTEIASQPKKEEDIEIIPVQEE |
| | | EEELEINFAEPPQEQESSPIENDSIP |
| 3 | PMX001 heavy | GAGGTGCAGCTGGTGGAGTCTGGGGGAGACCTGGTGAAGC |
| | chain variable | CTGGGGGGTCCCTGAGACTTTCCTGTGTGGCCTCTGGATTC |
| | region | ACCTTCAGTAGCTACCACATGAGCTGGGTCCGCCAGGCTCC |
| | nucleotide | AGGGAAGGGGCTTCAGTGGGTCGCATACATTAATAGTGGTG |
| | sequence | GAAGTAGCACAACCTATGCAGACGCTGTGAAGGGCCGATTC |
| | | ACCATCTCCAGAGACAACGCCAAGAAAACGCTGTATCTTCAG |
| | | ATGAACAGCCTGAGAGCCGAGGACACGGCCGTGTATTACTG |
| | | TTCGACTTTTGACTACTGGGGCCAGGGAACCCTGGTCACCG |
| | | TCTCCTCAG |
| 4 | PMX001 heavy | EVQLVESGGDLVKPGGSLRLSCVASGFTFSSYHMSWVRQAPG |
| | chain variable | KGLQWVAYINSGGSSTTYADAVKGRFTISRDNAKKTLYLQMNS |
| | region amino | LRAEDTAVYYCSTFDYWGQGTLVTVSS |
| | acid sequence | |
| 5 | PMX001 light | TCCTATGTGCTGACACAGCTGCCATCCAGAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATACCAGGAGGCCGACAG |
| | sequence | GGATCCCTGAGCGATTCTCCGGCGCCAACTCGGGGAACACG |
| | | GCCACCCTGACCATCAGCGGGGCCCTGGCCGAGGACGAGG |
| | | CTGACTATTACTGCCAGGTGTGGGACAGCAGTGGTAATGTG |
| | | TTCGGCGGAGGCACCCATCTGACCGTCCTCG |
| 6 | PMX001 light | SYVLTQLPSRNVTLKQPAHITCGGDNIGSKSVHWYQQKLGQAP |
| | chain variable | VLIIYYDTRRPTGIPERFSGANSGNTATLTISGALAEDEADYYCQ |
| | region amino | VWDSSGNVFGGGTHLTVL |
| | acid sequence | |
| 7 | PMX001 heavy | GFTFSSYH (Gly Phe Thr Phe Ser Ser Tyr His) |
| | chain CDR1 | |
| 8 | PMX001 heavy | INSGGSST (Ile Asn Ser Gly Gly Ser Ser Thr) |
| | chain CDR2 | |
| 9 | PMX001 heavy | STFDY (Ser Thr Phe Asp Tyr) |
| | chain CDR3 | |
| 10 | PMX001 light | NIGSKS (Asn Ile Gly Ser Lys Ser) |
| | chain CDR1 | |
| 11 | PMX001 light | YDT (Tyr Asp Thr) |
| | chain CDR2 | |
| 12 | PMX001 light | QVWDSSGNV (Gln Val Trp Asp Ser Ser Gly Asn Val) |
| | chain CDR3 | |
| 13 | PMX002heavy | GAGGTGCAGTTGGTGGAGTCTGGGGGAGACCTGGTGAAGC |
| | chain variable | CTGGGGGGTCCCTGAGACTTTCCTGTGTGGCCTCTGGAATC |
| | region | ATCTTCAGTAACTACCACATGAGCTGGGTCCGCCAGGTTCCA |
| | nucleotide | GGGAAGGGACTTCAGTGGGTCGCATACATTAACAGTGGTGG |
| | sequence | AAGTACTATAAACTATGCAGACGGTGTGAAGGGCCGATTCAC |
| | | CATCTCCAGAGACAACGCCAAGAACTCGCTGTATCTTCAGAT |
| | | GAACAGCCTGAGAGGCGAGGACACGGCCGTGTATTACTGTA |
| | | CGACTTATGACTCCTGGGGCCAGGGAACCCTGGTCACCGTC |
| | | TCCTCAG |
| 14 | PMX002 heavy | EVQLVESGGDLVKPGGSLRLSCVASGIIFSNYHMSWVRQVPGK |
| | chain variable | GLQWVAYINSGGSTINYADGVKGRFTISRDNAKNSLYLQMNSL |
| | region amino | RGEDTAVYYCTTYDSWGQGTLVTVSS |
| | acid sequence | |
| 15 | PMX002 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCAGCAGGCCGACAG |
| | sequence | GGATCCCTGAGCGATTCTCCGGCGCCAACTCGGGGAACACG |
| | | GCCACCCTGACCATCAGCGGGGCCCTGGCCGAGGACGAGG |
| | | CTGACTATTACTGCCAGGTGTGGGACAGCAGTGTTAATGTGT |
| | | TCGGCGGAGGCACCCATCTGACCGTCCTCG |
| 16 | PMX002 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSVNVFGGGTHLTVL |
| | acid sequence | |
| 17 | PMX002 heavy | GIIFSNYH (Gly Ile Ile Phe Ser Asn Tyr His) |
| | chain CDR1 | |
| 18 | PMX002 heavy | INSGGSTI (Ile Asn Ser Gly Gly Ser Thr Ile) |
| | chain CDR2 | |
| 19 | PMX002 heavy | TTYDS (Thr Thr Tyr Asp Ser) |
| | chain CDR3 | |
| 20 | PMX002 light | NIGSKS (Asn Ile Gly Ser Lys Ser) |
| | chain CDR1 | |
| 21 | PMX002 light | YDS (Tyr Asp Ser) |
| | chain CDR2 | |
| 22 | PMX002 light | QVWDSSVNV (Gln Val Trp Asp Ser Ser Val Asn Val) |
| | chain CDR3 | |
| 23 | PMX003 heavy | GAGGTGCAGCTGGTGGAGTCTGGGGGAGACCTGGTGAAGC |
| | chain variable | CTGGGGGGTCCCTGAGACTTTCCTGTGTGGCCTCTGGAATC |
| | region | ACCTTCAGTAGGTACCACATGAGCTGGGTCCGCCAGGCTCC |
| | nucleotide | AGGGAAGGGGCTTCAATGGGTCGCATACATTAACAGTGGTG |
| | sequence | GAAGTAGCACAAGCTATCCAGACGCTGTGAAGGGCCGATTC |
| | | ACCATCTCCAGAGACAACGCCAAGAACACGCTGTATCTTCAG |
| | | ATGGACAGCCTGAGAGCCGAGGACACGGCCCTGTATTACTG |
| | | TACGACCTTTGACTACTGGGGCCAGGGAACCCTGGTCACCG |
| | | TCTCCTCAG |
| 24 | PMX003 heavy | EVQLVESGGDLVKPGGSLRLSCVASGITFSRYHMSWVRQAPG |
| | chain variable | KGLQWVAYINSGGSSTSYPDAVKGRFTISRDNAKNTLYLQMDS |
| | region amino | LRAEDTALYYCTTFDYWGQGTLVTVSS |
| | acid sequence | |
| 25 | PMX003 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAACTGGGCCAGG |
| | nucleotide | CCCCTGTACTGATTATCTATTATGATAGCAGGAGGCCGACAG |
| | sequence | GGATCCCTGAGCGATTCTCCGGCGCCAACTCGGGGAACACG |
| | | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 26 | PMX003 light | SYVLTQLPSKNVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 27 | PMX003 heavy | GITFSRYH (Gly Ile Thr Phe Ser Arg Tyr His) |
| | chain CDR1 | |
| 28 | PMX003 heavy | INSGGSST (Ile Asn Ser Gly Gly Ser Ser Thr) |
| | chain CDR2 | |
| 29 | PMX003 heavy | TTFDY (Thr Thr Phe Asp Tyr) |
| | chain CDR3 | |
| 30 | PMX003 light | NIGSKS (Asn Ile Gly Ser Lys Ser) |
| | chain CDR1 | |
| 31 | PMX003 light | YDS (Tyr Asp Ser) |
| | chain CDR2 | |
| 32 | PMX003 light | QVWDSSANV (Gln Val Trp Asp Ser Ser Ala Asn Val) |
| | chain CDR3 | |
| 33 | PMX004 heavy | GAAGTGCAGCTGGTGGAGTCTGGGGGAGAGCTGGTGAAGC |
| | chain variable | CTGGGGGGTCCCTGAGAGTTTCCTGTGTGGCCTCTGGAATC |
| | region | ACCTTCAGTCGCTACCACATGAGTTGGGTCCGCCAGGCTCC |
| | nucleotide | AGGGAAGGGACTTCAGTGGGTCGCATTCATTAACACTGATA |
| | sequence | GAAGTAGCCCAAAATATGCAGACTCTGTGAAGGGCCGATTC |
| | | ACCATCTCCAGAGACAACGCCAAGAACACGCTGTATCTTCAG |
| | | ATGAACAGCCTGAGAGCCGAGGACACGGCCATTTATTACTG |
| | | TACGAGTTTTGACTACTGGGGCCAGGGAACCCTGGTCACCG |
| | | TCTCCTCAA |
| 34 | PMX004 heavy | EVQLVESGGELVKPGGSLRVSCVASGITFSRYHMSWVRQAPG |
| | chain variable | KGLQWVAFINTDRSSPKYADSVKGRFTISRDNAKNTLYLQMNS |
| | region amino | LRAEDTAIYYCTSFDYWGQGTLVTVSS |
| | acid sequence | |
| 35 | PMX004 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGTAGCAGGCCGACAG |
| | sequence | GGATCCCTGAGCGATTCTCCGGCGCCAACTCGGGGAACACG |
| | | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 36 | PMX004 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSANVFGGGTHLTVL |
| | acid sequence | |
| 37 | PMX004 heavy | GITFSRYH (Gly Ile Thr Phe Ser Arg Tyr His) |
| | chain CDR1 | |
| 38 | PMX004 heavy | INTDRSSP (Ile Asn Thr Asp Arg Ser Ser Pro) |
| | chain CDR2 | |
| 39 | PMX004 heavy | TSFDY (Thr Ser Phe Asp Tyr) |
| | chain CDR3 | |
| 40 | PMX004 light | NIGSKS (Asn Ile Gly Ser Lys Ser) |
| | chain CDR1 | |
| 41 | PMX004 light | YDS (Tyr Asp Ser) |
| | chain CDR2 | |
| 42 | PMX004 light | QVWDSSANV (Gln Val Trp Asp Ser Ser Ala Asn Val) |
| | chain CDR3 | |
| 43 | PMX005 heavy | GAGGTGCAGCTGGTGGAGTCTGGGGGAGACCTGGTGAAGC |
| | chain variable | CTGGGGGGTCCCTGAGACTTTCCTGTGTGGCCTCTGGATTC |
| | region | ACCTTCAGTAGGTACCACATGAGCTGGGTCCGCCAGGCTCC |
| | nucleotide | AGGGAAGGGGCTTCAGTGGGTCGCATACATTAACAGTGGTG |
| | sequence | GAAGTGACTCAGACTATGCAGACGCTGTGAAGGGCCGATTC |
| | | ACCATCTCCAGAGACAACGCCAAGAATACGCTGTATCTTCAG |
| | | ATGAACAGCCTGAGAGCCGAGGACACGGCCGTGTATTATTG |
| | | TACGAGTCTGGACTACTGGGGCCAGGGAACCCTGGTCACCG |
| | | TCTCCTCAG |
| 44 | PMX005 heavy | EVQLVESGGDLVKPGGSLRLSCVASGFTFSRYHMSWVRQAPG |
| | chain variable | KGLQWVAYINSGGSDSDYADAVKGRFTISRDNAKNTLYLQMNS |
| | region amino | LRAEDTAVYYCTSLDYWGQGTLVTVSS |
| | acid sequence | |
| 45 | PMX005 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCAGCAGGCCGACAG |
| | sequence | GGATCCCTGAGCGATTCTCCGGCGCCAACTCGGGGAACACG |
| | | GCCACCCTGACCATCAGCGGGGCCCTGGCCGAGGACGAGG |
| | | CTGACTATTACTGTCAGGTGTGGGACAGGAGTGGTAATGTGT |
| | | TCGGCGGAGGCACCCATCTGACCGTCCTCG |
| 46 | PMX005 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDRSGNVFGGGTHLTVL |
| | acid sequence | |
| 47 | PMX005 heavy | GFTFSRYH (Gly Phe Thr Phe Ser Arg Tyr His) |
| | chain CDR1 | |
| 48 | PMX005 heavy | INSGGSDS (Ile Asn Ser Gly Gly Ser Asp Ser) |
| | chain CDR2 | |
| 49 | PMX005 heavy | TSLDY (Thr Ser Leu Asp Tyr) |
| | chain CDR3 | |
| 50 | PMX005 light | NIGSKS (Asn Ile Gly Ser Lys Ser) |
| | chain CDR1 | |
| 51 | PMX005 light | YDS (Tyr Asp Ser) |
| | chain CDR2 | |
| 52 | PMX005 light | QVWDRSGNV (Gln Val Trp Asp Arg Ser Gly Asn Val) |
| | chain CDR3 | |
| 53 | PMX006light | SYVLTQLPSKQVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 54 | PMX007light | SYVLTQLPSKAVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 55 | PMX008 light | SYVLTQLPSKEVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 56 | PMX009 light | SYVLTQLPSKNVALKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 57 | PMX010 light | SYVLTQLPSKSVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 58 | PMX011 light | SYVLTQLPSKTVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 59 | PMX003light | SYVLTQLPSKNVTLKQPAHITCGGD (Ser Tyr Val Leu Thr Gln |
| | chain FR1 | Leu Pro Ser Lys Asn Val Thr Leu Lys Gln Pro Ala His Ile Thr Cys |
| | | Gly Gly Asp) |
| 60 | PMX006light | SYVLTQLPSKQVTLKQPAHITCGGD (Ser Tyr Val Leu Thr Gln |
| | chain FR1 | Leu Pro Ser Lys Gln Val Thr Leu Lys Gln Pro Ala His Ile Thr Cys |
| | | Gly Gly Asp) |
| 61 | PMX007 light | SYVLTQLPSKAVTLKQPAHITCGGD (Ser Tyr Val Leu Thr Gln |
| | chain FR1 | Leu Pro Ser Lys Ala Val Thr Leu Lys Gln Pro Ala His Ile Thr Cys |
| | | Gly Gly Asp) |
| 62 | PMX008 light | SYVLTQLPSKEVTLKQPAHITCGGD (Ser Tyr Val Leu Thr Gln |
| | chain FR1 | Leu Pro Ser Lys Glu Val Thr Leu Lys Gln Pro Ala His Ile Thr Cys |
| | | Gly Gly Asp) |
| 63 | PMX009 light | SYVLTQLPSKNVALKQPAHITCGGD (Ser Tyr Val Leu Thr Gln |
| | chain FR1 | Leu Pro Ser Lys Asn Val Ala Leu Lys Gln Pro Ala His Ile Thr Cys |
| | | Gly Gly Asp) |
| 64 | PMX010 light | SYVLTQLPSKSVTLKQPAHITCGGD (Ser Tyr Val Leu Thr Gln |
| | chain FR1 | Leu Pro Ser Lys Ser Val Thr Leu Lys Gln Pro Ala His Ile Thr Cys |
| | | Gly Gly Asp) |
| 65 | PMX011 light | SYVLTQLPSKTVTLKQPAHITCGGD (Ser Tyr Val Leu Thr Gln |
| | chain FR1 | Leu Pro Ser Lys Thr Val Thr Leu Lys Gln Pro Ala His Ile Thr Cys |
| | | Gly Gly Asp) |
| 66 | Canine IGGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | amino acid | HPASKTKVDKPVPKRENGRVPRPPDCPKCPAPEMLGGPSVFIF |
| | sequence | PPKPKDTLLIARTPEVTCVVVDLDPEDPEVQISWFVDGKQMQTA |
| | | KTQPREEQFNGTYRVVSVLPIGHQDWLKGKQFTCKVNNKALPS |
| | | PIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 67 | Canine IGLC5 | GQPKASPSVTLFPPSSEELGANKATLVCLISDFYPSGVTVAWKA |
| | constant region | DGSPITQGVETTKPSKQSNNKYAASSYLSLTPDKWKSHSSFSC |
| | amino acid | LVTHEGSTVEKKVAPAECS |
| | sequence | |
| 68 | Codon- | GAGGTGCAGCTGGTGGAGAGCGGCGGCGACCTGGTGAAGC |
| | optimised | CTGGCGGCAGCCTGAGACTGAGCTGCGTGGCAAGCGGCAT |
| | nucleotide | CACCTTCAGCAGATACCACATGAGCTGGGTGAGACAAGCCC |
| | sequence of | CTGGCAAGGGCCTGCAGTGGGTGGCCTACATCAACAGCGG |
| | PMX003 heavy | CGGCAGCAGCACAAGCTACCCTGACGCCGTGAAGGGCAGA |
| | chain variable | TTCACCATCAGCAGAGACAACGCCAAGAACACCCTGTACCT |
| | region | GCAGATGGACAGCCTGAGAGCCGAGGACACCGCCCTGTACT |
| | | ACTGCACCACCTTCGACTACTGGGGCCAAGGCACCCTGGTG |
| | | ACCGTGAGCAGCG |
| 69 | Codon- | AGCTACGTGCTGACACAGCTGCCTAGCAAGAACGTGACCCT |
| | optimised | GAAGCAGCCTGCCCACATCACCTGCGGCGGCGACAACATCG |
| | nucleotide | GCAGCAAGAGCGTGCACTGGTATCAGCAGAAGCTGGGCCAA |
| | sequence of | GCCCCTGTGCTGATCATCTACTACGACAGCAGAAGACCTAC |
| | PMX003 light | CGGCATCCCTGAGAGATTCAGCGGCGCCAACAGCGGCAACA |
| | chain variable | CCGCCACCCTGACCATCAGCGGCGCCCTGGCCGAGGACGA |
| | region | GGCCGACTACTACTGCCAAGTGTGGGACAGCAGCGCCAACG |
| | | TGTTCGGCGGCGGCACCCACCTGACCGTGCTGG |
| 70 | Campath leader | MGWSCIILFLVATATGVHS |
| | sequence | |
| 71 | Native leader of | MESALSWVFLVTILKGVQG |
| | PMX001, | |
| | PMX002, | |
| | PMX003, | |
| | PMX004 and | |
| | PMX005 heavy | |
| | chain | |
| 72 | Native leader of | MAWTHLLLSLLALCTGSVA |
| | PMX001, | |
| | PMX002, | |
| | PMX003, | |
| | PMX004and | |
| | PMX005 light | |
| | chain | |
| 73 | Canine IGK | RNDAQPAVYLFQPSPDQLHTGSASVVCLLNSFYPKDINVKWKV |
| | constant region | DGVIQDTGIQESVTEQDKDSTYSLSSTLTMSSTEYLSHELYSCEI |
| | amino acid | THKSLPSTLIKSFQRSECQRVD |
| | sequence | |
| 74 | cIGHV3-5 IMGT | EVQLVESGGDLVKPGGSLRLSCVASGFTFSSYHMSWVRQAPG |
| | reference | KGLQWVAYINSGGSSTSYADAVKGRFTISRDNAKNTLYLQMNS |
| | sequence | LRAEDTAVYYCAS |
| 75 | CIGHJ4 IMGT | NFDYWGQGTLVTVSS |
| | reference | |
| | sequence | |
| 76 | CIGLV3-3 IMGT | SYVLTQLPSKNVTLKQPAHITCGGDNIGSKSVHWYQQKLGQAP |
| | reference | VLIIYYDSSRPTGIPERFSGANSGNTATLTISGALAEDEADYYCQ |
| | sequence | VWDSSAKA |
| 77 | CIGLJ3 IMGT | SVFGGGTHLTVL |
| | reference | |
| | sequence | |
| 78 | PMX066 heavy | ATGGAGTCTGCGTTCAGCTGGGTGTTCCTTGTCACTATTTTA |
| | chain variable | AAAGGTGTCCAGGGTGCGGTGCAGCTGGTGGAGTCAGGGG |
| | region | GAGACCTGGTGAAGCCTGGGGGGTCCCTGAGACTTTCCTGT |
| | nucleotide | GTGGCCTCTGGAATCACCTTCAGTAGGTACCACATGAGTTG |
| | sequence | GGTCCGCCAGGCTCCAGGGAAGGGGCTTCAGTGGGTCGCA |
| | | TACATTAATAGTGGTGGAAGTGCCACAAGTTATACAGACGCT |
| | | GTGAAGGGCCGATTCAGCATCTCCAGAGACAACGGCAAAAA |
| | | CACGCTTTATCTTCAGATGAATAGCCTGAGAGTCGAGGACTC |
| | | GGCCGTCTATTACTGTACGACCCTTGACCTCTGGGGCCAGG |
| | | GAACCCTGGTCACCGTCTCCTCAG |
| 79 | PMX066 heavy | AVQLVESGGDLVKPGGSLRLSCVASGITFSRYHMSWVRQAPG |
| | chain variable | KGLQWVAYINSGGSATSYTDAVKGRFSISRDNGKNTLYLQMNS |
| | region amino | LRVEDSAVYYCTTLDLWGQGTLVTVSS |
| | acid sequence | |
| 80 | PMX066 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGA |
| | | TAGCCGCAGGCCGACAGGGATCCCTGAGCGATTCTCCGGC |
| | | GCCAACTCGGGGAACACGGCCACCCTGACCATCAACGGGG |
| | | CCCTGGCCGAGGACGAGGCTGACTATTACTGTCAGATGTGG |
| | | GACAGCAGTGCTAATGTGTTCGGCGGAGGCACCCATCTGAC |
| | | CGTCCTCG |
| 81 | PMX066 light | SYVLTQLPSKNVTLKQPAHITCGGDNIGSKSVHWYQQKLGQAP |
| | chain variable | VLIIYYDSRRPTGIPERFSGANSGNTATLTINGALAEDEADYYCQ |
| | region amino | MWDSSANVFGGGTHLTVL |
| | acid sequence | |
| 82 | PMX066 heavy | GITFSRYH |
| | chain CDR1 | |
| 83 | PMX066 heavy | INSGGSAT |
| | chain CDR2 | |
| 84 | PMX066 heavy | TTLDL |
| | chain CDR3 | |
| 85 | PMX066 light | NIGSKS |
| | chain CDR1 | |
| 86 | PMX066 light | YDS |
| | chain CDR2 | |
| 87 | PMX066 light | QMWDSSANV |
| | chain CDR3 | |
| 88 | PMX067 heavy | ATGGAGTCTGCGCTCAGCTGGGTGTTCCTTGTCACTATTTTA |
| | chain variable | AAAGGTGTCCAGGGTGAGATACAACTGGTGGAGTCTGGGGG |
| | region | AGACCTGGTGAAGCCTGGGGGGTCCCTGAGACTTTCCTGTG |
| | nucleotide | TGGCCTCTGGATTCACCTTCAGTCGCTACCACATGAGCTGG |
| | sequence | GTCCGCCAGGCTCCAGGGAAGGGGCTTCAGTGGGTCGCAT |
| | | ATATTAACACTGGTGGAAGTAACACAAACTATGCAGACGCTG |
| | | TGAAGGGCCGATTCACCATCTCCAGAGACAACGCCAAGAAC |
| | | ACGCTGTATCTTCAGATGAACAGCCTGAGAGCCGAGGACAC |
| | | GGCCGTGTATTACTGTACGACCCTTGACTCCTGGGGCCAGG |
| | | GAACCCTGGTCACCGTCTCCTCAG |
| 89 | PMX067 heavy | EIQLVESGGDLVKPGGSLRLSCVASGFTFSRYHMSWVRQAPG |
| | chain variable | KGLQWVAYINTGGSNTNYADAVKGRFTISRDNAKNTLYLQMNS |
| | region amino | LRAEDTAVYYCTTLDSWGQGTLVTVSS |
| | acid sequence | |
| 90 | PMX067 light | ATGGCCTGGACCCACCTCCTCCTGAGCCTCCTGGCTCTCTG |
| | chain variable | CACAGGTTCTGTGGCCTCCTATGTGCTGACACAGCTGCCATT |
| | region | CAAAAATGTGACCCTGAAGCAGCCGGCCCACATCACCTGTG |
| | nucleotide | GGGGAGACAACATTGGAAGTAAAAGTGTTCACTGGTACCAG |
| | sequence | CAGAAGCTGGGCCAGGCCCCTGTATTGATTATCTATTATGAT |
| | | AGTAGAAGGCCGACAGGGATCCCTGAGCGATTCTCCGGCGC |
| | | CAACTCGGGGAACACGGCCACCCTGACCATCAGCGGGGCC |
| | | CTGGCCGAGGACGAGGCTGACTATTACTGCCAGGTGTGGGA |
| | | CAGTAGTGTTAATGTGTTCGGCGGAGGCACCCATCTGACCG |
| | | TCCTCG |
| 91 | PMX067 light | SYVLTQLPFKNVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSVNVFGGGTHLTVL |
| | acid sequence | |
| 92 | PMX067 heavy | GFTFSRYH |
| | chain CDR1 | |
| 93 | PMX067 heavy | INTGGSNT |
| | chain CDR2 | |
| 94 | PMX067 heavy | TTLDS |
| | chain CDR3 | |
| 95 | PMX067 light | NIGSKS |
| | chain CDR1 | |
| 96 | PMX067 light | YDS |
| | chain CDR2 | |
| 97 | PMX067 light | QVWDSSVNV |
| | chain CDR3 | |
| 98 | PMX068 heavy | ATGGAGTCTGCGCTCAGCTGGGTGTTCCTTGTCACTATTTTA |
| | chain variable | AAAGGTGTCCAGGGTGAACTACAGCTGGTGGAGTCTGGGGG |
| | region | AGACCTGGTGAAGCCTGGGGGGTCCCTGAGACTTTCCTGTG |
| | nucleotide | TGGCCTCCGGATTCACCTTCAGTAGATACTACTTGAACTGGG |
| | sequence | TCCGCCAGGCTCCAGGGAAGGGGCTTCAGTGGGTCGCATTC |
| | | ATTACCAGTGGTGGCAGTAGCACAAACTATGCAGACGCTGT |
| | | GAAGGGCCGATTCACCATCTCCAGAGACAACGCCAAGAACA |
| | | CACTGTATCTTCAGATGAATAGCCTGAGAGCCGAGGACACG |
| | | GCCGTTTATTACTGTACGAGCTTCGACTCCTGGGGCCAGGG |
| | | AACCCTGGTCACCGTCTCCTCAG |
| 99 | PMX068 heavy | ELQLVESGGDLVKPGGSLRLSCVASGFTFSRYYLNWVRQAPG |
| | chain variable | KGLQWVAFITSGGSSTNYADAVKGRFTISRDNAKNTLYLQMNS |
| | region amino | LRAEDTAVYYCTSFDSWGQGTLVTVSS |
| | acid sequence | |
| 100 | PMX068 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTAATTATCTATTATGA |
| | | TAGCAGCAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACAGCAGTGCTAATGTGTTCGGCGGAGGCACCCATCTGAC |
| | | CGTCCTCG |
| 101 | PMX068 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSANVFGGGTHLTVL |
| | acid sequence | |
| 102 | PMX068 heavy | GFTFSRYY |
| | chain CDR1 | |
| 103 | PMX068 heavy | ITSGGSST |
| | chain CDR2 | |
| 104 | PMX068 heavy | TSFDS |
| | chain CDR3 | |
| 105 | PMX068 light | NIGSKS |
| | chain CDR1 | |
| 106 | PMX068 light | YDS |
| | chain CDR2 | |
| 107 | PMX068 light | QVWDSSANV |
| | chain CDR3 | |
| 108 | PMX069 heavy | ATGGAGTCTGCGCTCAGCTGGGTGTTCCTTGTCACTATTTTA |
| | chain variable | AAAGGTGTCCAGGGTGAGATGCAGCTGGTGGAATCTGGGG |
| | region | GAGCCCTGGTGAAGCCTGGGGGGTCCCTGAGACTCTCCTGT |
| | nucleotide | GTGGCCTCTGGATTCACCTTTAGGAGATACCACATGAGTTGG |
| | sequence | GTCCGCCAGGCTCCAGGACAGGGGCTTCAGTGGGTCGCAT |
| | | ACATTGACAGTGATGGAAGTCCTACAAGTTATGGAGACTCTG |
| | | TGAAGGGCCGATTCACCATCTCCAGAGACAACGCCAAGAAT |
| | | ACGCTATTTCTTCAGATGAACAGTCTGAGAGTCGACGACACG |
| | | GCCGTATATTACTGTACGACTCTTGACTACTGGGGCCGGGG |
| | | AACCCTGGTCACCGTCTCCTCAG |
| 109 | PMX069 heavy | EMQLVESGGALVKPGGSLRLSCVASGFTFRRYHMSWVRQAPG |
| | chain variable | QGLQWVAYIDSDGSPTSYGDSVKGRFTISRDNAKNTLFLQMNS |
| | region amino | LRVDDTAVYYCTTLDYWGRGTLVTVSS |
| | acid sequence | |
| 110 | PMX069 light | ATGGCCTGGACCCACCTCCTCCTGAGCCTCCTGGCTCTCTG |
| | chain variable | CACAGGTTCTGTGGCCTCCTATGTGCTGACACAGGTGCCAT |
| | region | CCAAAAATGTGACCCTGAAGCAGCCGGCCCACATCACCTGT |
| | nucleotide | GGGGGAGACAACATTGGAGGTAAAAGTGTTCACTGGTACCA |
| | sequence | GCAGAAGCTGGGCCAGGCCCCTCTACTGATTATCTATTATGA |
| | | TACCAGGAGGCCGACAGGGATCCCTGAGCGCTTCTCCGGC |
| | | GCCAACTCGGGGAACACGGCCACCCTGACCATCAGCAGGG |
| | | CCCTGGCCGAGGACGAGGCTGACTATTACTGCCAGGTGTGG |
| | | GACAGCAGTGGTAATGTGTTCGGCGGAGGCACCCATCTGAC |
| | | CGTCATCG |
| 111 | PMX069 light | SYVLTQVPSKNVTLKQPAHITCGGDNIGGKSVHWYQQKLGQAP |
| | chain variable | LLIIYYDTRRPTGIPERFSGANSGNTATLTISRALAEDEADYYCQ |
| | region amino | VWDSSGNVFGGGTHLTVI |
| | acid sequence | |
| 112 | PMX069 heavy | GFTFRRYH |
| | chain CDR1 | |
| 113 | PMX069 heavy | IDSDGSPT |
| | chain CDR2 | |
| 114 | PMX069 heavy | TTLDY |
| | chain CDR3 | |
| 115 | PMX069 light | NIGGKS |
| | chain CDR1 | |
| 116 | PMX069 light | YDT |
| | chain CDR2 | |
| 117 | PMX069 light | QVWDSSGNV |
| | chain CDR3 | |
| 118 | PMX070 heavy | ATGGAGTCTGCGCTCAGCTGGGTGTTCCTTGTCACTATTTTA |
| | chain variable | AAAGGTGTCCAGGGTGAGATGCAACTGGTGGAGTCTGGGG |
| | region | GAGACCTGGTGAAGCCTGGGGGGTCCCTGAGACTTTCCTGT |
| | nucleotide | GTGGCCTCTGGATTCACCTTCAGTCGCTACCACATGAGCTG |
| | sequence | GGTCCGCCAGGCTCCAGGGAAGGGGCTTCAGTGGGTCGCA |
| | | TACATTAATAGTGGTGGAAGTCCCACAAACTATGCAGACGCT |
| | | GTGAAGGGCCGATTCACCATCTCCAGAGACAACGCCAAGAA |
| | | CACACTATATCTTCAGATGAACAGCCTGAGAGCCGAGGACA |
| | | CGGCCGTGTATTACTGTACGACCTACGACCAGTGGGGCCAG |
| | | GGAACCCTGGTCACCGTCTCCTCAG |
| 119 | PMX070 heavy | EMQLVESGGDLVKPGGSLRLSCVASGFTFSRYHMSWVRQAPG |
| | chain variable | KGLQWVAYINSGGSPTNYADAVKGRFTISRDNAKNTLYLQMNS |
| | region amino | LRAEDTAVYYCTTYDQWGQGTLVTVSS |
| | acid sequence | |
| 120 | PMX070 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAGTGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGA |
| | | TAGCAGCAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACACCAGTGCTAATGTGTTCGGCGGAGGCACCCATCTGAC |
| | | CGTCCTCG |
| 121 | PMX070 light | SYVLTQLPSKSVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDTSANVFGGGTHLTVL |
| | acid sequence | |
| 122 | PMX070 heavy | GFTFSRYH |
| | chain CDR1 | |
| 123 | PMX070 heavy | INSGGSPT |
| | chain CDR2 | |
| 124 | PMX070 heavy | TTYDQ |
| | chain CDR3 | |
| 125 | PMX070 light | NIGSKS |
| | chain CDR1 | |
| 126 | PMX070 light | YDS |
| | chain CDR2 | |
| 127 | PMX070 light | QVWDTSANV |
| | chain CDR3 | |
| 128 | PMX071 heavy | ATGGAGTCTGCGCTCAGCTGGGTGTTCCTTGTCACTATTTTA |
| | chain variable | AAAGGTGTCCAGGGTGAGGTGCACCTGGTGGAGTCTGGGG |
| | region | GAGACCTGGTGAAGCCTGGGGGGTCCCTGAGACTTTCCTGT |
| | nucleotide | GTGGCCTCTGGAATCACCTTCAATAGATATTATATGAACTGG |
| | sequence | GTCCGCCAGACTCCAGGGAAGGGGCTTCAGTGGGTCGCATA |
| | | CATAGACAGTGGTGGAAGTCCCACAACCTATGCAGACGCTG |
| | | TAAAGGGCCGATTCACCATCTCCAGAGACAACGCCAAGAAC |
| | | ACGTTGTATCTTCAGATGAACAGCCTGAGAGCCGAGGACAC |
| | | GGCCGTATATTACTGTACGAGTTTTGACTACTGGGGCCAGG |
| | | GAACCCTGGTCACCGTCTCCTCAG |
| 129 | PMX071 heavy | EVHLVESGGDLVKPGGSLRLSCVASGITFNRYYMNWVRQTPG |
| | chain variable | KGLQWVAYIDSGGSPTTYADAVKGRFTISRDNAKNTLYLQMNS |
| | region amino | LRAEDTAVYYCTSFDYWGQGTLVTVSS |
| | acid sequence | |
| 130 | PMX071 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTATACTGATTATCTATTATGA |
| | | TACCAGCAGGCCGACAGGGATCCCTGAGCGATTCTCCGGCG |
| | | CCAACTCGGGGAACACGGCCACCCTGACCATCAGCGGGGC |
| | | CCTGGCCGAGGACGAGGCTGACTATTACTGCCAGGTGTGGG |
| | | ACAGCAGTGCTAATGTGTTCGGCGGAGGCACCCATCTGACC |
| | | GTCCTCG |
| 131 | PMX071 light | SYVLTQLPSKNVTLKQPAHITCGGDNIGSKSVHWYQQKLGQAPI |
| | chain variable | LIIYYDTSRPTGIPERFSGANSGNTATLTISGALAEDEADYYCQV |
| | region amino | WDSSANVFGGGTHLTVL |
| | acid sequence | |
| 132 | PMX071 heavy | GITFNRYY |
| | chain CDR1 | |
| 133 | PMX071 heavy | IDSGGSPT |
| | chain CDR2 | |
| 134 | PMX071 heavy | TSFDY |
| | chain CDR3 | |
| 135 | PMX071 light | NIGSKS |
| | chain CDR1 | |
| 136 | PMX071 light | YDT |
| | chain CDR2 | |
| 137 | PMX071 light | QVWDSSANV |
| | chain CDR3 | |
| 138 | PMX072 heavy | ATGGAGTCTGCGCTCAGCTGGGTGTTCCTTGTCACTATTTTA |
| | chain variable | AAAGGTGTCCAGGGTGAGGTGCAGCTGGTGGAGTCTGGGG |
| | region | GAGACCTGGTGAAGCCTGGGGGGTCCCTGAGACTTTCCTGT |
| | nucleotide | GTGGCCTCTGGATTCACCTTCAGTAGGTACCACATGAGCTG |
| | sequence | GGTCCGCCAGGCTCCAGGGAAGGGGCTTCAGTGGGTCGCA |
| | | TTCATTAACACTGGTAGAAGTAGCACAAACTATGCAGACGCT |
| | | GTGGCGGGCCGATTCACCATCTCCAGAGACAACGCCAAGAA |
| | | CACGCTGTATCTTCAGATGAACAGCCTGAGAGCCGAGGACA |
| | | CGGCCGTGTATTACTGTTCGACCCTCGACTACTGGGGCCAG |
| | | GGAACCCTGGTCACCGTCTCCTCAG |
| 139 | PMX072 heavy | EVQLVESGGDLVKPGGSLRLSCVASGFTFSRYHMSWVRQAPG |
| | chain variable | KGLQWVAFINTGRSSTNYADAVAGRFTISRDNAKNTLYLQMNS |
| | region amino | LRAEDTAVYYCSTLDYWGQGTLVTVSS |
| | acid sequence | |
| 140 | PMX072 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGA |
| | | TAGCAGCAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACAGCAGTGCTAATGTGTTCGGCGGAGGCACCCATCTGAC |
| | | CGTCCTCG |
| 141 | PMX072 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSANVFGGGTHLTVL |
| | acid sequence | |
| 142 | PMX072 heavy | GFTFSRYH |
| | chain CDR1 | |
| 143 | PMX072 heavy | INTGRSST |
| | chain CDR2 | |
| 144 | PMX072 heavy | STLDY |
| | chain CDR3 | |
| 145 | PMX072 light | NIGSKS |
| | chain CDR1 | |
| 146 | PMX072 light | YDS |
| | chain CDR2 | |
| 147 | PMX072 light | QVWDSSANV |
| | chain CDR3 | |
| 148 | PMX073 heavy | ATGGAGTCTGCGCTCAGCTGGGTGTTCCTTGTCACTATTTTA |
| | chain variable | AAAGGTGTCCAGGGTGAAGTACAGCTGGTGGAGTCTGGGG |
| | region | GAGACCTGGTGAAGCCTGGGGGGTCCCTGAGACTTTCCTGT |
| | nucleotide | GTGGCCTCCGGATTCACCTTCAGTAGATACTACATGAACTGG |
| | sequence | GTCCGCCAGGCTCCAGGGAAGGGGCTTCAGTGGGTCGCAT |
| | | TCATTACCAGTGGTGGAAGTAGCACAAGCTATGGAGACGCT |
| | | GTGAAGGGCCGATTCACCATCTCCAGAGACAACGCCAAGAA |
| | | CACGCTGTATCTTCAGATGAACAGCCTGAGAGCCGAGGACA |
| | | CGGCCGTGTATTACTGTACGAGCTTCGACTACTGGGGCCAG |
| | | GGAACCCTGGTCACCGTCTCCTCAG |
| 149 | PMX073 heavy | EVQLVESGGDLVKPGGSLRLSCVASGFTFSRYYMNWVRQAPG |
| | chain variable | KGLQWVAFITSGGSSTSYGDAVKGRFTISRDNAKNTLYLQMNS |
| | region amino | LRAEDTAVYYCTSFDYWGQGTLVTVSS |
| | acid sequence | |
| 150 | PMX073 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGC |
| | | TAGCAGCAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACAGCAGTGCTAATGTGTTCGGCGGAGGCACCCATCTGAC |
| | | CGTCCTCG |
| 151 | PMX073 light | SYVLTQLPSKNVTLKQPAHITCGGDNIGSKSVHWYQQKLGQAP |
| | chain variable | VLIIYYASSRPTGIPERFSGANSGNTATLTISGALAEDEADYYCQ |
| | region amino | VWDSSANVFGGGTHLTVL |
| | acid sequence | |
| 152 | PMX073 heavy | GFTFSRYY |
| | chain CDR1 | |
| 153 | PMX073 heavy | ITSGGSST |
| | chain CDR2 | |
| 154 | PMX073 heavy | TSFDY |
| | chain CDR3 | |
| 155 | PMX073 light | NIGSKS |
| | chain CDR1 | |
| 156 | PMX073 light | YAS |
| | chain CDR2 | |
| 157 | PMX073 light | QVWDSSANV |
| | chain CDR3 | |
| 158 | PMX074 heavy | ATGGAGTCTGCGCTCAGCTGGGTGTTCCTTGTCATTATTTTA |
| | chain variable | AAAGGTGTCCAGGGCGAGGTGCAGTTGGTGGAGTCTGGGG |
| | region | GAGACCTGGTGAAGCCTGGGGGGTCCCTGAGACTTTCCTGT |
| | nucleotide | GTGGCCTCTGGATTCACCTTCAGTAGGTATTACATGACCTGG |
| | sequence | GTCCGCCAGGCTCCAGGGAAGGGGCTTCAGTGGGTCGCAT |
| | | ACATTAACAGTGATAGAAGAAGCACAACCTATGCAGACGCTG |
| | | TGAAGGGCCGATTCACCATCTCCAGAGACAACGCCAAAAAC |
| | | ACGCTGTATCTTCAGATGAACAGCCTGAGAGCCGAGGACAC |
| | | GGCCGTATATTACTGTACGACCCTTGAATATTGGGGCCAGG |
| | | GAACCCTGGTCACCGTCTCCTCAG |
| 159 | PMX074 heavy | EVQLVESGGDLVKPGGSLRLSCVASGFTFSRYYMTWVRQAPG |
| | chain variable | KGLQWVAYINSDRRSTTYADAVKGRFTISRDNAKNTLYLQMNS |
| | region amino | LRAEDTAVYYCTTLEYWGQGTLVTVSS |
| | acid sequence | |
| 160 | PMX074 light | ATGGCCTGGACCCACCTCCTCCTGAGCCTCCTGGCTCTCTG |
| | chain variable | CACAGGCTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGA |
| | | TAGCCGCAGGCCGACAGGGATCCCTGAGCGATTCTCCGGC |
| | | GCCAACTCGGGGAACACGGCCACCCTGACCATCAACGGGG |
| | | CCCTGGCCGAGGACGAGGCTGACTATTACTGCCAGGTGTGG |
| | | GACAGCAGTGCTAATGTGTTCGGCGGAGGCACCCATCTGAC |
| | | CGTCCTCG |
| 161 | PMX074 light | SYVLTQLPSKNVTLKQPAHITCGGDNIGSKSVHWYQQKLGQAP |
| | chain variable | VLIIYYDSRRPTGIPERFSGANSGNTATLTINGALAEDEADYYCQ |
| | region amino | VWDSSANVFGGGTHLTVL |
| | acid sequence | |
| 162 | PMX074 heavy | GFTFSRYY |
| | chain CDR1 | |
| 163 | PMX074 heavy | INSDRRST |
| | chain CDR2 | |
| 164 | PMX074 heavy | TTLEY |
| | chain CDR3 | |
| 165 | PMX074 light | NIGSKS |
| | chain CDR1 | |
| 166 | PMX074 light | YDS |
| | chain CDR2 | |
| 167 | PMX074 light | QVWDSSANV |
| | chain CDR3 | |
| 168 | PMX075 heavy | ATGGAGTCTGCGCTCAGCTGGGTGTTCCTTGTCACTATTTTA |
| | chain variable | AAAGGTGTCCAGGGTGAGGTGCAGTTGGTGGAGTCTGGGG |
| | region | GAGACCTGGTGAAGCCGGGGGGGTCCCTGAGACTTTCCTGT |
| | nucleotide | GTGGCCTCCGGAATCACCTTCAGTAGATATCACATGAGCTG |
| | sequence | GGTCCGCCAGGCTCCGGGGAAGGGGCTTCAGTGGGTCGCA |
| | | TTCATTAACACTGGTAGAAGTAGCACAAACTATGCGGACGCT |
| | | GTGGCGGGCCGATTCACCATCTCCAGAGACAACGCCAAGAA |
| | | CACGCTGTATCTTCAAATGAACGGCCTGACAGTCGAGGACA |
| | | CGGCCGTGTATTACTGTTCGACCCTCGACTACTGGGGCCAG |
| | | GGAACCCTGGTCACCGTCTCCTCAG |
| 169 | PMX075 heavy | EVQLVESGGDLVKPGGSLRLSCVASGITFSRYHMSWVRQAPG |
| | chain variable | KGLQWVAFINTGRSSTNYADAVAGRFTISRDNAKNTLYLQMNG |
| | region amino | LTVEDTAVYYCSTLDYWGQGTLVTVSS |
| | acid sequence | |
| 170 | PMX075 light | ATGGCCTGGACCCACCTCCTCCTGAGCCTCCTGGCTCTCTG |
| | chain variable | CACAGATTCTGTGGCCTCCTATGTGCTGACACAGCTGCCATC |
| | region | CAAAAGTGTGACCCTGAAGCAGCCGGCCCACATCACCTGTG |
| | nucleotide | GGGGAGACAACATTGGAAGTAAAAGTGTTCACTGGTACCAG |
| | sequence | CAGAAGCTGGGCCAGGCCCCTATACTGATTATCTATTATGAT |
| | | AACAACAGGCCGACAGGGATCCCTGAGCGATTCTCCGGCGC |
| | | CAACTCGGGGAACACGGCCACCCTGACCATCAGCGGGGCC |
| | | CTGGCCGAGGACGAGGCTGACTATTACTGCCACGTGTGGGA |
| | | CAGCAGTGTTAATGTGTTCGGCGGAGGCACCCATCTGACCG |
| | | TCCTCG |
| 171 | PMX075 light | SYVLTQLPSKSVTLKQPAHITCGGDNIGSKSVHWYQQKLGQAPI |
| | chain variable | LIIYYDNNRPTGIPERFSGANSGNTATLTISGALAEDEADYYCHV |
| | region amino | WDSSVNVFGGGTHLTVL |
| | acid sequence | |
| 172 | PMX075 heavy | GITFSRYH |
| | chain CDR1 | |
| 173 | PMX075 heavy | INTGRSST |
| | chain CDR2 | |
| 174 | PMX075 heavy | STLDY |
| | chain CDR3 | |
| 175 | PMX075 light | NIGSKS |
| | chain CDR1 | |
| 176 | PMX075 light | YDN |
| | chain CDR2 | |
| 177 | PMX075 light | HVWDSSVNV |
| | chain CDR3 | |
| 178 | PMX076 heavy | ATGGAGTCTGCGCTCAGCTGGGTGTTCCTTGTCACTATTTTA |
| | chain variable | AAAGGTGTCCAGGGTGAAGTTCAACTGGTGGAGTCTGGGGG |
| | region | AGACCTGGTGAAGCCTGGGGGGTCCCTGAGACTTTCCTGTG |
| | nucleotide | TGGCCTCTGGAATCACCTTCAGTAGGTACTACATGAACTGGG |
| | sequence | TCCGCCAGGCTCCAGGGAAGGGGCTTCAGTGGGTCGCATA |
| | | CATTGACAGTGGTGGAAGTCCCACAACCTATGCAGACGCTG |
| | | TGAAGGGCCGATTCACCATCTCCAGAGACAACGCCAAGAAA |
| | | GCGCTGTATCTTCAGATGAACAGCCTGAGAGCCGAGGACAC |
| | | GGCCGTATATTACTGTACGAGTTTTGACTACTGGGGCCAGG |
| | | GAACCCTGGTCACCGTCTCCTCAG |
| 179 | PMX076 heavy | EVQLVESGGDLVKPGGSLRLSCVASGITFSRYYMNWVRQAPG |
| | chain variable | KGLQWVAYIDSGGSPTTYADAVKGRFTISRDNAKKALYLQMNS |
| | region amino | LRAEDTAVYYCTSFDYWGQGTLVTVSS |
| | acid sequence | |
| 180 | PMX076 light | ATGGCCTGGACCCACCTCCTCCTGAGCCTCCTGGCTCTCTG |
| | chain variable | CACAGGTTCTGTGGCCTCCTATGTCCTGACACAGCTGCCATC |
| | region | CAAAAATGTGACCCTGAAGCAGCCGGCCCACATCACCTGTG |
| | nucleotide | GGGGAGACAACATTGGAAGTAAAAGTGTTCACTGGTACCAG |
| | sequence | CAGAAGCTGGGCCAGGCCCCTGTACTGATTATCTATTATGAT |
| | | AGCAGCAGGCCGACAGGGATCCCTGAGCGATTCTCCGGCG |
| | | CCAACTCGGGGAACACGGCCACCCTGACCATCAGCGGGGC |
| | | CCTGGCCGAGGACGAGGCTGACTATTTCTGCCAGGTGTGGG |
| | | ACAGCAGTGCTAATGTGTTCGGCGGAGGCACCCATCTGACC |
| | | GTCCTCG |
| 181 | PMX076 light | SYVLTQLPSKNVTLKQPAHITCGGDNIGSKSVHWYQQKLGQAP |
| | chain variable | VLIIYYDSSRPTGIPERFSGANSGNTATLTISGALAEDEADYFCQ |
| | region amino | VWDSSANVFGGGTHLTVL |
| | acid sequence | |
| 182 | PMX076 heavy | GITFSRYY |
| | chain CDR1 | |
| 183 | PMX076 heavy | IDSGGSPT |
| | chain CDR2 | |
| 184 | PMX076 heavy | TSFDY |
| | chain CDR3 | |
| 185 | PMX076 light | NIGSKS |
| | chain CDR1 | |
| 186 | PMX076 light | YDS |
| | chain CDR2 | |
| 187 | PMX076 light | QVWDSSANV |
| | chain CDR3 | |
| 188 | PMX077 heavy | ATGGAGTCTGCGCTCAGCTGGGTGTTCCTTGTCACTATTTTA |
| | chain variable | AAAGGTGTCCAGGGTGAGGTGCAGCTGGTGGAGTCTGGGG |
| | region | GAGACCTGGTGAAGCCTGGGGGGTCCCTGAGACTTTCCTGT |
| | nucleotide | GTGGCCTCTGGAATCACCTTCAGTAGATATTATATGAACTGG |
| | sequence | GTCCGCCAGGCTCCAGGGAAGGGGCTTCAGTGGGTCGCAT |
| | | ACATAAACAGTGGTGGAAGTCCCACAACCTATGCAGACGCT |
| | | GTAAAGGGCCGATTCACCATCTCCAGAGACAACGCCAAGAA |
| | | CACGTTGTATCTTCAGATGAACAGCCTGAGAGCCGAGGACA |
| | | CGGCCGTGTATTACTGTACGAGTTTTGACTACTGGGGCCAG |
| | | GGAACCCTGGTCACCGTCTCCTCAG |
| 189 | PMX077 heavy | EVQLVESGGDLVKPGGSLRLSCVASGITFSRYYMNWVRQAPG |
| | chain variable | KGLQWVAYINSGGSPTTYADAVKGRFTISRDNAKNTLYLQMNS |
| | region amino | LRAEDTAVYYCTSFDYWGQGTLVTVSS |
| | acid sequence | |
| 190 | PMX077 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTATACTGATTATCTATTATGA |
| | | TACCAGCAGGCCGACAGGGATCCCTGAGCGATTCTCCGGCG |
| | | CCAACTCGGGGAACACGGCCACCCTGACCATCAGCGGGGC |
| | | CCTGGCCGAGGACGAGGCTGACTATTACTGCCAGGTGTGGG |
| | | ACAGCAGTGCTAATGTGTTCGGCGGAGGCACCCATCTGACC |
| | | GTCCTCG |
| 191 | PMX077 light | SYVLTQLPSKNVTLKQPAHITCGGDNIGSKSVHWYQQKLGQAPI |
| | chain variable | LIIYYDTSRPTGIPERFSGANSGNTATLTISGALAEDEADYYCQV |
| | region amino | WDSSANVFGGGTHLTVL |
| | acid sequence | |
| 192 | PMX077 heavy | GITFSRYY |
| | chain CDR1 | |
| 193 | PMX077 heavy | INSGGSPT |
| | chain CDR2 | |
| 194 | PMX077 heavy | TSFDY |
| | chain CDR3 | |
| 195 | PMX077 light | NIGSKS |
| | chain CDR1 | |
| 196 | PMX077 light | YDT |
| | chain CDR2 | |
| 197 | PMX077 light | QVWDSSANV |
| | chain CDR3 | |
| 198 | PMX078 heavy | ATGGAGTCTGCGCTCAGCTGGGTGTTCCTTGTCACAATTTTA |
| | chain variable | AAAGGTGTCCAGGGTGAGGTGCAACTGGTGGAGTCTGGGG |
| | region | GAGACCTGGTGAAGCCTGGGGGGTCCCTGAGACTTTCCTGT |
| | nucleotide | GTGGCCTCTGGACTCACCTTCAGTAGGTACCACATGAGCTG |
| | sequence | GATCCGCCAGGCTCCAGGGAAGGGGCTTCAGTGGGTCGCA |
| | | TACATTAGCAGTGGTGGAAGTAGCACAAACTATGCAGGCTCT |
| | | GTGAAGGGCCGATTCACCGTCTCCAGAGACAACGCCAAGAA |
| | | CACTCTGTATCTTCAGATGAACAGTCTGAGAGCCGAAGACAC |
| | | GGCCGTCTATTACTGTACGAGTCTTGACTACTGGGGCCAGG |
| | | GAACCCTGGTCACCGTCTCCTCAG |
| 199 | PMX078 heavy | EVQLVESGGDLVKPGGSLRLSCVASGLTFSRYHMSWIRQAPG |
| | chain variable | KGLQWVAYISSGGSSTNYAGSVKGRFTVSRDNAKNTLYLQMN |
| | region amino | SLRAEDTAVYYCTSLDYWGQGTLVTVSS |
| | acid sequence | |
| 200 | PMX078 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTACTGATTATCTATTATGA |
| | | TAGTAGGAGGCCGACAGGGATCCCTGAGCGATTCTCCGGCG |
| | | CCAACTCGGGGAACACGGCCACCCTGACCATCAGCGGGGC |
| | | CCTGGCCGAGGACGAGGCTGACTATTACTGCCAGGTGTGGG |
| | | ACAGCAGTGATAATGTGTTCGGCGGAGGCACCCATCTGACC |
| | | GTCCTCG |
| 201 | PMX078 light | SYVLTQLPSKNVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSDNVFGGGTHLTVL |
| | acid sequence | |
| 202 | PMX078 heavy | GLTFSRYH |
| | chain CDR1 | |
| 203 | PMX078 heavy | ISSGGSST |
| | chain CDR2 | |
| 204 | PMX078 heavy | TSLDY |
| | chain CDR3 | |
| 205 | PMX078 light | NIGSKS |
| | chain CDR1 | |
| 206 | PMX078 light | YDS |
| | chain CDR2 | |
| 207 | PMX078 light | QVWDSSDNV |
| | chain CDR3 | |
| 208 | PMX079 heavy | ATGGAGTCTGCGCTCAGCTGGGTGTTCCTTGTCACTATTTTA |
| | chain variable | AAAGGTGTCCAGGGTGAGGTGCAGCTGGTGGAGTCTGGGG |
| | region | GAGACCTGGTGAAGCCGGGGGGGTCCCTGAGACTTTCCTGT |
| | nucleotide | GTGGCCTCTGGACTCACCTTCAGTAGATACCACATGAGTTGG |
| | sequence | GTCCGCCAGGCTCCAGGGAAGGGGCTTCAGTGGGTCGCAT |
| | | TCATTAACAGTGGTAGAAGTGACACAACCTATGCAGACGCTG |
| | | TGAAGGGCCGATTCACCATCTCCAGAGACAACGCCAAGAAC |
| | | ACGCTGTATCTTCAGATGAACAGCCTGAGAGCCGAGGACAC |
| | | GGCCGTTTATTACTGTACGACTTTTGACTACTGGGGCCAGGG |
| | | AACCCTGGTCACCGTCTCCTCAG |
| 209 | PMX079 heavy | EVQLVESGGDLVKPGGSLRLSCVASGLTFSRYHMSWVRQAPG |
| | chain variable | KGLQWVAFINSGRSDTTYADAVKGRFTISRDNAKNTLYLQMNS |
| | region amino | LRAEDTAVYYCTTFDYWGQGTLVTVSS |
| | acid sequence | |
| 210 | PMX079 light | ATGGCCTGGACCCACCTCCTCCTGAGCCTCCTGGCTCTCTG |
| | chain variable | CACAGGTTATGTGGCCTCCTATGTGCTGACACAGCTGCCATC |
| | region | CAAAAATGTGACCCTGAAGCAGCCGGCCCACATCACCTGTG |
| | nucleotide | GGGGAGACAACATTGGAAGTAAAAGTGTTCACTGGTACCAG |
| | sequence | CAGAAGCTGGGCCAGGCCCCTGCACTGATTATCTATTATGAT |
| | | AGCAGCAGGCCGACAGGGATCCCTGAGCGATTCTCCGGCG |
| | | CCAACTCGGGGAACACGGCCACCCTGACCATCAGCGGGGC |
| | | CCTGGCCGAGGACGAGGCTGACTATTACTGCCAGGTGTGGG |
| | | ACAGTAGTGCTAATGTGTTCGGCGGAGGCACCCATCTGACC |
| | | GTCCTCG |
| 211 | PMX079 light | SYVLTQLPSKNVTLKQPAHITCGGDNIGSKSVHWYQQKLGQAP |
| | chain variable | ALIIYYDSSRPTGIPERFSGANSGNTATLTISGALAEDEADYYCQ |
| | region amino | VWDSSANVFGGGTHLTVL |
| | acid sequence | |
| 212 | PMX079 heavy | GLTFSRYH |
| | chain CDR1 | |
| 213 | PMX079 heavy | INSGRSDT |
| | chain CDR2 | |
| 214 | PMX079 heavy | TTFDY |
| | chain CDR3 | |
| 215 | PMX079 light | NIGSKS |
| | chain CDR1 | |
| 216 | PMX079 light | YDS |
| | chain CDR2 | |
| 217 | PMX079 light | QVWDSSANV |
| | chain CDR3 | |
| 218 | PMX080 heavy | ATGGAGTCTGCGCTCAGCTGGGTGTTCCTCGTCACTATTTTA |
| | chain variable | AAAGGTGTCCAGGGTGAGGTGCAGTTGGTGGAGTCTGGGG |
| | region | GAGACCTGGTGAAGCCTGGGGGGTCCCTGAGACTTTCCTGT |
| | nucleotide | GTGGCCTCTGGATTAACCTTCAGTAGATATTACATGAACTGG |
| | sequence | GTCCGCCAGGCTCCAGGGAAGGGGCTTCAGTGGGTCGCAT |
| | | ATATTGATAGTGGTGGAAGTCCCACAAACTATGCAGACGCTG |
| | | TGAAGGGCCGATTCACCATCTCCAGAGACAACGCCAAGAAC |
| | | ACACTGTATCTTCAGATGAACAGCCTGAGAGCCGAGGACAC |
| | | GGCCGTATATTATTGTTCGAGTTTTGACTACTGGGGCCAGGG |
| | | AACCCTGGTCACCGTCTCCTCAG |
| 219 | PMX080 heavy | EVQLVESGGDLVKPGGSLRLSCVASGLTFSRYYMNWVRQAPG |
| | chain variable | KGLQWVAYIDSGGSPTNYADAVKGRFTISRDNAKNTLYLQMNS |
| | region amino | LRAEDTAVYYCSSFDYWGQGTLVTVSS |
| | acid sequence | |
| 220 | PMX080 light | ATGGCCTGGACCCACCTCCTCCTGAGCCTCCTGGCTCTCTG |
| | chain variable | CACAGGTTCTGTGGCCTCCTATGTGGTGACACAGCTGCCAT |
| | region | CCAATACTGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTGTGCTGATTATCTATTATG |
| | | ATAGCAGGAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACAGTAGTGCTAATGTGTTCGGCGGAGGCACCCATCTGAC |
| | | CGTCCTCG |
| 221 | PMX080 light | SYVVTQLPSNTVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSANVFGGGTHLTVL |
| | acid sequence | |
| 222 | PMX080 heavy | GLTFSRYY |
| | chain CDR1 | |
| 223 | PMX080 heavy | IDSGGSPT |
| | chain CDR2 | |
| 224 | PMX080 heavy | SSFDY |
| | chain CDR3 | |
| 225 | PMX080 light | NIGSKS |
| | chain CDR1 | |
| 226 | PMX080 light | YDS |
| | chain CDR2 | |
| 227 | PMX080 light | QVWDSSANV |
| | chain CDR3 | |
| 228 | PMX081 heavy | ATGGAGTCTGCGCTCAGCTGGGTGTTCCTTGTCACTATTTTA |
| | chain variable | AAAGGTGTCCAGGGTGAGGTGCAGCTGGTGCAGTCTGGGG |
| | region | GAGACCTGGTGAAGCCTGGGGGGTCCCTGAGACTTTCCTGT |
| | nucleotide | GTGGCCTCTGGATACACCTTCACTAGGTATCACATGAGTTGG |
| | sequence | GTCCGCCAGGCTCCAGGGAAGGGGCTTCAGTGGGTCGCAT |
| | | TCATTAATAGTGATGGAACTGGTATAACCTATGGAGACGCTG |
| | | TGAAGGGCCGATTCACCATCTCCAGAGACAATGCCAAGAAC |
| | | ACGCTGTATCTTCAGATGAACAGCCTGAGAGCCGAGGACAC |
| | | GGCCATCTATTACTGTACGACTTTTGACTCCTGGGGCCAGG |
| | | GAACCCTGGTCACCGTCTCCTCAG |
| 229 | PMX081 heavy | EVQLVQSGGDLVKPGGSLRLSCVASGYTFTRYHMSWVRQAPG |
| | chain variable | KGLQWVAFINSDGTGITYGDAVKGRFTISRDNAKNTLYLQMNSL |
| | region amino | RAEDTAIYYCTTFDSWGQGTLVTVSS |
| | acid sequence | |
| 230 | PMX081 light | ATGGCCTGGACCCACCTCCTCCTGAGCCTCCTGGCTCTCTG |
| | chain variable | CACAGGTTCTGTGGCCTCCTATGTGCTGACACAGCTGCCAT |
| | region | CCAAAAATGTGACCCTGAAGCAGCCGGCCCACATCACCTGT |
| | nucleotide | GGGGGAGACAACATTGGAAGTAAAAGTGTTCACTGGTACCA |
| | sequence | GCAGAAGCTGGGCCAGGCCCCTATACTGATTATCTATTATGA |
| | | TAGCAGGAGGCCGACAGGGATCCCTGAGCGATTCTCCGGC |
| | | GCCAACTCGGGGAACACGGCCACCCTGACCATCAGCGGGG |
| | | CCCTGGCCGAGGACGAGGCTGACTATTACTGCCAGGTGTGG |
| | | GACAGCAGTGGTAATGTGTTCGGCGGAGGCACCCATCTGAC |
| | | CGTCCTCG |
| 231 | PMX081 light | SYVLTQLPSKNVTLKQPAHITCGGDNIGSKSVHWYQQKLGQAPI |
| | chain variable | LIIYYDSRRPTGIPERFSGANSGNTATLTISGALAEDEADYYCQV |
| | region amino | WDSSGNVFGGGTHLTVL |
| | acid sequence | |
| 232 | PMX081 heavy | GYTFTRYH |
| | chain CDR1 | |
| 233 | PMX081 heavy | INSDGTGI |
| | chain CDR2 | |
| 234 | PMX081 heavy | TTFDS |
| | chain CDR3 | |
| 235 | PMX081 light | NIGSKS |
| | chain CDR1 | |
| 236 | PMX081 light | YDS |
| | chain CDR2 | |
| 237 | PMX081 light | QVWDSSGNV |
| | chain CDR3 | |
| 238 | PMX112 light | SYVLTQLPSKSVTLKQPAHITCGGDNIGSKSVHWYQQKLGQAP |
| | chain variable | VLIIYYDSRRPTGIPERFSGANSGNTATLTINGALAEDEADYYCQ |
| | region amino | MWDSSANVFGGGTHLTVL |
| | acid sequence | |
| 239 | PMX113 light | SYVLTQLPFKSVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSVNVFGGGTHLTVL |
| | acid sequence | |
| 240 | PMX114 light | SYVLTQLPSKSVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSANVFGGGTHLTVL |
| | acid sequence | |
| 241 | PMX115 light | SYVLTQVPSKSVTLKQPAHITCGGDNIGGKSVHWYQQKLGQAP |
| | chain variable | LLIIYYDTRRPTGIPERFSGANSGNTATLTISRALAEDEADYYCQ |
| | region amino | VWDSSGNVFGGGTHLTVI |
| | acid sequence | |
| 242 | PMX116 light | SYVLTQLPSKSVTLKQPAHITCGGDNIGSKSVHWYQQKLGQAPI |
| | chain variable | LIIYYDTSRPTGIPERFSGANSGNTATLTISGALAEDEADYYCQV |
| | region amino | WDSSANVFGGGTHLTVL |
| | acid sequence | |
| 243 | PMX0117 light | SYVLTQLPSKSVTLKQPAHITCGGDNIGSKSVHWYQQKLGQAP |
| | chain variable | VLIIYYDSSRPTGIPERFSGANSGNTATLTISGALAEDEADYYCQ |
| | region amino | VWDSSANVFGGGTHLTVL |
| | acid sequence | |
| 244 | PMX118 light | SYVLTQLPSKSVTLKQPAHITCGGDNIGSKSVHWYQQKLGQAP |
| | chain variable | VLIIYYASSRPTGIPERFSGANSGNTATLTISGALAEDEADYYCQ |
| | region amino | VWDSSANVFGGGTHLTVL |
| | acid sequence | |
| 245 | PMX119 light | SYVLTQLPSKSVTLKQPAHITCGGDNIGSKSVHWYQQKLGQAP |
| | chain variable | VLIIYYDSRRPTGIPERFSGANSGNTATLTINGALAEDEADYYCQ |
| | region amino | VWDSSANVFGGGTHLTVL |
| | acid sequence | |
| 246 | PMX120 light | SYVLTQLPSKSVTLKQPAHITCGGDNIGSKSVHWYQQKLGQAP |
| | chain variable | VLIIYYDSSRPTGIPERFSGANSGNTATLTISGALAEDEADYFCQ |
| | region amino | VWDSSANVFGGGTHLTVL |
| | acid sequence | |
| 247 | PMX121 light | SYVLTQLPSKSVTLKQPAHITCGGDNIGSKSVHWYQQKLGQAPI |
| | chain variable | LIIYYDTSRPTGIPERFSGANSGNTATLTISGALAEDEADYYCQV |
| | region amino | WDSSANVFGGGTHLTVL |
| | acid sequence | |
| 248 | PMX122 light | SYVLTQLPSKSVTLKQPAHITCGGDNIGSKSVHWYQQKLGQAP |
| | chain variable | VLIIYYDSRRPTGIPERFSGANSGNTATLTISGALAEDEADYYCQ |
| | region amino | VWDSSDNVFGGGTHLTVL |
| | acid sequence | |
| 249 | PMX123 light | SYVLTQLPSKSVTLKQPAHITCGGDNIGSKSVHWYQQKLGQAP |
| | chain variable | ALIIYYDSSRPTGIPERFSGANSGNTATLTISGALAEDEADYYCQ |
| | region amino | VWDSSANVFGGGTHLTVL |
| | acid sequence | |
| 250 | PMX124 light | SYVLTQLPSKSVTLKQPAHITCGGDNIGSKSVHWYQQKLGQAPI |
| | chain variable | LIIYYDSRRPTGIPERFSGANSGNTATLTISGALAEDEADYYCQV |
| | region amino | WDSSGNVFGGGTHLTVL |
| | acid sequence | |
| 251 | PMX066 heavy | GCGGTGCAGCTGGTGGAGTCAGGGGGAGACCTGGTGAAGC |
| | chain variable | CTGGGGGGTCCCTGAGACTTTCCTGTGTGGCCTCTGGAATC |
| | region | ACCTTCAGTAGGTACCACATGAGTTGGGTCCGCCAGGCTCC |
| | nucleotide | AGGGAAGGGGCTTCAGTGGGTCGCATACATTAATAGTGGTG |
| | sequence | GAAGTGCCACAAGTTATACAGACGCTGTGAAGGGCCGATTC |
| | (without leader) | AGCATCTCCAGAGACAACGGCAAAAACACGCTTTATCTTCAG |
| | | ATGAATAGCCTGAGAGTCGAGGACTCGGCCGTCTATTACTGT |
| | | ACGACCCTTGACCTCTGGGGCCAGGGAACCCTGGTCACCGT |
| | | CTCCTCAG |
| 252 | PMX067 heavy | GAGATACAACTGGTGGAGTCTGGGGGAGACCTGGTGAAGCC |
| | chain variable | TGGGGGGTCCCTGAGACTTTCCTGTGTGGCCTCTGGATTCA |
| | region | CCTTCAGTCGCTACCACATGAGCTGGGTCCGCCAGGCTCCA |
| | nucleotide | GGGAAGGGGCTTCAGTGGGTCGCATATATTAACACTGGTGG |
| | sequence | AAGTAACACAAACTATGCAGACGCTGTGAAGGGCCGATTCA |
| | (without leader) | CCATCTCCAGAGACAACGCCAAGAACACGCTGTATCTTCAGA |
| | | TGAACAGCCTGAGAGCCGAGGACACGGCCGTGTATTACTGT |
| | | ACGACCCTTGACTCCTGGGGCCAGGGAACCCTGGTCACCGT |
| | | CTCCTCAG |
| 253 | PMX068 heavy | GAACTACAGCTGGTGGAGTCTGGGGGAGACCTGGTGAAGC |
| | chain variable | CTGGGGGGTCCCTGAGACTTTCCTGTGTGGCCTCCGGATTC |
| | region | ACCTTCAGTAGATACTACTTGAACTGGGTCCGCCAGGCTCCA |
| | nucleotide | GGGAAGGGGCTTCAGTGGGTCGCATTCATTACCAGTGGTGG |
| | sequence | CAGTAGCACAAACTATGCAGACGCTGTGAAGGGCCGATTCA |
| | (without leader) | CCATCTCCAGAGACAACGCCAAGAACACACTGTATCTTCAGA |
| | | TGAATAGCCTGAGAGCCGAGGACACGGCCGTTTATTACTGT |
| | | ACGAGCTTCGACTCCTGGGGCCAGGGAACCCTGGTCACCGT |
| | | CTCCTCAG |
| 254 | PMX069 heavy | GAGATGCAGCTGGTGGAATCTGGGGGAGCCCTGGTGAAGC |
| | chain variable | CTGGGGGGTCCCTGAGACTCTCCTGTGTGGCCTCTGGATTC |
| | region | ACCTTTAGGAGATACCACATGAGTTGGGTCCGCCAGGCTCC |
| | nucleotide | AGGACAGGGGCTTCAGTGGGTCGCATACATTGACAGTGATG |
| | sequence | GAAGTCCTACAAGTTATGGAGACTCTGTGAAGGGCCGATTCA |
| | (without leader) | CCATCTCCAGAGACAACGCCAAGAATACGCTATTTCTTCAGA |
| | | TGAACAGTCTGAGAGTCGACGACACGGCCGTATATTACTGTA |
| | | CGACTCTTGACTACTGGGGCCGGGGAACCCTGGTCACCGTC |
| | | TCCTCAG |
| 255 | PMX070 heavy | GAGATGCAACTGGTGGAGTCTGGGGGAGACCTGGTGAAGC |
| | chain variable | CTGGGGGGTCCCTGAGACTTTCCTGTGTGGCCTCTGGATTC |
| | region | ACCTTCAGTCGCTACCACATGAGCTGGGTCCGCCAGGCTCC |
| | nucleotide | AGGGAAGGGGCTTCAGTGGGTCGCATACATTAATAGTGGTG |
| | sequence | GAAGTCCCACAAACTATGCAGACGCTGTGAAGGGCCGATTC |
| | (without leader) | ACCATCTCCAGAGACAACGCCAAGAACACACTATATCTTCAG |
| | | ATGAACAGCCTGAGAGCCGAGGACACGGCCGTGTATTACTG |
| | | TACGACCTACGACCAGTGGGGCCAGGGAACCCTGGTCACC |
| | | GTCTCCTCAG |
| 256 | PMX071 heavy | GAGGTGCACCTGGTGGAGTCTGGGGGAGACCTGGTGAAGC |
| | chain variable | CTGGGGGGTCCCTGAGACTTTCCTGTGTGGCCTCTGGAATC |
| | region | ACCTTCAATAGATATTATATGAACTGGGTCCGCCAGACTCCA |
| | nucleotide | GGGAAGGGGCTTCAGTGGGTCGCATACATAGACAGTGGTGG |
| | sequence | AAGTCCCACAACCTATGCAGACGCTGTAAAGGGCCGATTCA |
| | (without leader) | CCATCTCCAGAGACAACGCCAAGAACACGTTGTATCTTCAGA |
| | | TGAACAGCCTGAGAGCCGAGGACACGGCCGTATATTACTGT |
| | | ACGAGTTTTGACTACTGGGGCCAGGGAACCCTGGTCACCGT |
| | | CTCCTCAG |
| 257 | PMX072 heavy | GAGGTGCAGCTGGTGGAGTCTGGGGGAGACCTGGTGAAGC |
| | chain variable | CTGGGGGGTCCCTGAGACTTTCCTGTGTGGCCTCTGGATTC |
| | region | ACCTTCAGTAGGTACCACATGAGCTGGGTCCGCCAGGCTCC |
| | nucleotide | AGGGAAGGGGCTTCAGTGGGTCGCATTCATTAACACTGGTA |
| | sequence | GAAGTAGCACAAACTATGCAGACGCTGTGGCGGGCCGATTC |
| | (without leader) | ACCATCTCCAGAGACAACGCCAAGAACACGCTGTATCTTCAG |
| | | ATGAACAGCCTGAGAGCCGAGGACACGGCCGTGTATTACTG |
| | | TTCGACCCTCGACTACTGGGGCCAGGGAACCCTGGTCACCG |
| | | TCTCCTCAG |
| 258 | PMX073 heavy | GAAGTACAGCTGGTGGAGTCTGGGGGAGACCTGGTGAAGC |
| | chain variable | CTGGGGGGTCCCTGAGACTTTCCTGTGTGGCCTCCGGATTC |
| | region | ACCTTCAGTAGATACTACATGAACTGGGTCCGCCAGGCTCCA |
| | nucleotide | GGGAAGGGGCTTCAGTGGGTCGCATTCATTACCAGTGGTGG |
| | sequence | AAGTAGCACAAGCTATGGAGACGCTGTGAAGGGCCGATTCA |
| | (without leader) | CCATCTCCAGAGACAACGCCAAGAACACGCTGTATCTTCAGA |
| | | TGAACAGCCTGAGAGCCGAGGACACGGCCGTGTATTACTGT |
| | | ACGAGCTTCGACTACTGGGGCCAGGGAACCCTGGTCACCGT |
| | | CTCCTCAG |
| 259 | PMX074 heavy | GAGGTGCAGTTGGTGGAGTCTGGGGGAGACCTGGTGAAGC |
| | chain variable | CTGGGGGGTCCCTGAGACTTTCCTGTGTGGCCTCTGGATTC |
| | region | ACCTTCAGTAGGTATTACATGACCTGGGTCCGCCAGGCTCC |
| | nucleotide | AGGGAAGGGGCTTCAGTGGGTCGCATACATTAACAGTGATA |
| | sequence | GAAGAAGCACAACCTATGCAGACGCTGTGAAGGGCCGATTC |
| | (without leader) | ACCATCTCCAGAGACAACGCCAAAAACACGCTGTATCTTCAG |
| | | ATGAACAGCCTGAGAGCCGAGGACACGGCCGTATATTACTG |
| | | TACGACCCTTGAATATTGGGGCCAGGGAACCCTGGTCACCG |
| | | TCTCCTCAG |
| 260 | PMX075 heavy | GAGGTGCAGTTGGTGGAGTCTGGGGGAGACCTGGTGAAGC |
| | chain variable | CGGGGGGGTCCCTGAGACTTTCCTGTGTGGCCTCCGGAATC |
| | region | ACCTTCAGTAGATATCACATGAGCTGGGTCCGCCAGGCTCC |
| | nucleotide | GGGGAAGGGGCTTCAGTGGGTCGCATTCATTAACACTGGTA |
| | sequence | GAAGTAGCACAAACTATGCGGACGCTGTGGCGGGCCGATTC |
| | (without leader) | ACCATCTCCAGAGACAACGCCAAGAACACGCTGTATCTTCAA |
| | | ATGAACGGCCTGACAGTCGAGGACACGGCCGTGTATTACTG |
| | | TTCGACCCTCGACTACTGGGGCCAGGGAACCCTGGTCACCG |
| | | TCTCCTCAG |
| 261 | PMX076 heavy | GAAGTTCAACTGGTGGAGTCTGGGGGAGACCTGGTGAAGCC |
| | chain variable | TGGGGGGTCCCTGAGACTTTCCTGTGTGGCCTCTGGAATCA |
| | region | CCTTCAGTAGGTACTACATGAACTGGGTCCGCCAGGCTCCA |
| | nucleotide | GGGAAGGGGCTTCAGTGGGTCGCATACATTGACAGTGGTGG |
| | sequence | AAGTCCCACAACCTATGCAGACGCTGTGAAGGGCCGATTCA |
| | (without leader) | CCATCTCCAGAGACAACGCCAAGAAAGCGCTGTATCTTCAGA |
| | | TGAACAGCCTGAGAGCCGAGGACACGGCCGTATATTACTGT |
| | | ACGAGTTTTGACTACTGGGGCCAGGGAACCCTGGTCACCGT |
| | | CTCCTCAG |
| 262 | PMX077 heavy | GAGGTGCAGCTGGTGGAGTCTGGGGGAGACCTGGTGAAGC |
| | chain variable | CTGGGGGGTCCCTGAGACTTTCCTGTGTGGCCTCTGGAATC |
| | region | ACCTTCAGTAGATATTATATGAACTGGGTCCGCCAGGCTCCA |
| | nucleotide | GGGAAGGGGCTTCAGTGGGTCGCATACATAAACAGTGGTGG |
| | sequence | AAGTCCCACAACCTATGCAGACGCTGTAAAGGGCCGATTCA |
| | (without leader) | CCATCTCCAGAGACAACGCCAAGAACACGTTGTATCTTCAGA |
| | | TGAACAGCCTGAGAGCCGAGGACACGGCCGTGTATTACTGT |
| | | ACGAGTTTTGACTACTGGGGCCAGGGAACCCTGGTCACCGT |
| | | CTCCTCAG |
| 263 | PMX078 heavy | GAGGTGCAACTGGTGGAGTCTGGGGGAGACCTGGTGAAGC |
| | chain variable | CTGGGGGGTCCCTGAGACTTTCCTGTGTGGCCTCTGGACTC |
| | region | ACCTTCAGTAGGTACCACATGAGCTGGATCCGCCAGGCTCC |
| | nucleotide | AGGGAAGGGGCTTCAGTGGGTCGCATACATTAGCAGTGGTG |
| | sequence | GAAGTAGCACAAACTATGCAGGCTCTGTGAAGGGCCGATTC |
| | (without leader) | ACCGTCTCCAGAGACAACGCCAAGAACACTCTGTATCTTCAG |
| | | ATGAACAGTCTGAGAGCCGAAGACACGGCCGTCTATTACTG |
| | | TACGAGTCTTGACTACTGGGGCCAGGGAACCCTGGTCACCG |
| | | TCTCCTCAG |
| 264 | PMX079 heavy | GAGGTGCAGCTGGTGGAGTCTGGGGGAGACCTGGTGAAGC |
| | chain variable | CGGGGGGGTCCCTGAGACTTTCCTGTGTGGCCTCTGGACTC |
| | region | ACCTTCAGTAGATACCACATGAGTTGGGTCCGCCAGGCTCC |
| | nucleotide | AGGGAAGGGGCTTCAGTGGGTCGCATTCATTAACAGTGGTA |
| | sequence | GAAGTGACACAACCTATGCAGACGCTGTGAAGGGCCGATTC |
| | (without leader) | ACCATCTCCAGAGACAACGCCAAGAACACGCTGTATCTTCAG |
| | | ATGAACAGCCTGAGAGCCGAGGACACGGCCGTTTATTACTG |
| | | TACGACTTTTGACTACTGGGGCCAGGGAACCCTGGTCACCG |
| | | TCTCCTCAG |
| 265 | PMX080 heavy | GAGGTGCAGTTGGTGGAGTCTGGGGGAGACCTGGTGAAGC |
| | chain variable | CTGGGGGGTCCCTGAGACTTTCCTGTGTGGCCTCTGGATTA |
| | region | ACCTTCAGTAGATATTACATGAACTGGGTCCGCCAGGCTCCA |
| | nucleotide | GGGAAGGGGCTTCAGTGGGTCGCATATATTGATAGTGGTGG |
| | sequence | AAGTCCCACAAACTATGCAGACGCTGTGAAGGGCCGATTCA |
| | (without leader) | CCATCTCCAGAGACAACGCCAAGAACACACTGTATCTTCAGA |
| | | TGAACAGCCTGAGAGCCGAGGACACGGCCGTATATTATTGT |
| | | TCGAGTTTTGACTACTGGGGCCAGGGAACCCTGGTCACCGT |
| | | CTCCTCAG |
| 266 | PMX081 heavy | GAGGTGCAGCTGGTGCAGTCTGGGGGAGACCTGGTGAAGC |
| | chain variable | CTGGGGGGTCCCTGAGACTTTCCTGTGTGGCCTCTGGATAC |
| | region | ACCTTCACTAGGTATCACATGAGTTGGGTCCGCCAGGCTCC |
| | nucleotide | AGGGAAGGGGCTTCAGTGGGTCGCATTCATTAATAGTGATG |
| | sequence | GAACTGGTATAACCTATGGAGACGCTGTGAAGGGCCGATTC |
| | (without leader) | ACCATCTCCAGAGACAATGCCAAGAACACGCTGTATCTTCAG |
| | | ATGAACAGCCTGAGAGCCGAGGACACGGCCATCTATTACTG |
| | | TACGACTTTTGACTCCTGGGGCCAGGGAACCCTGGTCACCG |
| | | TCTCCTCAG |
| 267 | PMX066 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCCGCAGGCCGACAG |
| | sequence | GGATCCCTGAGCGATTCTCCGGCGCCAACTCGGGGAACACG |
| | (without leader) | GCCACCCTGACCATCAACGGGGCCCTGGCCGAGGACGAGG |
| | | CTGACTATTACTGTCAGATGTGGGACAGCAGTGCTAATGTGT |
| | | TCGGCGGAGGCACCCATCTGACCGTCCTCG |
| 268 | PMX067 light | TCCTATGTGCTGACACAGCTGCCATTCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTATTGATTATCTATTATGATAGTAGAAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGTAGTGTTAATGTGT |
| | | TCGGCGGAGGCACCCATCTGACCGTCCTCG |
| 269 | PMX068 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTAATTATCTATTATGATAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 270 | PMX069 light | TCCTATGTGCTGACACAGGTGCCATCCAAAAATGTGACCCTG |
| | chain variable | AAGCAGCCGGCCCACATCACCTGTGGGGGAGACAACATTGG |
| | region | AGGTAAAAGTGTTCACTGGTACCAGCAGAAGCTGGGCCAGG |
| | nucleotide | CCCCTCTACTGATTATCTATTATGATACCAGGAGGCCGACAG |
| | sequence | GGATCCCTGAGCGCTTCTCCGGCGCCAACTCGGGGAACAC |
| | (without leader) | GGCCACCCTGACCATCAGCAGGGCCCTGGCCGAGGACGAG |
| | | GCTGACTATTACTGCCAGGTGTGGGACAGCAGTGGTAATGT |
| | | GTTCGGCGGAGGCACCCATCTGACCGTCATCG |
| 271 | PMX070 light | TCCTATGTGCTGACACAGCTGCCATCCAAAAGTGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACACCAGTGCTAATGTGT |
| | | TCGGCGGAGGCACCCATCTGACCGTCCTCG |
| 272 | PMX071 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTATACTGATTATCTATTATGATACCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 273 | PMX072 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 274 | PMX073 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGCTAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 275 | PMX074 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCCGCAGGCCGACAG |
| | sequence | GGATCCCTGAGCGATTCTCCGGCGCCAACTCGGGGAACACG |
| | (without leader) | GCCACCCTGACCATCAACGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 276 | PMX075 light | TCCTATGTGCTGACACAGCTGCCATCCAAAAGTGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTATACTGATTATCTATTATGATAACAACAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCACGTGTGGGACAGCAGTGTTAATGTGT |
| | | TCGGCGGAGGCACCCATCTGACCGTCCTCG |
| 277 | PMX076 light | TCCTATGTCCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTTCTGCCAGGTGTGGGACAGCAGTGCTAATGTGT |
| | | TCGGCGGAGGCACCCATCTGACCGTCCTCG |
| 278 | PMX077 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTATACTGATTATCTATTATGATACCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGCTAATGTGT |
| | | TCGGCGGAGGCACCCATCTGACCGTCCTCG |
| 279 | PMX078 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTACTGATTATCTATTATGATAGTAGGAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGATAATGTGT |
| | | TCGGCGGAGGCACCCATCTGACCGTCCTCG |
| 280 | PMX079 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGCACTGATTATCTATTATGATAGCAGCAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGTAGTGCTAATGTGT |
| | | TCGGCGGAGGCACCCATCTGACCGTCCTCG |
| 281 | PMX080 light | TCCTATGTGGTGACACAGCTGCCATCCAATACTGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTGTGCTGATTATCTATTATGATAGCAGGAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGTAGTGCTAATGTGT |
| | | TCGGCGGAGGCACCCATCTGACCGTCCTCG |
| 282 | PMX081 light | TCCTATGTGCTGACACAGCTGCCATCCAAAAATGTGACCCTG |
| | chain variable | AAGCAGCCGGCCCACATCACCTGTGGGGGAGACAACATTGG |
| | region | AAGTAAAAGTGTTCACTGGTACCAGCAGAAGCTGGGCCAGG |
| | nucleotide | CCCCTATACTGATTATCTATTATGATAGCAGGAGGCCGACAG |
| | sequence | GGATCCCTGAGCGATTCTCCGGCGCCAACTCGGGGAACACG |
| | (without leader) | GCCACCCTGACCATCAGCGGGGCCCTGGCCGAGGACGAGG |
| | | CTGACTATTACTGCCAGGTGTGGGACAGCAGTGGTAATGTG |
| | | TTCGGCGGAGGCACCCATCTGACCGTCCTCG |
| 283 | PMX006 light | AGCTACGTGCTGACACAGCTGCCTAGCAAGCAAGTGACCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 284 | PMX007 light | AGCTACGTGCTGACACAGCTGCCTAGCAAGGCCGTGACCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 285 | PMX008 light | AGCTACGTGCTGACACAGCTGCCTAGCAAAGAGGTGACCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 286 | PMX009 light | AGCTACGTGCTGACACAGCTGCCTAGCAAGAACGTGGCCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 287 | PMX010 light | AGCTACGTGCTGACACAGCTGCCTAGCAAGTCCGTGACCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 288 | PMX011 light | AGCTACGTGCTGACACAGCTGCCTAGCAAGACCGTGACCCT |
| | chain variable | GAAGCAGCCTGCCCACATCACCTGCGGCGGCGACAACATCG |
| | region | GCAGCAAGAGCGTGCACTGGTATCAGCAGAAGCTGGGCCAA |
| | nucleotide | GCCCCTGTGCTGATCATCTACTACGACAGCAGAAGACCTAC |
| | sequence | CGGCATCCCTGAGAGATTCAGCGGCGCCAACAGCGGCAACA |
| | (codon- | CCGCCACCCTGACCATCAGCGGCGCCCTGGCCGAGGACGA |
| | optimised) | GGCCGACTACTACTGCCAAGTGTGGGACAGCAGCGCCAACG |
| | | TGTTCGGCGGAGGCACCCACCTGACCGTGCTGG |
| 289 | PMX112 light | TCTTACGTGCTGACACAGCTGCCATCTAAGTCCGTGACCCTG |
| | chain variable | AAGCAGCCAGCTCACATCACATGCGGAGGCGACAACATCGG |
| | region | CAGCAAGTCTGTGCATTGGTATCAGCAGAAGCTGGGCCAGG |
| | nucleotide | CTCCCGTGCTGATCATCTACTATGATTCCAGGCGGCCCACC |
| | sequence | GGAATCCCTGAGAGGTTCTCCGGCGCCAACAGCGGCAATAC |
| | (codon- | CGCTACACTGACCATCAACGGCGCCCTGGCTGAGGACGAG |
| | optimised) | GCTGATTACTATTGTCAGATGTGGGACTCCTCCGCCAACGTG |
| | | TTCGGAGGAGGAACACACCTGACCGTGCTGG |
| 290 | PMX113 light | TCCTACGTGCTGACACAGCTGCCATTCAAGAGCGTGACCCT |
| | chain variable | GAAGCAGCCAGCTCACATCACATGCGGAGGCGACAACATCG |
| | region | GCTCTAAGTCCGTGCATTGGTATCAGCAGAAGCTGGGCCAG |
| | nucleotide | GCTCCCGTGCTGATCATCTACTATGATTCTAGGCGGCCCACC |
| | sequence | GGCATCCCTGAGAGGTTCAGCGGCGCCAACTCTGGCAATAC |
| | (codon- | CGCTACACTGACCATCTCCGGCGCCCTGGCTGAGGACGAG |
| | optimised) | GCTGATTACTATTGTCAAGTGTGGGACTCCAGCGTGAACGTG |
| | | TTCGGCGGCGGCACACACCTGACCGTGCTGG |
| 291 | PMX114 light | AGCTACGTGCTGACACAGCTGCCAAGCAAGTCTGTGACCCT |
| | chain variable | GAAGCAGCCAGCTCACATCACATGCGGAGGCGACAACATCG |
| | region | GCTCCAAGAGCGTGCATTGGTATCAGCAGAAGCTGGGCCAG |
| | nucleotide | GCTCCCGTGCTGATCATCTACTATGATTCCAGCAGGCCAACC |
| | sequence | GGAATCCCTGAGCGGTTCTCTGGCGCCAACTCCGGCAATAC |
| | (codon- | CGCTACACTGACCATCTCTGGCGCCCTGGCTGAGGACGAGG |
| | optimised) | CTGATTACTATTGTCAAGTGTGGGACTCTTCCGCTAACGTGT |
| | | TCGGCGGCGGCACACACCTGACCGTGCTGG |
| 292 | PMX115 light | TCCTACGTGCTGACACAGGTGCCATCTAAGTCCGTGACCCT |
| | chain variable | GAAGCAGCCAGCTCACATCACATGCGGAGGCGACAACATCG |
| | region | GAGGCAAGAGCGTGCATTGGTATCAGCAGAAGCTGGGCCAG |
| | nucleotide | GCTCCCCTGCTGATCATCTACTATGATACAAGGCGGCCCAC |
| | sequence | CGGCATCCCTGAGAGATTCAGCGGCGCCAACTCTGGCAATA |
| | (codon- | CCGCTACACTGACCATCTCTCGCGCCCTGGCTGAGGACGAG |
| | optimised) | GCTGATTACTATTGTCAAGTGTGGGACTCCAGCGGAAACGT |
| | | GTTCGGAGGAGGAACACACCTGACCGTGATCG |
| 293 | PMX116 light | TCCTACGTGCTGACACAGCTGCCATCTAAGTCCGTGACCCT |
| | chain variable | GAAGCAGCCAGCTCACATCACATGCGGAGGCGACAACATCG |
| | region | GCAGCAAGTCTGTGCATTGGTATCAGCAGAAGCTGGGCCAG |
| | nucleotide | GCTCCCATCCTGATCATCTACTATGATACATCTAGGCCAACC |
| | sequence | GGAATCCCTGAGCGGTTCTCCGGAGCTAACAGCGGCAATAC |
| | (codon- | CGCTACACTGACCATCTCCGGCGCCCTGGCTGAGGACGAG |
| | optimised) | GCTGATTACTATTGTCAAGTGTGGGACTCCTCCGCCAACGTG |
| | | TTCGGAGGAGGAACACACCTGACCGTGCTGG |
| 294 | PMX117 light | AGCTACGTGCTGACACAGCTGCCAAGCAAGTCTGTGACCCT |
| | chain variable | GAAGCAGCCAGCTCACATCACATGCGGAGGCGACAACATCG |
| | region | GCTCCAAGAGCGTGCATTGGTATCAGCAGAAGCTGGGCCAG |
| | nucleotide | GCTCCCGTGCTGATCATCTACTATGATTCCAGCAGGCCAACC |
| | sequence | GGAATCCCTGAGCGGTTCTCTGGCGCCAACTCCGGCAATAC |
| | (codon- | CGCTACACTGACCATCTCTGGCGCCCTGGCTGAGGACGAGG |
| | optimised) | CTGATTACTATTGTCAAGTGTGGGACTCTTCCGCTAACGTGT |
| | | TCGGCGGCGGCACACACCTGACCGTGCTGG |
| 295 | PMX118 light | AGCTACGTGCTGACACAGCTGCCAAGCAAGTCTGTGACCCT |
| | chain variable | GAAGCAGCCAGCTCACATCACATGCGGAGGCGACAACATCG |
| | region | GCTCCAAGAGCGTGCATTGGTATCAGCAGAAGCTGGGACAG |
| | nucleotide | GCTCCCGTGCTGATCATCTACTATGCTTCCAGCAGGCCAACC |
| | sequence | GGAATCCCTGAGCGGTTCTCTGGCGCCAACTCCGGCAATAC |
| | (codon- | CGCTACACTGACCATCTCTGGCGCCCTGGCTGAGGACGAGG |
| | optimised) | CTGATTACTATTGTCAAGTGTGGGATTCTTCCGCTAACGTGT |
| | | TCGGCGGCGGCACACACCTGACCGTGCTGG |
| 296 | PMX119 light | TCTTACGTGCTGACACAGCTGCCATCTAAGTCCGTGACCCTG |
| | chain variable | AAGCAGCCAGCTCACATCACATGCGGAGGCGACAACATCGG |
| | region | CAGCAAGTCTGTGCATTGGTATCAGCAGAAGCTGGGCCAGG |
| | nucleotide | CTCCCGTGCTGATCATCTACTATGATTCCAGGCGGCCCACC |
| | sequence | GGAATCCCTGAGAGGTTCTCCGGCGCCAACAGCGGCAATAC |
| | (codon- | CGCTACACTGACCATCAACGGCGCCCTGGCTGAGGACGAG |
| | optimised) | GCTGATTACTATTGTCAAGTGTGGGACTCCTCCGCCAACGTG |
| | | TTCGGAGGAGGAACACACCTGACCGTGCTGG |
| 297 | PMX120 light | AGCTACGTGCTGACACAGCTGCCAAGCAAGTCTGTGACCCT |
| | chain variable | GAAGCAGCCAGCTCACATCACATGCGGAGGCGACAACATCG |
| | region | GCTCCAAGAGCGTGCATTGGTATCAGCAGAAGCTGGGCCAG |
| | nucleotide | GCTCCCGTGCTGATCATCTACTATGATTCCAGCAGGCCAACC |
| | sequence | GGAATCCCTGAGCGGTTCTCTGGCGCCAACTCCGGCAATAC |
| | (codon- | CGCTACACTGACCATCTCTGGCGCCCTGGCTGAGGACGAGG |
| | optimised) | CTGATTACTTCTGTCAAGTGTGGGACTCTTCCGCTAACGTGT |
| | | TCGGCGGCGGCACACACCTGACCGTGCTGG |
| 298 | PMX121 light | TCCTACGTGCTGACACAGCTGCCATCTAAGTCCGTGACCCT |
| | chain variable | GAAGCAGCCAGCTCACATCACATGCGGAGGCGACAACATCG |
| | region | GCAGCAAGTCTGTGCATTGGTATCAGCAGAAGCTGGGCCAG |
| | nucleotide | GCTCCCATCCTGATCATCTACTATGATACATCTAGGCCAACC |
| | sequence | GGAATCCCTGAGCGGTTCTCCGGAGCTAACAGCGGCAATAC |
| | (codon- | CGCTACACTGACCATCTCCGGCGCCCTGGCTGAGGACGAG |
| | optimised) | GCTGATTACTATTGTCAAGTGTGGGACTCCTCCGCCAACGTG |
| | | TTCGGAGGAGGAACACACCTGACCGTGCTGG |
| 299 | PMX122 light | TCCTACGTGCTGACACAGCTGCCATCTAAGTCCGTGACCCT |
| | chain variable | GAAGCAGCCAGCTCACATCACATGCGGAGGCGACAACATCG |
| | region | GCAGCAAGTCTGTGCATTGGTATCAGCAGAAGCTGGGCCAG |
| | nucleotide | GCTCCCGTGCTGATCATCTACTATGATTCTAGGCGGCCCACC |
| | sequence | GGCATCCCTGAGAGGTTCTCCGGCGCCAACAGCGGCAATAC |
| | (codon- | CGCTACACTGACCATCTCCGGCGCCCTGGCTGAGGACGAG |
| | optimised) | GCTGATTACTATTGTCAAGTGTGGGACTCCAGCGATAACGTG |
| | | TTCGGCGGCGGCACACACCTGACCGTGCTGG |
| 300 | PMX123 light | AGCTACGTGCTGACACAGCTGCCAAGCAAGTCTGTGACCCT |
| | chain variable | GAAGCAGCCAGCTCACATCACATGCGGAGGCGACAACATCG |
| | region | GCTCCAAGAGCGTGCATTGGTATCAGCAGAAGCTGGGACAG |
| | nucleotide | GCTCCCGCTCTGATCATCTACTATGATTCCAGCAGGCCAACC |
| | sequence | GGAATCCCTGAGCGGTTCTCTGGCGCCAACTCCGGCAATAC |
| | (codon- | CGCTACACTGACCATCTCTGGCGCCCTGGCTGAGGACGAGG |
| | optimised) | CTGATTACTATTGTCAAGTGTGGGACTCTTCCGCTAACGTGT |
| | | TCGGCGGCGGCACACACCTGACCGTGCTGG |
| 301 | PMX124 light | TCCTACGTGCTGACACAGCTGCCATCTAAGTCCGTGACCCT |
| | chain variable | GAAGCAGCCAGCTCACATCACATGCGGAGGCGACAACATCG |
| | region | GCAGCAAGTCTGTGCATTGGTATCAGCAGAAGCTGGGCCAG |
| | nucleotide | GCTCCCATCCTGATCATCTACTATGATTCTAGGCGGCCCACC |
| | sequence | GGCATCCCTGAGAGGTTCTCCGGCGCCAACAGCGGCAATAC |
| | (codon- | CGCTACACTGACCATCTCCGGCGCCCTGGCTGAGGACGAG |
| | optimised) | GCTGATTACTATTGTCAAGTGTGGGACTCCAGCGGAAACGT |
| | | GTTCGGAGGAGGAACACACCTGACCGTGCTGG |

TABLE 3
VH and VL gene usage

| | | |
|---|---|---|
| Antibody | V gene | J gene |

| clone | HC | LC | HC | LC |
|---|---|---|---|---|
| PMX066 | V 3-5 | V 3-3 | J 2 | J 3 |
| PMX067 | V 3-5 | V 3-3 | J 4 | J 3 |
| PMX068 | V 3-5 | V 3-3 | J 4 | J 3 |
| PMX069 | V 3-5 | V 3-3 | J 4 | J 3 |
| PMX070 | V 3-5 | V 3-3 | J 4 | J 3 |
| PMX071 | V 3-5 | V 3-3 | J 4 | J 3 |
| PMX072 | V 3-5 | V 3-3 | J 4 | J 3 |
| PMX073 | V 3-5 | V 3-3 | J 4 | J 3 |
| PMX074 | V 3-5 | V 3-3 | J 4 | J 3 |
| PMX075 | V 3-5 | V 3-3 | J 4 | J 3 |
| PMX076 | V 3-5 | V 3-3 | J 4 | J 3 |
| PMX077 | V 3-5 | V 3-3 | J 4 | J 3 |
| PMX078 | V 3-5 | V 3-3 | J 4 | J 3 |
| PMX079 | V 3-5 | V 3-3 | J 4 | J 3 |
| PMX080 | V 3-5 | V 3-3 | J 4 | J 3 |
| PMX081 | V 3-5 | V 3-3 | J 4 | J 3 |

TABLE 4
Summary of functional data for antibodies

| | | | | | Ex vivo specific | |
|---|---|---|---|---|---|---|
| | ADCC | | CDC | | B cell depletion | |
| | Maximum | | Maximum | | Maximum | Std |
| Antibody | killing | EC₅₀ | killing | EC₅₀ | killing | Deviation |
| clone | (%) | (nM) | (%) | (nM) | (%) | (%) |
| 4E1-7 | 32 | 4.32 | 0.8 | NA | 30 | 13 |
| PMX003 | 34 | 0.89 | 90.4 | 25.03 | 54 | 2 |
| PMX066 | 47 | 0.15 | 98.5 | 12.14 | 44 | 14 |
| PMX067 | 50 | 0.13 | 97.8 | 11.32 | 62 | 5 |
| PMX069 | 38 | 0.09 | 98.2 | 7.60 | 52 | 1 |
| PMX070 | 51 | 0.29 | 97.1 | 11.17 | 66 | 1 |
| PMX071 | 41 | 0.15 | 94.3 | 28.95 | 50 | 10 |
| PMX072 | 50 | 0.18 | 91.6 | 19.62 | 62 | 4 |
| PMX073 | 48 | 0.21 | 96.3 | 11.08 | 68 | 3 |
| PMX074 | 51 | 0.12 | 98.9 | 6.80 | 68 | 4 |
| PMX075 | 46 | 0.14 | 98.9 | 6.02 | 51 | 4 |
| PMX076 | 49 | 0.14 | 98.3 | 7.76 | 54 | 6 |
| PMX077 | 42 | 0.13 | 98.7 | 9.84 | 62 | 0 |
| PMX078 | 40 | 0.22 | 98.8 | 9.08 | 44 | 25 |
| PMX079 | 52 | 0.12 | 96.7 | 16.39 | 67 | 11 |
| PMX080 | 50 | 0.13 | 96.2 | 21.92 | 56 | 1 |
| PMX081 | 51 | 0.16 | 98.5 | 10.55 | 56 | 2 |

TABLE 5
Developability profile

| | | SDS-PAGE | | | | | Diameter |
|---|---|---|---|---|---|---|---|
| Antibody | Titre* | Non | | % Purity by | Tm1 | Tagg | @25° C. |
| clone | (mg/L) | reduced | Reduced | HPLC-SEC | (° C.) | (° C.) | (nm) |
| PMX003 | 125 | One single | Two single | 99.05% | 62.8 ± | 62.8 | 10.79 ± |
| | | band ~150 | bands ~50 | monomer | 0.07 | | 2.80 |
| | | kDa | kDa | | | | |
| | | | and ~25 | | | | |
| | | | kDa | | | | |
| PMX066 | 150 | One single | Two single | 96.98% | 62.1 ± | Estimated ~63 | 10.08 ± |
| | | band ~150 | bands ~50 | monomer | 0.11 | | 2.76 |
| | | kDa | kDa | | | | |
| | | | and ~25 | | | | |
| | | | kDa | | | | |
| PMX067 | 107 | One single | Two single | 98.31% | 64.9 ± | Estimated ~65 | 10.10 ± |
| | | band ~150 | bands ~50 | monomer | 0.05 | | 2.15 |
| | | kDa | kDa | | | | |
| | | | and ~25 | | | | |
| | | | kDa | | | | |
| PMX069 | 199 | One single | Two single | 96.9% | 59.5 ± | 58.5 | 9.64 ± |
| | | band ~150 | bands ~50 | monomer | 0.03 | | 3.05 |
| | | kDa | kDa | | | | |
| | | | and ~25 | | | | |
| | | | kDa | | | | |
| PMX070 | 190 | One single | Two single | 97% | 63.7 ± | 55.5 ± | 11.03 ± |
| | | band ~150 | bands ~50 | monomer | 0.21 | 0.34 | 2.75 |
| | | kDa | kDa | | | | |
| | | | and ~25 | | | | |
| | | | kDa | | | | |
| PMX071 | 106 | One single | Two single | 96.59% | 60.3 ± | Estimated ~62 | 10.25 ± |
| | | band ~150 | bands ~50 | monomer | 0.28 | | 2.83 |
| | | kDa | kDa | | | | |
| | | | and ~25 | | | | |
| | | | kDa | | | | |
| PMX072 | 116 | One single | Two single | 98.83% | 55.3 ± | 53.8 | 10.93 ± |
| | | band ~150 | bands ~50 | monomer | 0.29 | | 2.99 |
| | | kDa | kDa | | | | |
| | | | and ~25 | | | | |
| | | | kDa | | | | |
| PMX073 | 85 | One single | Two single | 98.82% | 59.6 ± | Estimated ~60 | 9.86 ± |
| | | band ~150 | bands ~50 | monomer | 0.17 | | 2.15 |
| | | kDa | kDa | | | | |
| | | | and ~25 | | | | |
| | | | kDa | | | | |
| PMX074 | 159 | One single | Two single | N/A | 62.1 ± | 62.2 | 10.40 ± |
| | | band ~150 | bands ~50 | | 0.17 | | 2.53 |
| | | kDa | kDa | | | | |
| | | | and ~25 | | | | |
| | | | kDa | | | | |
| PMX075 | 100 | One single | Two single | 95.17% | 57.4 ± | 55.4 | 12.02 ± |
| | | band ~150 | bands ~50 | monomer | 0.74 | | 3.83 |
| | | kDa | kDa | | | | |
| | | | and ~25 | | | | |
| | | | kDa | | | | |
| PMX076 | 110 | One single | Two single | 99.44% | 57.9 ± | Estimated ~60 | 9.86 ± |
| | | band ~150 | bands ~50 | monomer | 0.23 | | 2.35 |
| | | kDa | kDa | | | | |
| | | | and ~25 | | | | |
| | | | kDa | | | | |
| PMX077 | 121 | One single | Two single | 98.85% | 61.5 ± | 60.5 | 10.32 ± |
| | | band ~150 | bands ~50 | monomer | 0.16 | | 2.02 |
| | | kDa | kDa | | | | |
| | | | and ~25 | | | | |
| | | | kDa | | | | |
| PMX078 | 97 | One single | Two single | 98.5% | Estimated ~65 | 58 | 9.99 ± |
| | | band ~150 | bands ~50 | monomer | | | 2.23 |
| | | kDa | kDa | | | | |
| | | | and ~25 | | | | |
| | | | kDa | | | | |
| PMX079 | 110 | One single | Two single | 99.31% | 60.7 ± | 59.9 | 10.20 ± |
| | | band ~150 | bands ~50 | monomer | 0.03 | | 1.91 |
| | | kDa | kDa | | | | |
| | | | and ~25 | | | | |
| | | | kDa | | | | |
| PMX080 | 97 | One single | Two single | 99.48% | 59.3 ± | 58 | 10.15 ± |
| | | band ~150 | bands ~50 | monomer | 0.31 | | 2.69 |
| | | kDa | kDa | | | | |
| | | | and ~25 | | | | |
| | | | kDa | | | | |
| PMX081 | 128 | One single | Two single | 99.13% | 61.8 ± | 61.4 | 9.59 ± |
| | | band ~150 | bands ~50 | monomer | 0.08 | | 3.32 |
| | | kDa | kDa | | | | |
| | | | and ~25 | | | | |
| | | | kDa | | | | |
| PMX112 | 80 | One single | Two single | 98.66% | 63.7 ± | 57.8 ± | 10.84 ± |
| | | band ~150 | bands ~50 | monomer | 1.64 | 0.41 | 2.6 |
| | | kDa | kDa | | | | |
| | | | and ~25 | | | | |
| | | | kDa | | | | |
| PMX115 | 331 | One single | Two single | 99% | 60.9 ± | 55.2 ± | 10.46 ± |
| | | band ~150 | bands ~50 | monomer | 0.57 | 0.42 | 1.75 |
| | | kDa | kDa | | | | |
| | | | and ~25 | | | | |
| | | | kDa | | | | |
| PMX122 | 93 | One single | Two single | 97% | 58.7 ± | 57.9 ± | 10.73 ± |
| | | band ~150 | bands ~50 | monomer | 15 | 0.09 | 2.87 |
| | | kDa | kDa | | | | |
| | | | and ~25 | | | | |
| | | | kDa | | | | |
| PMX124 | 139 | One single | Two single | 98% | 63.2 ± | 55.8 ± | 10.66 ± |
| | | band ~150 | bands ~50 | monomer | 0.25 | 0.13 | 2.21 |
| | | kDa | kDa | | | | |
| | | | and ~25 | | | | |
| | | | kDa | | | | |

*average of 3 production batches

1-32. (canceled)

33. An isolated canine antibody or antigen-binding portion thereof which binds canine CD20 wherein the antibody or antigen-binding portion thereof comprises
a) a heavy chain (HC) complementarity-determining region (CDR)1 sequence comprising SEQ ID NO: 27 or an amino acid sequence which has 1 or 2 amino acid differences compared to SEQ ID NO: 27;
b) a HC CDR2 sequence comprising SEQ ID NO: 28 or an amino acid sequence which has 1, 2, 3 or 4 amino acid differences compared to SEQ ID NO: 28;
c) a HC CDR3 sequence comprising SEQ ID NO: 29 or an amino acid sequence which has 1 or 2 amino acid differences compared to SEQ ID NO: 29;
d) a light chain (LC) CDR1 sequence comprising SEQ ID NO: 30 or an amino acid sequence which has 1 amino acid difference compared to SEQ ID NO: 30;
e) a LC CDR2 sequence comprising SEQ ID NO: 31 or an amino acid sequence which has 1 amino acid difference compared to SEQ ID NO: 31; and
f) a LC CDR3 sequence comprising SEQ ID NO: 32 or an amino acid sequence which has 1 or 2 amino acid differences compared to SEQ ID NO: 32.

34. The antibody or antigen-binding portion thereof of claim 33, wherein the antibody or antigen-binding portion thereof has
i) a HC CDR1 sequence comprising SEQ ID NO: 7, a HC CDR2 sequence comprising SEQ ID NO: 8, a HC CDR3 sequence comprising SEQ ID NO: 9, a LC CDR1 sequence comprising SEQ ID NO: 10, a LC CDR2 sequence comprising SEQ ID NO: 11 and a LC CDR3 sequence comprising SEQ ID NO: 12;
ii) a HC CDR1 sequence comprising SEQ ID NO: 17, a HC CDR2 sequence comprising SEQ ID NO: 18, a HC CDR3 sequence comprising SEQ ID NO: 19, a LC CDR1 sequence comprising SEQ ID NO: 20, a LC CDR2 sequence comprising SEQ ID NO: 21 and a LC CDR3 sequence comprising SEQ ID NO: 22;
iii) a HC CDR1 sequence comprising SEQ ID NO: 37, a HC CDR2 sequence comprising SEQ ID NO: 38, a HC CDR3 sequence comprising SEQ ID NO: 39, a LC CDR1 sequence comprising SEQ ID NO: 40, a LC CDR2 sequence comprising SEQ ID NO: 41 and a LC CDR3 sequence comprising SEQ ID NO: 42;
iv) a HC CDR1 sequence comprising SEQ ID NO: 47, a HC CDR2 sequence comprising SEQ ID NO: 48, a HC CDR3 sequence comprising SEQ ID NO: 49, a LC CDR1 sequence comprising SEQ ID NO: 50, a LC CDR2 sequence comprising SEQ ID NO: 51 and a LC CDR3 sequence comprising SEQ ID NO: 52;
v) a HC CDR1 sequence comprising SEQ ID NO: 82, a HC CDR2 sequence comprising SEQ ID NO: 83, a HC CDR3 sequence comprising SEQ ID NO: 84, a LC CDR1 sequence comprising SEQ ID NO: 85, a LC CDR2 sequence comprising SEQ ID NO: 86 and a LC CDR3 sequence comprising SEQ ID NO: 87;
vi) a HC CDR1 sequence comprising SEQ ID NO: 92, a HC CDR2 sequence comprising SEQ ID NO: 93, a HC CDR3 sequence comprising SEQ ID NO: 94, a LC CDR1 sequence comprising SEQ ID NO: 95, a LC CDR2 sequence comprising SEQ ID NO: 96 and a LC CDR3 sequence comprising SEQ ID NO: 97;
vii) a HC CDR1 sequence comprising SEQ ID NO: 102, a HC CDR2 sequence comprising SEQ ID NO: 103, a HC CDR3 sequence comprising SEQ ID NO: 104, a LC CDR1 sequence comprising SEQ ID NO: 105, a LC CDR2 sequence comprising SEQ ID NO: 106 and a LC CDR3 sequence comprising SEQ ID NO: 107;
viii) a HC CDR1 sequence comprising SEQ ID NO: 112, a HC CDR2 sequence comprising SEQ ID NO: 113, a HC CDR3 sequence comprising SEQ ID NO: 114, a LC CDR1 sequence comprising SEQ ID NO: 115, a LC CDR2 sequence comprising SEQ ID NO: 116 and a LC CDR3 sequence comprising SEQ ID NO: 117;
ix) a HC CDR1 sequence comprising SEQ ID NO: 122, a HC CDR2 sequence comprising SEQ ID NO: 123, a HC CDR3 sequence comprising SEQ ID NO: 124, a LC CDR1 sequence comprising SEQ ID NO: 125, a LC CDR2 sequence comprising SEQ ID NO: 126 and a LC CDR3 sequence comprising SEQ ID NO: 127;
x) a HC CDR1 sequence comprising SEQ ID NO: 132, a HC CDR2 sequence comprising SEQ ID NO: 133, a HC CDR3 sequence comprising SEQ ID NO: 134, a LC CDR1 sequence comprising SEQ ID NO: 135, a LC CDR2 sequence comprising SEQ ID NO: 136 and a LC CDR3 sequence comprising SEQ ID NO: 137;
xi) a HC CDR1 sequence comprising SEQ ID NO: 142, a HC CDR2 sequence comprising SEQ ID NO: 143, a HC CDR3 sequence comprising SEQ ID NO: 144, a LC CDR1 sequence comprising SEQ ID NO: 145, a LC CDR2 sequence comprising SEQ ID NO: 146 and a LC CDR3 sequence comprising SEQ ID NO: 147;
xii) a HC CDR1 sequence comprising SEQ ID NO: 152, a HC CDR2 sequence comprising SEQ ID NO: 153, a HC CDR3 sequence comprising SEQ ID NO: 154, a LC CDR1 sequence comprising SEQ ID NO: 155, a LC CDR2 sequence comprising SEQ ID NO: 156 and a LC CDR3 sequence comprising SEQ ID NO: 157;
xii) a HC CDR1 sequence comprising SEQ ID NO: 162, a HC CDR2 sequence comprising SEQ ID NO: 163, a HC CDR3 sequence comprising SEQ ID NO: 164, a LC CDR1 sequence comprising SEQ ID NO: 165, a LC CDR2 sequence comprising SEQ ID NO: 166 and a LC CDR3 sequence comprising SEQ ID NO: 167;
xiii) a HC CDR1 sequence comprising SEQ ID NO: 172, a HC CDR2 sequence comprising SEQ ID NO: 173, a HC CDR3 sequence comprising SEQ ID NO: 174, a LC CDR1 sequence comprising SEQ ID NO: 175, a LC CDR2 sequence comprising SEQ ID NO: 176 and a LC CDR3 sequence comprising SEQ ID NO: 177;
xiv) a HC CDR1 sequence comprising SEQ ID NO: 182, a HC CDR2 sequence comprising SEQ ID NO: 183, a HC CDR3 sequence comprising SEQ ID NO: 184, a LC CDR1 sequence comprising SEQ ID NO: 185, a LC CDR2 sequence comprising SEQ ID NO: 186 and a LC CDR3 sequence comprising SEQ ID NO: 187;
xv) a HC CDR1 sequence comprising SEQ ID NO: 192, a HC CDR2 sequence comprising SEQ ID NO: 193, a HC CDR3 sequence comprising SEQ ID NO: 194, a LC CDR1 sequence comprising SEQ ID NO: 195, a LC CDR2 sequence comprising SEQ ID NO: 196 and a LC CDR3 sequence comprising SEQ ID NO: 197;
xvi) a HC CDR1 sequence comprising SEQ ID NO: 202, a HC CDR2 sequence comprising SEQ ID NO: 203, a HC CDR3 sequence comprising SEQ ID NO: 204, a LC CDR1 sequence comprising SEQ ID NO: 205, a LC CDR2 sequence comprising SEQ ID NO: 206 and a LC CDR3 sequence comprising SEQ ID NO: 207;
xvii) a HC CDR1 sequence comprising SEQ ID NO: 212, a HC CDR2 sequence comprising SEQ ID NO: 213, a HC CDR3 sequence comprising SEQ ID NO: 214, a LC CDR1 sequence comprising SEQ ID NO: 215, a LC CDR2 sequence comprising SEQ ID NO: 216 and a LC CDR3 sequence comprising SEQ ID NO: 217;
xviii) a HC CDR1 sequence comprising SEQ ID NO: 222, a HC CDR2 sequence comprising SEQ ID NO: 223, a HC CDR3 sequence comprising SEQ ID NO: 224, a LC CDR1 sequence comprising SEQ ID NO: 225, a LC CDR2 sequence comprising SEQ ID NO: 226 and a LC CDR3 sequence comprising SEQ ID NO: 227; or
xix) a HC CDR1 sequence comprising SEQ ID NO: 232, a HC CDR2 sequence comprising SEQ ID NO: 233, a HC CDR3 sequence comprising SEQ ID NO: 234, a LC CDR1 sequence comprising SEQ ID NO: 235, a LC CDR2 sequence comprising SEQ ID NO: 236 and a LC CDR3 sequence comprising SEQ ID NO: 237.

35. The antibody or antigen-binding portion thereof of claim 33, wherein the antibody or antigen-binding portion thereof comprises
(a) a HC variable region sequence comprising SEQ ID NO: 24 or a sequence with at least 75%, 80%, 85% or 90% sequence identity thereto; and
(b) a LC variable region sequence comprising SEQ ID NO: 26 or a sequence with at least 70%, 75%, 80%, 85%, 90% or 95% sequence identity thereto; for example a LC variable region sequence comprising SEQ ID NO: 53, 54, 55, 56, 57 or 58.

36. The antibody or antigen-binding portion thereof of claim 35, wherein the antibody or antigen-binding portion thereof has
a) a HC variable region sequence comprising SEQ ID NO: 4 and a LC variable region sequence comprising SEQ ID NO: 6;
b) a HC variable region sequence comprising SEQ ID NO: 14 and a LC variable region sequence comprising SEQ ID NO: 16;
c) a HC variable region sequence comprising SEQ ID NO: 34 and a LC variable region sequence comprising SEQ ID NO: 36;
d) a HC variable region sequence comprising SEQ ID NO: 44 and a LC variable region sequence comprising SEQ ID NO: 46;
e) a HC variable region sequence comprising SEQ ID NO: 79 and a LC variable region sequence comprising SEQ ID NO: 81 or a LC variable region sequence comprising SEQ ID NO: 238;
f) a HC variable region sequence comprising SEQ ID NO: 89 and a LC variable region sequence comprising SEQ ID NO: 91 or a LC variable region sequence comprising SEQ ID NO: 239;
g) a HC variable region sequence comprising SEQ ID NO: 99 and a LC variable region sequence comprising SEQ ID NO: 101 or a LC variable region sequence comprising SEQ ID NO: 240;
h) a HC variable region sequence comprising SEQ ID NO: 109 and a LC variable region sequence comprising SEQ ID NO: 111 or a LC variable region sequence comprising SEQ ID NO: 241;
i) a HC variable region sequence comprising SEQ ID NO: 119 and a LC variable region sequence comprising SEQ ID NO: 121;
j) a HC variable region sequence comprising SEQ ID NO: 129 and a LC variable region sequence comprising SEQ ID NO: 131 or a LC variable region sequence comprising SEQ ID NO: 242;
k) a HC variable region sequence comprising SEQ ID NO: 139 and a LC variable region sequence comprising SEQ ID NO: 141 or a LC variable region sequence comprising SEQ ID NO: 243;
l) a HC variable region sequence comprising SEQ ID NO: 149 and a LC variable region sequence comprising SEQ ID NO: 151 or a LC variable region sequence comprising SEQ ID NO: 244;
m) a HC variable region sequence comprising SEQ ID NO: 159 and a LC variable region sequence comprising SEQ ID NO: 161 or a LC variable region sequence comprising SEQ ID NO: 245;
n) a HC variable region sequence comprising SEQ ID NO: 169 and a LC variable region sequence comprising SEQ ID NO: 171;
o) a HC variable region sequence comprising SEQ ID NO: 179 and a LC variable region sequence comprising SEQ ID NO: 181 or a LC variable region sequence comprising SEQ ID NO: 246;
p) a HC variable region sequence comprising SEQ ID NO: 189 and a LC variable region sequence comprising SEQ ID NO: 191 or a LC variable region sequence comprising SEQ ID NO: 247;
q) a HC variable region sequence comprising SEQ ID NO: 199 and a LC variable region sequence comprising SEQ ID NO: 201 a LC variable region sequence comprising SEQ ID NO: 248;
r) a HC variable region sequence comprising SEQ ID NO: 209 and a LC variable region sequence comprising SEQ ID NO: 211 a LC variable region sequence comprising SEQ ID NO: 249;
s) a HC variable region sequence comprising SEQ ID NO: 219 and a LC variable region sequence comprising SEQ ID NO: 221; or
t) a HC variable region sequence comprising SEQ ID NO: 229 and a LC variable region sequence comprising SEQ ID NO: 231 a LC variable region sequence comprising SEQ ID NO: 250.

37. The antibody or antigen-binding portion thereof of claim 33, which is an scFv, Fv, heavy chain or single domain antibody.

38. The antibody or antigen-binding portion thereof of claim 33, which is conjugated to a therapeutic moiety.

39. The antibody or antigen-binding portion thereof of claim 38, wherein the therapeutic moiety is a second antibody or antigen-binding portion thereof.

40. The antibody or antigen-binding portion thereof of claim 39, wherein the second antibody or antigen-binding portion thereof binds to a different target.

41. The antibody or antigen-binding portion thereof of claim 33, which is conjugated to a further moiety selected from a half life extending moiety, label, cytotoxin, liposome, nanoparticle or radioisotope.

42. The antibody or antigen-binding portion thereof of claim 33, which is a-fucosylated.

43. A pharmaceutical composition comprising an antibody or antigen-binding portion thereof of claim 33 and a pharmaceutically acceptable carrier.

44. A method of treating a condition mediated by B-cells in a canine subject in need thereof, the method comprising administering to the canine subject an effective amount of the antibody or antigen-binding portion thereof of claim 33.

45. The method of claim 44, wherein the disease mediated by B-cells is selected from the group consisting of: B cell lymphoma, leukemia and immune mediated disease.

46. A nucleic acid sequence that encodes an antibody or antigen-binding portion thereof of claim 33.

47. A vector comprising a nucleic acid sequence encoding the antibody or antigen-binding portion thereof of claim 33.

48. A host cell comprising the nucleic acid sequence of claim 46.

49. A kit comprising an antibody or antigen-binding portion thereof of claim 33 and instructions for use.

50. A method for making a canine antibody that binds CD20 comprising culturing the isolated host cell of claim 48 and recovering said antibody.

51. A method of inhibiting canine tumor growth or metastasis comprising contacting a canine tumor cell with an effective amount of the antibody or antigen-binding portion thereof of claim 33.

52. A method of killing a canine tumor cell expressing CD20, comprising contacting the cell with the antibody or antigen-binding portion of claim 33, such that killing of the cell expressing CD20 occurs.

53. An antibody or antigen binding portion thereof which binds to one or more residues within a canine CD20 epitope comprising the following amino residues ENLNLIKAPM (SEQ ID NO: 303, amino acids 150-159 in SEQ ID NO: 2).


SEQUENCE LISTING (365 sequences)

SEQ ID NO: 1 - DNA, 894 bp, Canis sp.
atgacaacacccagaaattcaatgagtggaactctcccggtagatcctatgaaaagccctactgccatgtatcctgttcaaaaaataattcccaaaaggatgccttcagtggtgggccctacacaaaacttcttcatgagggaatctaagacactgggggctgtccagattatgaatgggctcttccacattgccctaggcagcctcctgatgattcacacggatgtctatgcgcccatctgtataactatgtggtaccctctctggggaggcattatgttcatcatttctggatcactcctggcagcagcggacaaaaaccccaggaagagtttggtcaaaggaaaaatgataatgaactcattgagcctctttgctgctatttctggaataatttttttgatcatggacatatttaatattaccatttcccatttttttaaaatggagaatttgaatcttattaaagctcccatgccatatgttgacatacacaactgtgacccagctaacccctctgagaaaaactctttatctatacaatattgtggcagcatacgatctgttttcttgggcgtttttgctgtgatggtgatctttacctttttccagaaacttgtgacagctggcattgttgagaatgaatggaaaaaactgtgctctaaacctaaatctgatgtagttgttctgttagctgctgaagaaaaaaaagaacagccgattgaaacaacagaagaaatggttgagctgactgaaatagcttcccaaccaaagaaagaagaagacattgaaattattccagtccaagaagaagaagaggaactggaaataaactttgcagaacctccccaggagcaggaatcttcaccaatagaaaacgacagcatcccttaa

SEQ ID NO: 2 - protein, 297 aa, Canis sp.
MTTPRNSMSGTLPVDPMKSPTAMYPVQKIIPKRMPSVVGPTQNFFMRESKTLGAVQIMNGLFHIALGSLLMIHTDVYAPICITMWYPLWGGIMFIISGSLLAAADKNPRKSLVKGKMIMNSLSLFAAISGIIFLIMDIFNITISHFFKMENLNLIKAPMPYVDIHNCDPANPSEKNSLSIQYCGSIRSVFLGVFAVMVIFTFFQKLVTAGIVENEWKKLCSKPKSDVVVLLAAEEKKEQPIETTEEMVELTEIASQPKKEEDIEIIPVQEEEEELEINFAEPPQEQESSPIENDSIP

SEQ ID NO: 3 - DNA, 337 bp, Canis sp.
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaatagtggtggaagtagcacaacctatgcagacgctgtgaagggccgattcaccatctccagagacaacgccaagaaaacgctgtatcttcagatgaacagcctgagagccgaggacacggccgtgtattactgttcgacttttgactactggggccagggaaccctggtcaccgtctcctcag

SEQ ID NO: 4 - protein, 112 aa, Canis sp.
EVQLVESGGDLVKPGGSLRLSCVASGFTFSSYHMSWVRQAPGKGLQWVAYINSGGSSTTYADAVKGRFTISRDNAKKTLYLQMNSLRAEDTAVYYCSTFDYWGQGTLVTVSS

SEQ ID NO: 5 - DNA, 319 bp, Canis sp.
tcctatgtgctgacacagctgccatccagaaatgtgaccctgaagcagccggcccacatcacctgtgggggagacaacattggaagtaaaagtgttcactggtaccagcagaagctgggccaggcccctgtactgattatctattatgataccaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 6 - protein, 106 aa, Canis sp.
SYVLTQLPSRNVTLKQPAHITCGGDNIGSKSVHWYQQKLGQAPVLIIYYDTRRPTGIPERFSGANSGNTATLTISGALAEDEADYYCQVWDSSGNVFGGGTHLTVL

SEQ ID NO: 7 - protein, 8 aa, Canis sp.
GFTFSSYH

SEQ ID NO: 8 - protein, 8 aa, Canis sp.
INSGGSST

SEQ ID NO: 9 - protein, 5 aa, Canis sp.
STFDY

SEQ ID NO: 10 - protein, 6 aa, Canis sp.
NIGSKS

SEQ ID NO: 11 - protein, 3 aa, Canis sp.
YDT

SEQ ID NO: 12 - protein, 9 aa, Canis sp.
QVWDSSGNV

SEQ ID NO: 13 - DNA, 337 bp, Canis sp.
gaggtgcagttggtggagtctgggggagacctggtgaagcctggggggtccctgagactttcctgtgtggcctctggaatcatcttcagtaactaccacatgagctgggtccgccaggttccagggaagggacttcagtgggtcgcatacattaacagtggtggaagtactataaactatgcagacggtgtgaagggccgattcaccatctccagagacaacgccaagaactcgctgtatcttcagatgaacagcctgagaggcgaggacacggccgtgtattactgtacgacttatgactcctggggccagggaaccctggtcaccgtctcctcag

SEQ ID NO: 14 - protein, 112 aa, Canis sp.
EVQLVESGGDLVKPGGSLRLSCVASGIIFSNYHMSWVRQVPGKGLQWVAYINSGGSTINYADGVKGRFTISRDNAKNSLYLQMNSLRGEDTAVYYCTTYDSWGQGTLVTVSS

SEQ ID NO: 15 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgttaatgtgttcggcggaggcacccatctgaccgtcctcg

SEQ ID NO: 16 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDSSVNVFGGGTHLTVL

SEQ ID NO: 17 - protein, 8 aa, Canis sp.
GIIFSNYH

SEQ ID NO: 18 - protein, 8 aa, Canis sp.
INSGGSTI

SEQ ID NO: 19 - protein, 5 aa, Canis sp.
TTYDS

SEQ ID NO: 20 - protein, 6 aa, Canis sp.
NIGSKS

SEQ ID NO: 21 - protein, 3 aa, Canis sp.
YDS

SEQ ID NO: 22 - protein, 9 aa, Canis sp.
QVWDSSVNV

SEQ ID NO: 23 - DNA, 337 bp, Canis sp.
gaggtgcagctggtggagtctgggggagacctggtgaagcctggggggtccctgagactttcctgtgtggcctctggaatcaccttcagtaggtaccacatgagctgggtccgccaggctccagggaaggggcttcaatgggtcgcatacattaacagtggtggaagtagcacaagctatccagacgctgtgaagggccgattcaccatctccagagacaacgccaagaacacgctgtatcttcagatggacagcctgagagccgaggacacggccctgtattactgtacgacctttgactactggggccagggaaccctggtcaccgtctcctcag

SEQ ID NO: 24 - protein, 112 aa, Canis sp.
EVQLVESGGDLVKPGGSLRLSCVASGITFSRYHMSWVRQAPGKGLQWVAYINSGGSSTSYPDAVKGRFTISRDNAKNTLYLQMDSLRAEDTALYYCTTFDYWGQGTLVTVSS

SEQ ID NO: 25 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaaactgggccaggcccctgtactgattatctattatgatagcaggaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 26 - protein, 106 aa, Canis sp.
SYVLTQLPSKNVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 27 - protein, 8 aa, Canis sp.
GITFSRYH

SEQ ID NO: 28 - protein, 8 aa, Canis sp.
INSGGSST

SEQ ID NO: 29 - protein, 5 aa, Canis sp.
TTFDY

SEQ ID NO: 30 - protein, 6 aa, Canis sp.
NIGSKS

SEQ ID NO: 31 - protein, 3 aa, Canis sp.
YDS

SEQ ID NO: 32 - protein, 9 aa, Canis sp.
QVWDSSANV

SEQ ID NO: 33 - DNA, 337 bp, Canis sp.
gaagtgcagctggtggagtctgggggagagctggtgaagcctggggggtccctgagagtttcctgtgtggcctctggaatcaccttcagtcgctaccacatgagttgggtccgccaggctccagggaagggacttcagtgggtcgcattcattaacactgatagaagtagcccaaaatatgcagactctgtgaagggccgattcaccatctccagagacaacgccaagaacacgctgtatcttcagatgaacagcctgagagccgaggacacggccatttattactgtacgagttttgactactggggccagggaaccctggtcaccgtctcctcaa

SEQ ID NO: 34 - protein, 112 aa, Canis sp.
EVQLVESGGELVKPGGSLRVSCVASGITFSRYHMSWVRQAPGKGLQWVAFINTDRSSPKYADSVKGRFTISRDNAKNTLYLQMNSLRAEDTAIYYCTSFDYWGQGTLVTVSS

SEQ ID NO: 35 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagtagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 36 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDSSANVFGGGTHLTVL

SEQ ID NO: 37 - protein, 8 aa, Canis sp.
GITFSRYH

SEQ ID NO: 38 - protein, 8 aa, Canis sp.
INTDRSSP

SEQ ID NO: 39 - protein, 5 aa, Canis sp.
TSFDY

SEQ ID NO: 40 - protein, 6 aa, Canis sp.
NIGSKS

SEQ ID NO: 41 - protein, 3 aa, Canis sp.
YDS

SEQ ID NO: 42 - protein, 9 aa, Canis sp.
QVWDSSANV

SEQ ID NO: 43 - DNA, 337 bp, Canis sp.
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtgactcagactatgcagacgctgtgaagggccgattcaccatctccagagacaacgccaagaatacgctgtatcttcagatgaacagcctgagagccgaggacacggccgtgtattattgtacgagtctggactactggggccagggaaccctggtcaccgtctcctcag

SEQ ID NO: 44 - protein, 112 aa, Canis sp.
EVQLVESGGDLVKPGGSLRLSCVASGFTFSRYHMSWVRQAPGKGLQWVAYINSGGSDSDYADAVKGRFTISRDNAKNTLYLQMNSLRAEDTAVYYCTSLDYWGQGTLVTVSS

SEQ ID NO: 45 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgtcaggtgtgggacaggagtggtaatgtgttcggcggaggcacccatctgaccgtcctcg

SEQ ID NO: 46 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDRSGNVFGGGTHLTVL

SEQ ID NO: 47 - protein, 8 aa, Canis sp.
GFTFSRYH

SEQ ID NO: 48 - protein, 8 aa, Canis sp.
INSGGSDS

SEQ ID NO: 49 - protein, 5 aa, Canis sp.
TSLDY

SEQ ID NO: 50 - protein, 6 aa, Canis sp.
NIGSKS

SEQ ID NO: 51 - protein, 3 aa, Canis sp.
YDS

SEQ ID NO: 52 - protein, 9 aa, Canis sp.
QVWDRSGNV

SEQ ID NO: 53 - protein, 106 aa, Canis sp.
SYVLTQLPSKQVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 54 - protein, 106 aa, Canis sp.
SYVLTQLPSKAVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 55 - protein, 106 aa, Canis sp.
SYVLTQLPSKEVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 56 - protein, 106 aa, Canis sp.
SYVLTQLPSKNVALKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 57 - protein, 106 aa, Canis sp.
SYVLTQLPSKSVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 58 - protein, 106 aa, Canis sp.
SYVLTQLPSKTVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 59 - protein, 25 aa, Canis sp.
SYVLTQLPSKNVTLKQPAHITCGGD

SEQ ID NO: 60 - protein, 25 aa, Canis sp.
SYVLTQLPSKQVTLKQPAHITCGGD

SEQ ID NO: 61 - protein, 25 aa, Canis sp.
SYVLTQLPSKAVTLKQPAHITCGGD

SEQ ID NO: 62 - protein, 25 aa, Canis sp.
SYVLTQLPSKEVTLKQPAHITCGGD

SEQ ID NO: 63 - protein, 25 aa, Canis sp.
SYVLTQLPSKNVALKQPAHITCGGD

SEQ ID NO: 64 - protein, 25 aa, Canis sp.
SYVLTQLPSKSVTLKQPAHITCGGD

SEQ ID NO: 65 - protein, 25 aa, Canis sp.
SYVLTQLPSKTVTLKQPAHITCGGD

SEQ ID NO: 66 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 67 - protein, 106 aa, Canis sp.
GQPKASPSVTLFPPSSEELGANKATLVCLISDFYPSGVTVAWKADGSPITQGVETTKPSKQSNNKYAASSYLSLTPDKWKSHSSFSCLVTHEGSTVEKKVAPAECS

SEQ ID NO: 68 - DNA, 337 bp, Canis sp.
gaggtgcagctggtggagagcggcggcgacctggtgaagcctggcggcagcctgagactgagctgcgtggcaagcggcatcaccttcagcagataccacatgagctgggtgagacaagcccctggcaagggcctgcagtgggtggcctacatcaacagcggcggcagcagcacaagctaccctgacgccgtgaagggcagattcaccatcagcagagacaacgccaagaacaccctgtacctgcagatggacagcctgagagccgaggacaccgccctgtactactgcaccaccttcgactactggggccaaggcaccctggtgaccgtgagcagcg

SEQ ID NO: 69 - DNA, 319 bp, Canis sp.
agctacgtgctgacacagctgcctagcaagaacgtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggcggcacccacctgaccgtgctgg

SEQ ID NO: 70 - protein, 19 aa, Canis sp.
MGWSCIILFLVATATGVHS

SEQ ID NO: 71 - protein, 19 aa, Canis sp.
MESALSWVFLVTILKGVQG

SEQ ID NO: 72 - protein, 19 aa, Canis sp.
MAWTHLLLSLLALCTGSVA

SEQ ID NO: 73 - protein, 110 aa, Canis sp.
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 74 - protein, 98 aa, Canis sp.
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCAS

SEQ ID NO: 75 - protein, 15 aa, Canis sp.
NFDYWGQGTLVTVSS

SEQ ID NO: 76 - protein, 96 aa, Canis sp.
SYVLTQLPSKNVTLKQPAHITCGGDNIGSKSVHWYQQKLGQAPVLIIYYDSSRPTGIPERFSGANSGNTATLTISGALAEDEADYYCQVWDSSAKA

SEQ ID NO: 77 - protein, 12 aa, Canis sp.
SVFGGGTHLTVL

SEQ ID NO: 78 - DNA, 394 bp, Canis sp.
atggagtctgcgttcagctgggtgttccttgtcactattttaaaaggtgtccagggtgcggtgcagctggtggagtcagggggagacctggtgaagcctggggggtccctgagactttcctgtgtggcctctggaatcaccttcagtaggtaccacatgagttgggtccgccaggctccagggaaggggcttcagtgggtcgcatacattaatagtggtggaagtgccacaagttatacagacgctgtgaagggccgattcagcatctccagagacaacggcaaaaacacgctttatcttcagatgaatagcctgagagtcgaggactcggccgtctattactgtacgacccttgacctctggggccagggaaccctggtcaccgtctcctcag

SEQ ID NO: 79 - protein, 112 aa, Canis sp.
AVQLVESGGDLVKPGGSLRLSCVASGITFSRYHMSWVRQAPGKGLQWVAYINSGGSATSYTDAVKGRFSISRDNGKNTLYLQMNSLRVEDSAVYYCTTLDLWGQGTLVTVSS

SEQ ID NO: 80 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgtcagatgtgggacagcagtgctaatgtgttcggcggaggcacccatctgaccgtcctcg

SEQ ID NO: 81 - protein, 106 aa, Canis sp.
SYVLTQLPSKNVTLKQPAHITCGGDNIGSKSVHWYQQKLGQAPVLIIYYDSRRPTGIPERFSGANSGNTATLTINGALAEDEADYYCQMWDSSANVFGGGTHLTVL

SEQ ID NO: 82 - protein, 8 aa, Canis sp.
GITFSRYH

SEQ ID NO: 83 - protein, 8 aa, Canis sp.
INSGGSAT

SEQ ID NO: 84 - protein, 5 aa, Canis sp.
TTLDL

SEQ ID NO: 85 - protein, 6 aa, Canis sp.
NIGSKS

SEQ ID NO: 86 - protein, 3 aa, Canis sp.
YDS

SEQ ID NO: 87 - protein, 9 aa, Canis sp.
QMWDSSANV

SEQ ID NO: 88 - DNA, 394 bp, Canis sp.
atggagtctgcgctcagctgggtgttccttgtcactattttaaaaggtgtccagggtgagatacaactggtggagtctgggggagacctggtgaagcctggggggtccctgagactttcctgtgtggcctctggattcaccttcagtcgctaccacatgagctgggtccgccaggctccagggaaggggcttcagtgggtcgcatatattaacactggtggaagtaacacaaactatgcagacgctgtgaagggccgattcaccatctccagagacaacgccaagaacacgctgtatcttcagatgaacagcctgagagccgaggacacggccgtgtattactgtacgacccttgactcctggggccagggaaccctggtcaccgtctcctcag

SEQ ID NO: 89 - protein, 112 aa, Canis sp.
EIQLVESGGDLVKPGGSLRLSCVASGFTFSRYHMSWVRQAPGKGLQWVAYINTGGSNTNYADAVKGRFTISRDNAKNTLYLQMNSLRAEDTAVYYCTTLDSWGQGTLVTVSS

SEQ ID NO: 90 - DNA, 376 bp, Canis sp.
atggcctggacccacctcctcctgagcctcctggctctctgcacaggttctgtggcctcctatgtgctgacacagctgccattcaaaaatgtgaccctgaagcagccggcccacatcacctgtgggggagacaacattggaagtaaaagtgttcactggtaccagcagaagctgggccaggcccctgtattgattatctattatgatagtagaaggccgacagggatccctgagcgattctccggcgccaactcggggaacacggccaccctgaccatcagcggggccctggccgaggacgaggctgactattactgccaggtgtgggacagtagtgttaatgtgttcggcggaggcacccatctgaccgtcctcg

SEQ ID NO: 91 - protein, 106 aa, Canis sp.
SYVLTQLPFKNVTLKQPAHITCGGDNIGSKSVHWYQQKLGQAPVLIIYYDSRRPTGIPERFSGANSGNTATLTISGALAEDEADYYCQVWDSSVNVFGGGTHLTVL

SEQ ID NO: 92 - protein, 8 aa, Canis sp.
GFTFSRYH

SEQ ID NO: 93 - protein, 8 aa, Canis sp.
INTGGSNT

SEQ ID NO: 94 - protein, 5 aa, Canis sp.
TTLDS

SEQ ID NO: 95 - protein, 6 aa, Canis sp.
NIGSKS

SEQ ID NO: 96 - protein, 3 aa, Canis sp.
YDS

SEQ ID NO: 97 - protein, 9 aa, Canis sp.
QVWDSSVNV

SEQ ID NO: 98 - DNA, 394 bp, Canis sp.
atggagtctgcgctcagctgggtgttccttgtcactattttaaaaggtgtccagggtgaactacagctggtggagtctgggggagacctggtgaagcctggggggtccctgagactttcctgtgtggcctccggattcaccttcagtagatactacttgaactgggtccgccaggctccagggaaggggcttcagtgggtcgcattcattaccagtggtggcagtagcacaaactatgcagacgctgtgaagggccgattcaccatctccagagacaacgccaagaacacactgtatcttcagatgaatagcctgagagccgaggacacggccgtttattactgtacgagcttcgactcctggggccagggaaccctggtcaccgtctcctcag

SEQ ID NO: 99 - protein, 112 aa, Canis sp.
ELQLVESGGDLVKPGGSLRLSCVASGFTFSRYYLNWVRQAPGKGLQWVAFITSGGSSTNYADAVKGRFTISRDNAKNTLYLQMNSLRAEDTAVYYCTSFDSWGQGTLVTVSS

SEQ ID NO: 100 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactaattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 101 - protein, 106 aa, Canis sp.; identical to SEQ ID NO: 36

SEQ ID NO: 102 - protein, 8 aa, Canis sp.
GFTFSRYY

SEQ ID NO: 103 - protein, 8 aa, Canis sp.
ITSGGSST

SEQ ID NO: 104 - protein, 5 aa, Canis sp.
TSFDS

SEQ ID NO: 105 - protein, 6 aa, Canis sp.
NIGSKS

SEQ ID NO: 106 - protein, 3 aa, Canis sp.
YDS

SEQ ID NO: 107 - protein, 9 aa, Canis sp.
QVWDSSANV

SEQ ID NO: 108 - DNA, 394 bp, Canis sp.
atggagtctgcgctcagctgggtgttccttgtcactattttaaaaggtgtccagggtgagatgcagctggtggaatctgggggagccctggtgaagcctggggggtccctgagactctcctgtgtggcctctggattcacctttaggagataccacatgagttgggtccgccaggctccaggacaggggcttcagtgggtcgcatacattgacagtgatggaagtcctacaagttatggagactctgtgaagggccgattcaccatctccagagacaacgccaagaatacgctatttcttcagatgaacagtctgagagtcgacgacacggccgtatattactgtacgactcttgactactggggccggggaaccctggtcaccgtctcctcag

SEQ ID NO: 109 - protein, 112 aa, Canis sp.
EMQLVESGGALVKPGGSLRLSCVASGFTFRRYHMSWVRQAPGQGLQWVAYIDSDGSPTSYGDSVKGRFTISRDNAKNTLFLQMNSLRVDDTAVYYCTTLDYWGRGTLVTVSS

SEQ ID NO: 110 - DNA, 376 bp, Canis sp.
atggcctggacccacctcctcctgagcctcctggctctctgcacaggttctgtggcctcctatgtgctgacacaggtgccatccaaaaatgtgaccctgaagcagccggcccacatcacctgtgggggagacaacattggaggtaaaagtgttcactggtaccagcagaagctgggccaggcccctctactgattatctattatgataccaggaggccgacagggatccctgagcgcttctccggcgccaactcggggaacacggccaccctgaccatcagcagggccctggccgaggacgaggctgactattactgccaggtgtgggacagcagtggtaatgtgttcggcggaggcacccatctgaccgtcatcg

SEQ ID NO: 111 - protein, 106 aa, Canis sp.
SYVLTQVPSKNVTLKQPAHITCGGDNIGGKSVHWYQQKLGQAPLLIIYYDTRRPTGIPERFSGANSGNTATLTISRALAEDEADYYCQVWDSSGNVFGGGTHLTVI

SEQ ID NO: 112 - protein, 8 aa, Canis sp.
GFTFRRYH

SEQ ID NO: 113 - protein, 8 aa, Canis sp.
IDSDGSPT

SEQ ID NO: 114 - protein, 5 aa, Canis sp.
TTLDY

SEQ ID NO: 115 - protein, 6 aa, Canis sp.
NIGGKS

SEQ ID NO: 116 - protein, 3 aa, Canis sp.
YDT

SEQ ID NO: 117 - protein, 9 aa, Canis sp.
QVWDSSGNV

SEQ ID NO: 118 - DNA, 394 bp, Canis sp.
atggagtctgcgctcagctgggtgttccttgtcactattttaaaaggtgtccagggtgagatgcaactggtggagtctgggggagacctggtgaagcctggggggtccctgagactttcctgtgtggcctctggattcaccttcagtcgctaccacatgagctgggtccgccaggctccagggaaggggcttcagtgggtcgcatacattaatagtggtggaagtcccacaaactatgcagacgctgtgaagggccgattcaccatctccagagacaacgccaagaacacactatatcttcagatgaacagcctgagagccgaggacacggccgtgtattactgtacgacctacgaccagtggggccagggaaccctggtcaccgtctcctcag

SEQ ID NO: 119 - protein, 112 aa, Canis sp.
EMQLVESGGDLVKPGGSLRLSCVASGFTFSRYHMSWVRQAPGKGLQWVAYINSGGSPTNYADAVKGRFTISRDNAKNTLYLQMNSLRAEDTAVYYCTTYDQWGQGTLVTVSS

SEQ ID NO: 120 - DNA, 376 bp, Canis sp.
atggcctggacccacctcctcctgagcctcctggctctctgcacaggttctgtggcctcctatgtgctgacacagctgccatccaaaagtgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacaccagtgctaatgtgttcggcggaggcacccatctgaccgtcctcg

SEQ ID NO: 121 - protein, 106 aa, Canis sp.
SYVLTQLPSKSVTLKQPAHITCGGDNIGSKSVHWYQQKLGQAPVLIIYYDSSRPTGIPERFSGANSGNTATLTISGALAEDEADYYCQVWDTSANVFGGGTHLTVL

SEQ ID NO: 122 - protein, 8 aa, Canis sp.
GFTFSRYH

SEQ ID NO: 123 - protein, 8 aa, Canis sp.
INSGGSPT

SEQ ID NO: 124 - protein, 5 aa, Canis sp.
TTYDQ

SEQ ID NO: 125 - protein, 6 aa, Canis sp.
NIGSKS

SEQ ID NO: 126 - protein, 3 aa, Canis sp.
YDS

SEQ ID NO: 127 - protein, 9 aa, Canis sp.
QVWDTSANV

SEQ ID NO: 128 - DNA, 394 bp, Canis sp.
atggagtctgcgctcagctgggtgttccttgtcactattttaaaaggtgtccagggtgaggtgcacctggtggagtctgggggagacctggtgaagcctggggggtccctgagactttcctgtgtggcctctggaatcaccttcaatagatattatatgaactgggtccgccagactccagggaaggggcttcagtgggtcgcatacatagacagtggtggaagtcccacaacctatgcagacgctgtaaagggccgattcaccatctccagagacaacgccaagaacacgttgtatcttcagatgaacagcctgagagccgaggacacggccgtatattactgtacgagttttgactactggggccagggaaccctggtcaccgtctcctcag

SEQ ID NO: 129 - protein, 112 aa, Canis sp.
EVHLVESGGDLVKPGGSLRLSCVASGITFNRYYMNWVRQTPGKGLQWVAYIDSGGSPTTYADAVKGRFTISRDNAKNTLYLQMNSLRAEDTAVYYCTSFDYWGQGTLVTVSS

SEQ ID NO: 130 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgataccagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 131 - protein, 106 aa, Canis sp.
SYVLTQLPSKNVTLKQPAHITCGGDNIGSKSVHWYQQKLGQAPILIIYYDTSRPTGIPERFSGANSGNTATLTISGALAEDEADYYCQVWDSSANVFGGGTHLTVL

SEQ ID NO: 132 - protein, 8 aa, Canis sp.
GITFNRYY

SEQ ID NO: 133 - protein, 8 aa, Canis sp.
IDSGGSPT

SEQ ID NO: 134 - protein, 5 aa, Canis sp.
TSFDY

SEQ ID NO: 135 - protein, 6 aa, Canis sp.
NIGSKS

SEQ ID NO: 136 - protein, 3 aa, Canis sp.
YDT

SEQ ID NO: 137 - protein, 9 aa, Canis sp.
QVWDSSANV

SEQ ID NO: 138 - DNA, 394 bp, Canis sp.
atggagtctgcgctcagctgggtgttccttgtcactattttaaaaggtgtccagggtgaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcattcattaacactggtagaagtagcacaaactatgcagacgctgtggcgggccgattcaccatctccagagacaacgccaagaacacgctgtatcttcagatgaacagcctgagagccgaggacacggccgtgtattactgttcgaccctcgactactggggccagggaaccctggtcaccgtctcctcag

SEQ ID NO: 139 - protein, 112 aa, Canis sp.
EVQLVESGGDLVKPGGSLRLSCVASGFTFSRYHMSWVRQAPGKGLQWVAFINTGRSSTNYADAVAGRFTISRDNAKNTLYLQMNSLRAEDTAVYYCSTLDYWGQGTLVTVSS

SEQ ID NO: 140 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 141 - protein, 106 aa, Canis sp.; identical to SEQ ID NO: 36

SEQ ID NO: 142 - protein, 8 aa, Canis sp.
GFTFSRYH

SEQ ID NO: 143 - protein, 8 aa, Canis sp.
INTGRSST

SEQ ID NO: 144 - protein, 5 aa, Canis sp.
STLDY

SEQ ID NO: 145 - protein, 6 aa, Canis sp.
NIGSKS

SEQ ID NO: 146 - protein, 3 aa, Canis sp.
YDS

SEQ ID NO: 147 - protein, 9 aa, Canis sp.
QVWDSSANV

SEQ ID NO: 148 - DNA, 394 bp, Canis sp.
atggagtctgcgctcagctgggtgttccttgtcactattttaaaaggtgtccagggtgaagtacagctggtggagtctgggggagacctggtgaagcctggggggtccctgagactttcctgtgtggcctccggattcaccttcagtagatactacatgaactgggtccgccaggctccagggaaggggcttcagtgggtcgcattcattaccagtggtggaagtagcacaagctatggagacgctgtgaagggccgattcaccatctccagagacaacgccaagaacacgctgtatcttcagatgaacagcctgagagccgaggacacggccgtgtattactgtacgagcttcgactactggggccagggaaccctggtcaccgtctcctcag

SEQ ID NO: 149 - protein, 112 aa, Canis sp.
EVQLVESGGDLVKPGGSLRLSCVASGFTFSRYYMNWVRQAPGKGLQWVAFITSGGSSTSYGDAVKGRFTISRDNAKNTLYLQMNSLRAEDTAVYYCTSFDYWGQGTLVTVSS

SEQ ID NO: 150 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgctagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 151 - protein, 106 aa, Canis sp.
SYVLTQLPSKNVTLKQPAHITCGGDNIGSKSVHWYQQKLGQAPVLIIYYASSRPTGIPERFSGANSGNTATLTISGALAEDEADYYCQVWDSSANVFGGGTHLTVL

SEQ ID NO: 152 - protein, 8 aa, Canis sp.
GFTFSRYY

SEQ ID NO: 153 - protein, 8 aa, Canis sp.
ITSGGSST

SEQ ID NO: 154 - protein, 5 aa, Canis sp.
TSFDY

SEQ ID NO: 155 - protein, 6 aa, Canis sp.
NIGSKS

SEQ ID NO: 156 - protein, 3 aa, Canis sp.
YAS

SEQ ID NO: 157 - protein, 9 aa, Canis sp.
QVWDSSANV

SEQ ID NO: 158 - DNA, 394 bp, Canis sp.
atggagtctgcgctcagctgggtgttccttgtcattattttaaaaggtgtccagggcgaggtgcagttggtggagtctgggggagacctggtgaagcctggggggtccctgagactttcctgtgtggcctctggattcaccttcagtaggtattacatgacctgggtccgccaggctccagggaaggggcttcagtgggtcgcatacattaacagtgatagaagaagcacaacctatgcagacgctgtgaagggccgattcaccatctccagagacaacgccaaaaacacgctgtatcttcagatgaacagcctgagagccgaggacacggccgtatattactgtacgacccttgaatattggggccagggaaccctggtcaccgtctcctcag

SEQ ID NO: 159 - protein, 112 aa, Canis sp.
EVQLVESGGDLVKPGGSLRLSCVASGFTFSRYYMTWVRQAPGKGLQWVAYINSDRRSTTYADAVKGRFTISRDNAKNTLYLQMNSLRAEDTAVYYCTTLEYWGQGTLVTVSS

SEQ ID NO: 160 - DNA, 376 bp, Canis sp.
atggcctggacccacctcctcctgagcctcctggctctctgcacaggctctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgccaggtgtgggacagcagtgctaatgtgttcggcggaggcacccatctgaccgtcctcg

SEQ ID NO: 161 - protein, 106 aa, Canis sp.
SYVLTQLPSKNVTLKQPAHITCGGDNIGSKSVHWYQQKLGQAPVLIIYYDSRRPTGIPERFSGANSGNTATLTINGALAEDEADYYCQVWDSSANVFGGGTHLTVL

SEQ ID NO: 162 - protein, 8 aa, Canis sp.
GFTFSRYY

SEQ ID NO: 163 - protein, 8 aa, Canis sp.
INSDRRST

SEQ ID NO: 164 - protein, 5 aa, Canis sp.
TTLEY

SEQ ID NO: 165 - protein, 6 aa, Canis sp.
NIGSKS

SEQ ID NO: 166 - protein, 3 aa, Canis sp.
YDS

SEQ ID NO: 167 - protein, 9 aa, Canis sp.
QVWDSSANV

SEQ ID NO: 168 - DNA, 394 bp, Canis sp.
atggagtctgcgctcagctgggtgttccttgtcactattttaaaaggtgtccagggtgaggtgcagttggtggagtctgggggagacctggtgaagccgggggggtccctgagactttcctgtgtggcctccggaatcaccttcagtagatatcacatgagctgggtccgccaggctccggggaaggggcttcagtgggtcgcattcattaacactggtagaagtagcacaaactatgcggacgctgtggcgggccgattcaccatctccagagacaacgccaagaacacgctgtatcttcaaatgaacggcctgacagtcgaggacacggccgtgtattactgttcgaccctcgactactggggccagggaaccctggtcaccgtctcctcag

SEQ ID NO: 169 - protein, 112 aa, Canis sp.
EVQLVESGGDLVKPGGSLRLSCVASGITFSRYHMSWVRQAPGKGLQWVAFINTGRSSTNYADAVAGRFTISRDNAKNTLYLQMNGLTVEDTAVYYCSTLDYWGQGTLVTVSS

SEQ ID NO: 170 - DNA, 376 bp, Canis sp.
atggcctggacccacctcctcctgagcctcctggctctctgcacagattctgtggcctcctatgtgctgacacagctgccatccaaaagtgtgaccctgaagcagccggcccacatcacctgtgggggagacaacattggaagtaaaagtgttcactggtaccagcagaagctgggccaggcccctatactgattatctattatgataacaacaggccgacagggatccctgagcgattctccggcgccaactcggggaacacggccaccctgaccatcagcggggccctggccgaggacgaggctgactattactgccacgtgtgggacagcagtgttaatgtgttcggcggaggcacccatctgaccgtcctcg

SEQ ID NO: 171 - protein, 106 aa, Canis sp.
SYVLTQLPSKSVTLKQPAHITCGGDNIGSKSVHWYQQKLGQAPILIIYYDNNRPTGIPERFSGANSGNTATLTISGALAEDEADYYCHVWDSSVNVFGGGTHLTVL

SEQ ID NO: 172 - protein, 8 aa, Canis sp.
GITFSRYH

SEQ ID NO: 173 - protein, 8 aa, Canis sp.
INTGRSST

SEQ ID NO: 174 - protein, 5 aa, Canis sp.
STLDY

SEQ ID NO: 175 - protein, 6 aa, Canis sp.
NIGSKS

SEQ ID NO: 176 - protein, 3 aa, Canis sp.
YDN

SEQ ID NO: 177 - protein, 9 aa, Canis sp.
HVWDSSVNV

SEQ ID NO: 178 - DNA, 394 bp, Canis sp.
atggagtctgcgctcagctgggtgttccttgtcactattttaaaaggtgtccagggtgaagttcaactggtggagtctgggggagacctggtgaagcctggggggtccctgagactttcctgtgtggcctctggaatcaccttcagtaggtactacatgaactgggtccgccaggctccagggaaggggcttcagtgggtcgcatacattgacagtggtggaagtcccacaacctatgcagacgctgtgaagggccgattcaccatctccagagacaacgccaagaaagcgctgtatcttcagatgaacagcctgagagccgaggacacggccgtatattactgtacgagttttgactactggggccagggaaccctggtcaccgtctcctcag

SEQ ID NO: 179 - protein, 112 aa, Canis sp.
EVQLVESGGDLVKPGGSLRLSCVASGITFSRYYMNWVRQAPGKGLQWVAYIDSGGSPTTYADAVKGRFTISRDNAKKALYLQMNSLRAEDTAVYYCTSFDYWGQGTLVTVSS

SEQ ID NO: 180 - DNA, 376 bp, Canis sp.
atggcctggacccacctcctcctgagcctcctggctctctgcacaggttctgtggcctcctatgtcctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactatttctgccaggtgtgggacagcagtgctaatgtgttcggcggaggcacccatctgaccgtcctcg

SEQ ID NO: 181 - protein, 106 aa, Canis sp.
SYVLTQLPSKNVTLKQPAHITCGGDNIGSKSVHWYQQKLGQAPVLIIYYDSSRPTGIPERFSGANSGNTATLTISGALAEDEADYFCQVWDSSANVFGGGTHLTVL

SEQ ID NO: 182 - protein, 8 aa, Canis sp.
GITFSRYY

SEQ ID NO: 183 - protein, 8 aa, Canis sp.
IDSGGSPT

SEQ ID NO: 184 - protein, 5 aa, Canis sp.
TSFDY

SEQ ID NO: 185 - protein, 6 aa, Canis sp.
NIGSKS

SEQ ID NO: 186 - protein, 3 aa, Canis sp.
YDS

SEQ ID NO: 187 - protein, 9 aa, Canis sp.
QVWDSSANV

SEQ ID NO: 188 - DNA, 394 bp, Canis sp.
atggagtctgcgctcagctgggtgttccttgtcactattttaaaaggtgtccagggtgaggtgcagctggtggagtctgggggagacctggtgaagcctggggggtccctgagactttcctgtgtggcctctggaatcaccttcagtagatattatatgaactgggtccgccaggctccagggaaggggcttcagtgggtcgcatacataaacagtggtggaagtcccacaacctatgcagacgctgtaaagggccgattcaccatctccagagacaacgccaagaacacgttgtatcttcagatgaacagcctgagagccgaggacacggccgtgtattactgtacgagttttgactactggggccagggaaccctggtcaccgtctcctcag

SEQ ID NO: 189 - protein, 112 aa, Canis sp.
EVQLVESGGDLVKPGGSLRLSCVASGITFSRYYMNWVRQAPGKGLQWVAYINSGGSPTTYADAVKGRFTISRDNAKNTLYLQMNSLRAEDTAVYYCTSFDYWGQGTLVTVSS

SEQ ID NO: 190 - DNA, 376 bp, Canis sp.; identical to SEQ ID NO: 130

SEQ ID NO: 191 - protein, 106 aa, Canis sp.; identical to SEQ ID NO: 131

SEQ ID NO: 192 - protein, 8 aa, Canis sp.
GITFSRYY

SEQ ID NO: 193 - protein, 8 aa, Canis sp.
INSGGSPT

SEQ ID NO: 194 - protein, 5 aa, Canis sp.
TSFDY

SEQ ID NO: 195 - protein, 6 aa, Canis sp.
NIGSKS

SEQ ID NO: 196 - protein, 3 aa, Canis sp.
YDT

SEQ ID NO: 197 - protein, 9 aa, Canis sp.
QVWDSSANV

SEQ ID NO: 198 - DNA, 394 bp, Canis sp.
atggagtctgcgctcagctgggtgttccttgtcacaattttaaaaggtgtccagggtgaggtgcaactggtggagtctgggggagacctggtgaagcctggggggtccctgagactttcctgtgtggcctctggactcaccttcagtaggtaccacatgagctggatccgccaggctccagggaaggggcttcagtgggtcgcatacattagcagtggtggaagtagcacaaactatgcaggctctgtgaagggccgattcaccgtctccagagacaacgccaagaacactctgtatcttcagatgaacagtctgagagccgaagacacggccgtctattactgtacgagtcttgactactggggccagggaaccctggtcaccgtctcctcag

SEQ ID NO: 199 - protein, 112 aa, Canis sp.
EVQLVESGGDLVKPGGSLRLSCVASGLTFSRYHMSWIRQAPGKGLQWVAYISSGGSSTNYAGSVKGRFTVSRDNAKNTLYLQMNSLRAEDTAVYYCTSLDYWGQGTLVTVSS

SEQ ID NO: 200 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagtaggaggccgacagggatccctgagcgattctccggcgccaactcggggaacacggccaccctgaccatcagcggggccctggccgaggacgaggctgactattactgccaggtgtgggacagcagtgataatgtgttcggcggaggcacccatctgaccgtcctcg

SEQ ID NO: 201 - protein, 106 aa, Canis sp.
SYVLTQLPSKNVTLKQPAHITCGGDNIGSKSVHWYQQKLGQAPVLIIYYDSRRPTGIPERFSGANSGNTATLTISGALAEDEADYYCQVWDSSDNVFGGGTHLTVL

SEQ ID NO: 202 - protein, 8 aa, Canis sp.
GLTFSRYH

SEQ ID NO: 203 - protein, 8 aa, Canis sp.
ISSGGSST

SEQ ID NO: 204 - protein, 5 aa, Canis sp.
TSLDY

SEQ ID NO: 205 - protein, 6 aa, Canis sp.
NIGSKS

SEQ ID NO: 206 - protein, 3 aa, Canis sp.
YDS

SEQ ID NO: 207 - protein, 9 aa, Canis sp.
QVWDSSDNV

SEQ ID NO: 208 - DNA, 394 bp, Canis sp.
atggagtctgcgctcagctgggtgttccttgtcactattttaaaaggtgtccagggtgaggtgcagctggtggagtctgggggagacctggtgaagccgggggggtccctgagactttcctgtgtggcctctggactcaccttcagtagataccacatgagttgggtccgccaggctccagggaaggggcttcagtgggtcgcattcattaacagtggtagaagtgacacaacctatgcagacgctgtgaagggccgattcaccatctccagagacaacgccaagaacacgctgtatcttcagatgaacagcctgagagccgaggacacggccgtttattactgtacgacttttgactactggggccagggaaccctggtcaccgtctcctcag

SEQ ID NO: 209 - protein, 112 aa, Canis sp.
EVQLVESGGDLVKPGGSLRLSCVASGLTFSRYHMSWVRQAPGKGLQWVAFINSGRSDTTYADAVKGRFTISRDNAKNTLYLQMNSLRAEDTAVYYCTTFDYWGQGTLVTVSS

SEQ ID NO: 210 - DNA, 376 bp, Canis sp.
atggcctggacccacctcctcctgagcctcctggctctctgcacaggttatgtggcctcctatgtgctgacacagctgccatccaaaaatgtgaccctgaagcagccggcccacatcacctgtgggggagacaacattggaagtaaaagtgttcactggtaccagcagaagctgggccaggcccctgcactgattatctattatgatagcagcaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 211 - protein, 106 aa, Canis sp.
SYVLTQLPSKNVTLKQPAHITCGGDNIGSKSVHWYQQKLGQAPALIIYYDSSRPTGIPERFSGANSGNTATLTISGALAEDEADYYCQVWDSSANVFGGGTHLTVL

SEQ ID NO: 212 - protein, 8 aa, Canis sp.
GLTFSRYH

SEQ ID NO: 213 - protein, 8 aa, Canis sp.
INSGRSDT

SEQ ID NO: 214 - protein, 5 aa, Canis sp.
TTFDY

SEQ ID NO: 215 - protein, 6 aa, Canis sp.
NIGSKS

SEQ ID NO: 216 - protein, 3 aa, Canis sp.
YDS

SEQ ID NO: 217 - protein, 9 aa, Canis sp.
QVWDSSANV

SEQ ID NO: 218 - DNA, 394 bp, Canis sp.
atggagtctgcgctcagctgggtgttcctcgtcactattttaaaaggtgtccagggtgaggtgcagttggtggagtctgggggagacctggtgaagcctggggggtccctgagactttcctgtgtggcctctggattaaccttcagtagatattacatgaactgggtccgccaggctccagggaaggggcttcagtgggtcgcatatattgatagtggtggaagtcccacaaactatgcagacgctgtgaagggccgattcaccatctccagagacaacgccaagaacacactgtatcttcagatgaacagcctgagagccgaggacacggccgtatattattgttcgagttttgactactggggccagggaaccctggtcaccgtctcctcag

SEQ ID NO: 219 - protein, 112 aa, Canis sp.
EVQLVESGGDLVKPGGSLRLSCVASGLTFSRYYMNWVRQAPGKGLQWVAYIDSGGSPTNYADAVKGRFTISRDNAKNTLYLQMNSLRAEDTAVYYCSSFDYWGQGTLVTVSS

SEQ ID NO: 220 - DNA, 376 bp, Canis sp.
atggcctggacccacctcctcctgagcctcctggctctctgcacaggttctgtggcctcctatgtggtgacacagctgccatccaatactgtgaccctgaagcagccggcccacatcacctgtgggggagacaacattggaagtaaaagtgttcactggtaccagcagaagctgggccaggcccctgtgctgattatctattatgatagcaggaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 221 - protein, 106 aa, Canis sp.
SYVVTQLPSNTVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 222 - protein, 8 aa, Canis sp.
GLTFSRYY

SEQ ID NO: 223 - protein, 8 aa, Canis sp.
IDSGGSPT

SEQ ID NO: 224 - protein, 5 aa, Canis sp.
SSFDY

SEQ ID NO: 225 - protein, 6 aa, Canis sp.
NIGSKS

SEQ ID NO: 226 - protein, 3 aa, Canis sp.
YDS

SEQ ID NO: 227 - protein, 9 aa, Canis sp.
QVWDSSANV

SEQ ID NO: 228 - DNA, 394 bp, Canis sp.
atggagtctgcgctcagctgggtgttccttgtcactattttaaaaggtgtccagggtgaggtgcagctggtgcagtctgggggagacctggtgaagcctggggggtccctgagactttcctgtgtggcctctggatacaccttcactaggtatcacatgagttgggtccgccaggctccagggaaggggcttcagtgggtcgcattcattaatagtgatggaactggtataacctatggagacgctgtgaagggccgattcaccatctccagagacaatgccaagaacacgctgtatcttcagatgaacagcctgagagccgaggacacggccatctattactgtacgacttttgactcctggggccagggaaccctggtcaccgtctcctcag

SEQ ID NO: 229 - protein, 112 aa, Canis sp.
EVQLVQSGGDLVKPGGSLRLSCVASGYTFTRYHMSWVRQAPGKGLQWVAFINSDGTGITYGDAVKGRFTISRDNAKNTLYLQMNSLRAEDTAIYYCTTFDSWGQGTLVTVSS

SEQ ID NO: 230 - DNA, 376 bp, Canis sp.
atggcctggacccacctcctcctgagcctcctggctctctgcacaggttctgtggcctcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagcaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 231 - protein, 106 aa, Canis sp.
SYVLTQLPSKNVTLKQPAHITCGGDNIGSKSVHWYQQKLGQAPILIIYYDSRRPTGIPERFSGANSGNTATLTISGALAEDEADYYCQVWDSSGNVFGGGTHLTVL

SEQ ID NO: 232 - protein, 8 aa, Canis sp.
GYTFTRYH

SEQ ID NO: 233 - protein, 8 aa, Canis sp.
INSDGTGI

SEQ ID NO: 234 - protein, 5 aa, Canis sp.
TTFDS

SEQ ID NO: 235 - protein, 6 aa, Canis sp.
NIGSKS

SEQ ID NO: 236 - protein, 3 aa, Canis sp.
YDS

SEQ ID NO: 237 - protein, 9 aa, Canis sp.
QVWDSSGNV

SEQ ID NO: 238 - protein, 106 aa, Canis sp.
SYVLTQLPSKSVTLKQPAHITCGGDNIGSKSVHWYQQKLGQAPVLIIYYDSRRPTGIPERFSGANSGNTATLTINGALAEDEADYYCQMWDSSANVFGGGTHLTVL

SEQ ID NO: 239 - protein, 106 aa, Canis sp.
SYVLTQLPFKSVTLKQPAHITCGGDNIGSKSVHWYQQKLGQAPVLIIYYDSRRPTGIPERFSGANSGNTATLTISGALAEDEADYYCQVWDSSVNVFGGGTHLTVL

SEQ ID NO: 240 - protein, 106 aa, Canis sp.
SYVLTQLPSKSVTLKQPAHITCGGDNIGSKSVHWYQQKLGQAPVLIIYYDSSRPTGIPERFSGANSGNTATLTISGALAEDEADYYCQVWDSSANVFGGGTHLTVL

SEQ ID NO: 241 - protein, 106 aa, Canis sp.
SYVLTQVPSKSVTLKQPAHITCGGDNIGGKSVHWYQQKLGQAPLLIIYYDTRRPTGIPERFSGANSGNTATLTISRALAEDEADYYCQVWDSSGNVFGGGTHLTVI

SEQ ID NO: 242 - protein, 106 aa, Canis sp.
SYVLTQLPSKSVTLKQPAHITCGGDNIGSKSVHWYQQKLGQAPILIIYYDTSRPTGIPERFSGANSGNTATLTISGALAEDEADYYCQVWDSSANVFGGGTHLTVL

SEQ ID NO: 243 - protein, 106 aa, Canis sp.; identical to SEQ ID NO: 240

SEQ ID NO: 244 - protein, 106 aa, Canis sp.
SYVLTQLPSKSVTLKQPAHITCGGDNIGSKSVHWYQQKLGQAPVLIIYYASSRPTGIPERFSGANSGNTATLTISGALAEDEADYYCQVWDSSANVFGGGTHLTVL

SEQ ID NO: 245 - protein, 106 aa, Canis sp.
SYVLTQLPSKSVTLKQPAHITCGGDNIGSKSVHWYQQKLGQAPVLIIYYDSRRPTGIPERFSGANSGNTATLTINGALAEDEADYYCQVWDSSANVFGGGTHLTVL

SEQ ID NO: 246 - protein, 106 aa, Canis sp.
SYVLTQLPSKSVTLKQPAHITCGGDNIGSKSVHWYQQKLGQAPVLIIYYDSSRPTGIPERFSGANSGNTATLTISGALAEDEADYFCQVWDSSANVFGGGTHLTVL

SEQ ID NO: 247 - protein, 106 aa, Canis sp.; identical to SEQ ID NO: 242

SEQ ID NO: 248 - protein, 106 aa, Canis sp.
SYVLTQLPSKSVTLKQPAHITCGGDNIGSKSVHWYQQKLGQAPVLIIYYDSRRPTGIPERFSGANSGNTATLTISGALAEDEADYYCQVWDSSDNVFGGGTHLTVL

SEQ ID NO: 249 - protein, 106 aa, Canis sp.
SYVLTQLPSKSVTLKQPAHITCGGDNIGSKSVHWYQQKLGQAPALIIYYDSSRPTGIPERFSGANSGNTATLTISGALAEDEADYYCQVWDSSANVFGGGTHLTVL

SEQ ID NO: 250 - protein, 106 aa, Canis sp.
SYVLTQLPSKSVTLKQPAHITCGGDNIGSKSVHWYQQKLGQAPILIIYYDSRRPTGIPERFSGANSGNTATLTISGALAEDEADYYCQVWDSSGNVFGGGTHLTVL

SEQ ID NO: 251 - DNA, 337 bp, Canis sp.
gcggtgcagctggtggagtcagggggagacctggtgaagcctggggggtccctgagactttcctgtgtggcctctggaatcaccttcagtaggtaccacatgagttgggtccgccaggctccagggaaggggcttcagtgggtcgcatacattaatagtggtggaagtgccacaagttatacagacgctgtgaagggccgattcagcatctccagagacaacggcaaaaacacgctttatcttcagatgaatagcctgagagtcgaggactcggccgtctattactgtacgacccttgacctctggggccagggaaccctggtcaccgtctcctcag

SEQ ID NO: 252 - DNA, 337 bp, Canis sp.
gagatacaactggtggagtctgggggagacctggtgaagcctggggggtccctgagactttcctgtgtggcctctggattcaccttcagtcgctaccacatgagctgggtccgccaggctccagggaaggggcttcagtgggtcgcatatattaacactggtggaagtaacacaaactatgcagacgctgtgaagggccgattcaccatctccagagacaacgccaagaacacgctgtatcttcagatgaacagcctgagagccgaggacacggccgtgtattactgtacgacccttgactcctggggccagggaaccctggtcaccgtctcctcag

SEQ ID NO: 253 - DNA, 337 bp, Canis sp.
gaactacagctggtggagtctgggggagacctggtgaagcctggggggtccctgagactttcctgtgtggcctccggattcaccttcagtagatactacttgaactgggtccgccaggctccagggaaggggcttcagtgggtcgcattcattaccagtggtggcagtagcacaaactatgcagacgctgtgaagggccgattcaccatctccagagacaacgccaagaacacactgtatcttcagatgaatagcctgagagccgaggacacggccgtttattactgtacgagcttcgactcctggggccagggaaccctggtcaccgtctcctcag

SEQ ID NO: 254 - DNA, 337 bp, Canis sp.
gagatgcagctggtggaatctgggggagccctggtgaagcctggggggtccctgagactctcctgtgtggcctctggattcacctttaggagataccacatgagttgggtccgccaggctccaggacaggggcttcagtgggtcgcatacattgacagtgatggaagtcctacaagttatggagactctgtgaagggccgattcaccatctccagagacaacgccaagaatacgctatttcttcagatgaacagtctgagagtcgacgacacggccgtatattactgtacgactcttgactactggggccggggaaccctggtcaccgtctcctcag

SEQ ID NO: 255 - DNA, 337 bp, Canis sp.
gagatgcaactggtggagtctgggggagacctggtgaagcctggggggtccctgagactttcctgtgtggcctctggattcaccttcagtcgctaccacatgagctgggtccgccaggctccagggaaggggcttcagtgggtcgcatacattaatagtggtggaagtcccacaaactatgcagacgctgtgaagggccgattcaccatctccagagacaacgccaagaacacactatatcttcagatgaacagcctgagagccgaggacacggccgtgtattactgtacgacctacgaccagtggggccagggaaccctggtcaccgtctcctcag

SEQ ID NO: 256 - DNA, 337 bp, Canis sp.
gaggtgcacctggtggagtctgggggagacctggtgaagcctggggggtccctgagactttcctgtgtggcctctggaatcaccttcaatagatattatatgaactgggtccgccagactccagggaaggggcttcagtgggtcgcatacatagacagtggtggaagtcccacaacctatgcagacgctgtaaagggccgattcaccatctccagagacaacgccaagaacacgttgtatcttcagatgaacagcctgagagccgaggacacggccgtatattactgtacgagttttgactactggggccagggaaccctggtcaccgtctcctcag

SEQ ID NO: 257 - DNA, 337 bp, Canis sp.
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcattcattaacactggtagaagtagcacaaactatgcagacgctgtggcgggccgattcaccatctccagagacaacgccaagaacacgctgtatcttcagatgaacagcctgagagccgaggacacggccgtgtattactgttcgaccctcgactactggggccagggaaccctggtcaccgtctcctcag

SEQ ID NO: 258 - DNA, 337 bp, Canis sp.
gaagtacagctggtggagtctgggggagacctggtgaagcctggggggtccctgagactttcctgtgtggcctccggattcaccttcagtagatactacatgaactgggtccgccaggctccagggaaggggcttcagtgggtcgcattcattaccagtggtggaagtagcacaagctatggagacgctgtgaagggccgattcaccatctccagagacaacgccaagaacacgctgtatcttcagatgaacagcctgagagccgaggacacggccgtgtattactgtacgagcttcgactactggggccagggaaccctggtcaccgtctcctcag

SEQ ID NO: 259 - DNA, 337 bp, Canis sp.
gaggtgcagttggtggagtctgggggagacctggtgaagcctggggggtccctgagactttcctgtgtggcctctggattcaccttcagtaggtattacatgacctgggtccgccaggctccagggaaggggcttcagtgggtcgcatacattaacagtgatagaagaagcacaacctatgcagacgctgtgaagggccgattcaccatctccagagacaacgccaaaaacacgctgtatcttcagatgaacagcctgagagccgaggacacggccgtatattactgtacgacccttgaatattggggccagggaaccctggtcaccgtctcctcag

SEQ ID NO: 260 - DNA, 337 bp, Canis sp.
gaggtgcagttggtggagtctgggggagacctggtgaagccgggggggtccctgagactttcctgtgtggcctccggaatcaccttcagtagatatcacatgagctgggtccgccaggctccggggaaggggcttcagtgggtcgcattcattaacactggtagaagtagcacaaactatgcggacgctgtggcgggccgattcaccatctccagagacaacgccaagaacacgctgtatcttcaaatgaacggcctgacagtcgaggacacggccgtgtattactgttcgaccctcgactactggggccagggaaccctggtcaccgtctcctcag

SEQ ID NO: 261 - DNA, 337 bp, Canis sp.
gaagttcaactggtggagtctgggggagacctggtgaagcctggggggtccctgagactttcctgtgtggcctctggaatcaccttcagtaggtactacatgaactgggtccgccaggctccagggaaggggcttcagtgggtcgcatacattgacagtggtggaagtcccacaacctatgcagacgctgtgaagggccgattcaccatctccagagacaacgccaagaaagcgctgtatcttcagatgaacagcctgagagccgaggacacggccgtatattactgtacgagttttgactactggggccagggaaccctggtcaccgtctcctcag

SEQ ID NO: 262 - DNA, 337 bp, Canis sp.
gaggtgcagctggtggagtctgggggagacctggtgaagcctggggggtccctgagactttcctgtgtggcctctggaatcaccttcagtagatattatatgaactgggtccgccaggctccagggaaggggcttcagtgggtcgcatacataaacagtggtggaagtcccacaacctatgcagacgctgtaaagggccgattcaccatctccagagacaacgccaagaacacgttgtatcttcagatgaacagcctgagagccgaggacacggccgtgtattactgtacgagttttgactactggggccagggaaccctggtcaccgtctcctcag

SEQ ID NO: 263 - DNA, 337 bp, Canis sp.
gaggtgcaactggtggagtctgggggagacctggtgaagcctggggggtccctgagactttcctgtgtggcctctggactcaccttcagtaggtaccacatgagctggatccgccaggctccagggaaggggcttcagtgggtcgcatacattagcagtggtggaagtagcacaaactatgcaggctctgtgaagggccgattcaccgtctccagagacaacgccaagaacactctgtatcttcagatgaacagtctgagagccgaagacacggccgtctattactgtacgagtcttgactactggggccagggaaccctggtcaccgtctcctcag

SEQ ID NO: 264 - DNA, 337 bp, Canis sp.
gaggtgcagctggtggagtctgggggagacctggtgaagccgggggggtccctgagactttcctgtgtggcctctggactcaccttcagtagataccacatgagttgggtccgccaggctccagggaaggggcttcagtgggtcgcattcattaacagtggtagaagtgacacaacctatgcagacgctgtgaagggccgattcaccatctccagagacaacgccaagaacacgctgtatcttcagatgaacagcctgagagccgaggacacggccgtttattactgtacgacttttgactactggggccagggaaccctggtcaccgtctcctcag

SEQ ID NO: 265 - DNA, 337 bp, Canis sp.
gaggtgcagttggtggagtctgggggagacctggtgaagcctggggggtccctgagactttcctgtgtggcctctggattaaccttcagtagatattacatgaactgggtccgccaggctccagggaaggggcttcagtgggtcgcatatattgatagtggtggaagtcccacaaactatgcagacgctgtgaagggccgattcaccatctccagagacaacgccaagaacacactgtatcttcagatgaacagcctgagagccgaggacacggccgtatattattgttcgagttttgactactggggccagggaaccctggtcaccgtctcctcag

SEQ ID NO: 266 - DNA, 337 bp, Canis sp.
gaggtgcagctggtgcagtctgggggagacctggtgaagcctggggggtccctgagactttcctgtgtggcctctggatacaccttcactaggtatcacatgagttgggtccgccaggctccagggaaggggcttcagtgggtcgcattcattaatagtgatggaactggtataacctatggagacgctgtgaagggccgattcaccatctccagagacaatgccaagaacacgctgtatcttcagatgaacagcctgagagccgaggacacggccatctattactgtacgacttttgactcctggggccagggaaccctggtcaccgtctcctcag

SEQ ID NO: 267 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgtcagatgtgggacagcagtgctaatgtgttcggcggaggcacccatctgaccgtcctcg

SEQ ID NO: 268 - DNA, 319 bp, Canis sp.
tcctatgtgctgacacagctgccattcaaaaatgtgaccctgaagcagccggcccacatcacctgtgggggagacaacattggaagtaaaagtgttcactggtaccagcagaagctgggccaggcccctgtattgattatctattatgatagtagaaggccgacagggatccctgagcgattctccggcgccaactcggggaacacggccaccctgaccatcagcggggccctggccgaggacgaggctgactattactgccaggtgtgggacagtagtgttaatgtgttcggcggaggcacccatctgaccgtcctcg

SEQ ID NO: 269 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactaattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 270 - DNA, 319 bp, Canis sp.
tcctatgtgctgacacaggtgccatccaaaaatgtgaccctgaagcagccggcccacatcacctgtgggggagacaacattggaggtaaaagtgttcactggtaccagcagaagctgggccaggcccctctactgattatctattatgataccaggaggccgacagggatccctgagcgcttctccggcgccaactcggggaacacggccaccctgaccatcagcagggccctggccgaggacgaggctgactattactgccaggtgtgggacagcagtggtaatgtgttcggcggaggcacccatctgaccgtcatcg

SEQ ID NO: 271 - DNA, 319 bp, Canis sp.
tcctatgtgctgacacagctgccatccaaaagtgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacaccagtgctaatgtgttcggcggaggcacccatctgaccgtcctcg

SEQ ID NO: 272 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgataccagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 273 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 274 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgctagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 275 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgccaggtgtgggacagcagtgctaatgtgttcggcggaggcacccatctgaccgtcctcg

SEQ ID NO: 276 - DNA, 319 bp, Canis sp.
tcctatgtgctgacacagctgccatccaaaagtgtgaccctgaagcagccggcccacatcacctgtgggggagacaacattggaagtaaaagtgttcactggtaccagcagaagctgggccaggcccctatactgattatctattatgataacaacaggccgacagggatccctgagcgattctccggcgccaactcggggaacacggccaccctgaccatcagcggggccctggccgaggacgaggctgactattactgccacgtgtgggacagcagtgttaatgtgttcggcggaggcacccatctgaccgtcctcg

SEQ ID NO: 277 - DNA, 319 bp, Canis sp.
tcctatgtcctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactatttctgccaggtgtgggacagcagtgctaatgtgttcggcggaggcacccatctgaccgtcctcg

SEQ ID NO: 278 - DNA, 319 bp, Canis sp.; identical to SEQ ID NO: 272

SEQ ID NO: 279 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagtaggaggccgacagggatccctgagcgattctccggcgccaactcggggaacacggccaccctgaccatcagcggggccctggccgaggacgaggctgactattactgccaggtgtgggacagcagtgataatgtgttcggcggaggcacccatctgaccgtcctcg

SEQ ID NO: 280 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctgcactgattatctattatgatagcagcaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 281 - DNA, 319 bp, Canis sp.
tcctatgtggtgacacagctgccatccaatactgtgaccctgaagcagccggcccacatcacctgtgggggagacaacattggaagtaaaagtgttcactggtaccagcagaagctgggccaggcccctgtgctgattatctattatgatagcaggaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 282 - DNA, 319 bp, Canis sp.
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagcaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 283 - DNA, 319 bp, Canis sp.
agctacgtgctgacacagctgcctagcaagcaagtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 284 - DNA, 319 bp, Canis sp.
agctacgtgctgacacagctgcctagcaaggccgtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 285 - DNA, 319 bp, Canis sp.
agctacgtgctgacacagctgcctagcaaagaggtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 286 - DNA, 319 bp, Canis sp.
agctacgtgctgacacagctgcctagcaagaacgtggccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 287 - DNA, 319 bp, Canis sp.
agctacgtgctgacacagctgcctagcaagtccgtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 288 - DNA, 319 bp, Canis sp.
agctacgtgctgacacagctgcctagcaagaccgtgaccctgaagcagcctgcccacatcacctgcggcggcgacaacatcggcagcaagagcgtgcactggtatcagcagaagctgggccaagcccctgtgctgatcatctactacgacagcagaagacctaccggcatccctgagagattcagcggcgccaacagcggcaacaccgccaccctgaccatcagcggcgccctggccgaggacgaggccgactactactgccaagtgtgggacagcagcgccaacgtgttcggcggaggcacccacctgaccgtgctgg

SEQ ID NO: 289 - DNA, 319 bp, Canis sp.
tcttacgtgctgacacagctgccatctaagtccgtgaccctgaagcagccagctcacatcacatgcggaggcgacaacatcggcagcaagtctgtgcattggtatcagcagaagctgggccaggctcccgtgctgatcatctactatgattccaggcggcccaccggaatccctgagaggttctccggcgccaacagcggcaataccgctacactgaccatcaacggcgccctggctgaggacgaggctgattactattgtcagatgtgggactcctccgccaacgtgttcggaggaggaacacacctgaccgtgctgg

SEQ ID NO: 290 - DNA, 319 bp, Canis sp.
tcctacgtgctgacacagctgccattcaagagcgtgaccctgaagcagccagctcacatcacatgcggaggcgacaacatcggctctaagtccgtgcattggtatcagcagaagctgggccaggctcccgtgctgatcatctactatgattctaggcggcccaccggcatccctgagaggttcagcggcgccaactctggcaataccgctacactgaccatctccggcgccctggctgaggacgaggctgattactattgtcaagtgtgggactccagcgtgaacgtgttcggcggcggcacacacctgaccgtgctgg

SEQ ID NO: 291 - DNA, 319 bp, Canis sp.
agctacgtgctgacacagctgccaagcaagtctgtgaccctgaagcagccagctcacatcacatgcggaggcgacaacatcggctccaagagcgtgcattggtatcagcagaagctgggccaggctcccgtgctgatcatctactatgattccagcaggccaaccggaatccctgagcggttctctggcgccaactccggcaataccgctacactgaccatctctggcgccctggctgaggacgaggctgattactattgtcaagtgtgggactcttccgctaacgtgttcggcggcggcacacacctgaccgtgctgg

SEQ ID NO: 292 - DNA, 319 bp, Canis sp.
tcctacgtgctgacacaggtgccatctaagtccgtgaccctgaagcagccagctcacatcacatgcggaggcgacaacatcggaggcaagagcgtgcattggtatcagcagaagctgggccaggctcccctgctgatcatctactatgatacaaggcggcccaccggcatccctgagagattcagcggcgccaactctggcaataccgctacactgaccatctctcgcgccctggctgaggacgaggctgattactattgtcaagtgtgggactccagcggaaacgtgttcggaggaggaacacacctgaccgtgatcg

SEQ ID NO: 293 - DNA, 319 bp, Canis sp.
tcctacgtgctgacacagctgccatctaagtccgtgaccctgaagcagccagctcacatcacatgcggaggcgacaacatcggcagcaagtctgtgcattggtatcagcagaagctgggccaggctcccatcctgatcatctactatgatacatctaggccaaccggaatccctgagcggttctccggagctaacagcggcaataccgctacactgaccatctccggcgccctggctgaggacgaggctgattactattgtcaagtgtgggactcctccgccaacgtgttcggaggaggaacacacctgaccgtgctgg

SEQ ID NO: 294 - DNA, 319 bp, Canis sp.; identical to SEQ ID NO: 291

SEQ ID NO: 295 - DNA, 319 bp, Canis sp.
agctacgtgctgacacagctgccaagcaagtctgtgaccctgaagcagccagctcacatcacatgcggaggcgacaacatcggctccaagagcgtgcattggtatcagcagaagctgggacaggctcccgtgctgatcatctactatgcttccagcaggccaaccggaatccctgagcggttctctggcgccaactccggcaataccgctacactgaccatctctggcgccctggctgaggacgaggctgattactattgtcaagtgtgggattcttccgctaacgtgttcggcggcggcacacacctgaccgtgctgg

SEQ ID NO: 296 - DNA, 319 bp, Canis sp.
tcttacgtgctgacacagctgccatctaagtccgtgaccctgaagcagccagctcacatcacatgcggaggcgacaacatcggcagcaagtctgtgcattggtatcagcagaagctgggccaggctcccgtgctgatcatctactatgattccaggcggcccaccggaatccctgagaggttctccggcgccaacagcggcaataccgctacactgaccatcaacggcgccctggctgaggacgaggctgattactattgtcaagtgtgggactcctccgccaacgtgttcggaggaggaacacacctgaccgtgctgg

SEQ ID NO: 297 - DNA, 319 bp, Canis sp.
agctacgtgctgacacagctgccaagcaagtctgtgaccctgaagcagccagctcacatcacatgcggaggcgacaacatcggctccaagagcgtgcattggtatcagcagaagctgggccaggctcccgtgctgatcatctactatgattccagcaggccaaccggaatccctgagcggttctctggcgccaactccggcaataccgctacactgaccatctctggcgccctggctgaggacgaggctgattacttctgtcaagtgtgggactcttccgctaacgtgttcggcggcggcacacacctgaccgtgctgg

SEQ ID NO: 298 - DNA, 319 bp, Canis sp.; identical to SEQ ID NO: 293

SEQ ID NO: 299 - DNA, 319 bp, Canis sp.
tcctacgtgctgacacagctgccatctaagtccgtgaccctgaagcagccagctcacatcacatgcggaggcgacaacatcggcagcaagtctgtgcattggtatcagcagaagctgggccaggctcccgtgctgatcatctactatgattctaggcggcccaccggcatccctgagaggttctccggcgccaacagcggcaataccgctacactgaccatctccggcgccctggctgaggacgaggctgattactattgtcaagtgtgggactccagcgataacgtgttcggcggcggcacacacctgaccgtgctgg

SEQ ID NO: 300 - DNA, 319 bp, Canis sp.
agctacgtgctgacacagctgccaagcaagtctgtgaccctgaagcagccagctcacatcacatgcggaggcgacaacatcggctccaagagcgtgcattggtatcagcagaagctgggacaggctcccgctctgatcatctactatgattccagcaggccaaccggaatccctgagcggttctctggcgccaactccggcaataccgctacactgaccatctctggcgccctggctgaggacgaggctgattactattgtcaagtgtgggactcttccgctaacgtgttcggcggcggcacacacctgaccgtgctgg

SEQ ID NO: 301 - DNA, 319 bp, Canis sp.
tcctacgtgctgacacagctgccatctaagtccgtgaccctgaagcagccagctcacatcacatgcggaggcgacaacatcggcagcaagtctgtgcattggtatcagcagaagctgggccaggctcccatcctgatcatctactatgattctaggcggcccaccggcatccctgagaggttctccggcgccaacagcggcaataccgctacactgaccatctccggcgccctggctgaggacgaggctgattactattgtcaagtgtgggactccagcggaaacgtgttcggaggaggaacacacctgaccgtgctgg

SEQ ID NO: 302 - protein, 19 aa, Canis sp.
ITISHFFKMENLNLIKAPM

SEQ ID NO: 303 - protein, 10 aa, Canis sp.
ENLNLIKAPM

SEQ ID NO: 304 - protein, 9 aa, Canis sp.
HTDVYAPIC

SEQ ID NO: 305 - protein, 50 aa, Canis sp.
NITISHFFKMENLNLIKAPMPYVDIHNCDPANPSEKNSLSIQYCGSIRSV

SEQ ID NO: 306 - protein, 9 aa, Homo sp.
PAGIYAPIC

SEQ ID NO: 307 - protein, 50 aa, Homo sp.
NIKISHFLKMESLNFIRAHTPYINIYNCEPANPSEKNSPSTQYCYSIQSL

SEQ ID NO: 308 - protein, 9 aa, Homo sp.
PAGIYAPIC

SEQ ID NO: 309 - protein, 9 aa, Canis sp.
HTDVYAPIC

SEQ ID NO: 310 - protein, 50 aa, Artificial Sequence
  DH03
NIKISHFLKMENLNLIKAPMPYVDIHNCDPANPSEKNSLSIQYCGSIRSV

SEQ ID NO: 311 - protein, 50 aa, Artificial Sequence
  DH04
NITISHFFKMESLNFIKAPMPYVDIHNCDPANPSEKNSLSIQYCGSIRSV

SEQ ID NO: 312 - protein, 50 aa, Artificial Sequence
  DH05
NITISHFFKMENLNLIRAHTPYVDIHNCDPANPSEKNSLSIQYCGSIRSV

SEQ ID NO: 313 - protein, 50 aa, Artificial Sequence
  DH06
NITISHFFKMENLNLIKAPMPYINIYNCDPANPSEKNSLSIQYCGSIRSV

SEQ ID NO: 314 - protein, 50 aa, Artificial Sequence
  DH07
NITISHFFKMENLNLIKAPMPYVDIHNCEPANPSEKNSLSIQYCGSIRSV

SEQ ID NO: 315 - protein, 50 aa, Artificial Sequence
  DH08
NITISHFFKMENLNLIKAPMPYVDIHNCDPANPSEKNSPSTQYCGSIRSV

SEQ ID NO: 316 - protein, 50 aa, Artificial Sequence
  DH09
NITISHFFKMENLNLIKAPMPYVDIHNCDPANPSEKNSLSIQYCYSIQSL

SEQ ID NO: 317 - protein, 9 aa, Canis sp.
HTDVYAPIC

SEQ ID NO: 318 - protein, 50 aa, Canis sp.; identical to SEQ ID NO: 305

SEQ ID NO: 319 - protein, 9 aa, Mus musculus
PTGVFAPIC

SEQ ID NO: 320 - protein, 50 aa, Mus musculus
NMTLSHFLKMRRLELIQTSKPYVDIYDCEPSNSSEKNSPSTQYCNSIQSV

SEQ ID NO: 321 - protein, 9 aa, Artificial Sequence
  DM01
PTGVYAPIC

SEQ ID NO: 322 - protein, 9 aa, Artificial Sequence
  DM02
HTDVFAPIC

SEQ ID NO: 323 - protein, 50 aa, Artificial Sequence
  DM03
NMTLSHFLKMENLNLIKAPMPYVDIHNCDPANPSEKNSLSIQYCGSIRSV

SEQ ID NO: 324 - protein, 50 aa, Artificial Sequence
  DM04
NITISHFFKMRRLELIKAPMPYVDIHNCDPANPSEKNSLSIQYCGSIRSV

SEQ ID NO: 325 - protein, 50 aa, Artificial Sequence
  DM05
NITISHFFKMENLNLIQTSKPYVDIHNCDPANPSEKNSLSIQYCGSIRSV

SEQ ID NO: 326 - protein, 50 aa, Artificial Sequence
  DM06
NITISHFFKMENLNLIKAPMPYVDIYDCDPANPSEKNSLSIQYCGSIRSV

SEQ ID NO: 327 - protein, 50 aa, Artificial Sequence
  DM07
NITISHFFKMENLNLIKAPMPYVDIHNCEPSNSSEKNSLSIQYCGSIRSV

SEQ ID NO: 328 - protein, 50 aa, Artificial Sequence; identical to SEQ ID NO: 315
  DM08

SEQ ID NO: 329 - protein, 50 aa, Artificial Sequence
  DM09
NITISHFFKMENLNLIKAPMPYVDIHNCDPANPSEKNSLSIQYCNSIQSV

SEQ ID NO: 330 - protein, 9 aa, Canis sp.
HTDVYAPIC

SEQ ID NO: 331 - protein, 9 aa, Artificial Sequence
  S01
ATDVYAPIC

SEQ ID NO: 332 - protein, 9 aa, Artificial Sequence
  S02
HADVYAPIC

SEQ ID NO: 333 - protein, 9 aa, Artificial Sequence
  S03
HTAVYAPIC

SEQ ID NO: 334 - protein, 9 aa, Artificial Sequence
  S04
HTDAYAPIC

SEQ ID NO: 335 - protein, 9 aa, Artificial Sequence
  S05
HTDVAAPIC

SEQ ID NO: 336 - protein, 9 aa, Artificial Sequence
  S06
HTDVYAPIC

SEQ ID NO: 337 - protein, 9 aa, Artificial Sequence
  S07
HTDVYAAIC

SEQ ID NO: 338 - protein, 9 aa, Artificial Sequence
  S08
HTDVYAPAC

SEQ ID NO: 339 - protein, 9 aa, Artificial Sequence
  S09
HTDVYAPIA

SEQ ID NO: 340 - protein, 50 aa, Canis sp.; identical to SEQ ID NO: 305

SEQ ID NO: 341 - protein, 50 aa, Artificial Sequence
  L01
AITISHFFKMENLNLIKAPMPYVDIHNCDPANPSEKNSLSIQYCGSIRSV

SEQ ID NO: 342 - protein, 50 aa, Artificial Sequence
  L02
NIAISHFFKMENLNLIKAPMPYVDIHNCDPANPSEKNSLSIQYCGSIRSV

SEQ ID NO: 343 - protein, 50 aa, Artificial Sequence
  L03
NITIAHFFKMENLNLIKAPMPYVDIHNCDPANPSEKNSLSIQYCGSIRSV

SEQ ID NO: 344 - protein, 50 aa, Artificial Sequence
  L04
NITISHAFKMENLNLIKAPMPYVDIHNCDPANPSEKNSLSIQYCGSIRSV

SEQ ID NO: 345 - protein, 50 aa, Artificial Sequence
  L05
NITISHFFAMENLNLIKAPMPYVDIHNCDPANPSEKNSLSIQYCGSIRSV

SEQ ID NO: 346 - protein, 50 aa, Artificial Sequence
  L06
NITISHFFKMANLNLIKAPMPYVDIHNCDPANPSEKNSLSIQYCGSIRSV

SEQ ID NO: 347 - protein, 50 aa, Artificial Sequence
  L07
NITISHFFKMENANLIKAPMPYVDIHNCDPANPSEKNSLSIQYCGSIRSV

SEQ ID NO: 348 - protein, 50 aa, Artificial Sequence
  L08
NITISHFFKMENLNAIKAPMPYVDIHNCDPANPSEKNSLSIQYCGSIRSV

SEQ ID NO: 349 - protein, 50 aa, Artificial Sequence
  L09
NITISHFFKMENLNLIAAPMPYVDIHNCDPANPSEKNSLSIQYCGSIRSV

SEQ ID NO: 350 - protein, 50 aa, Artificial Sequence
  L10
NITISHFFKMENLNLIKAAMPYVDIHNCDPANPSEKNSLSIQYCGSIRSV

SEQ ID NO: 351 - protein, 50 aa, Artificial Sequence
  L11
NITISHFFKMENLNLIKAPMAYVDIHNCDPANPSEKNSLSIQYCGSIRSV

SEQ ID NO: 352 - protein, 50 aa, Artificial Sequence
  L12
NITISHFFKMENLNLIKAPMPYADIHNCDPANPSEKNSLSIQYCGSIRSV

SEQ ID NO: 353 - protein, 50 aa, Artificial Sequence
  L13
NITISHFFKMENLNLIKAPMPYVDAHNCDPANPSEKNSLSIQYCGSIRSV

SEQ ID NO: 354 - protein, 50 aa, Artificial Sequence
  L14
NITISHFFKMENLNLIKAPMPYVDIHACDPANPSEKNSLSIQYCGSIRSV

SEQ ID NO: 355 - protein, 50 aa, Artificial Sequence
  L15
NITISHFFKMENLNLIKAPMPYVDIHNCAPANPSEKNSLSIQYCGSIRSV

SEQ ID NO: 356 - protein, 50 aa, Artificial Sequence; identical to SEQ ID NO: 305
  L16

SEQ ID NO: 357 - protein, 50 aa, Artificial Sequence
  L17
NITISHFFKMENLNLIKAPMPYVDIHNCDPANASEKNSLSIQYCGSIRSV

SEQ ID NO: 358 - protein, 50 aa, Artificial Sequence
  L18
NITISHFFKMENLNLIKAPMPYVDIHNCDPANPSAKNSLSIQYCGSIRSV

SEQ ID NO: 359 - protein, 50 aa, Artificial Sequence
  L19
NITISHFFKMENLNLIKAPMPYVDIHNCDPANPSEKASLSIQYCGSIRSV

SEQ ID NO: 360 - protein, 50 aa, Artificial Sequence
  L20
NITISHFFKMENLNLIKAPMPYVDIHNCDPANPSEKNSASIQYCGSIRSV

SEQ ID NO: 361 - protein, 50 aa, Artificial Sequence
  L21
NITISHFFKMENLNLIKAPMPYVDIHNCDPANPSEKNSLSAQYCGSIRSV

SEQ ID NO: 362 - protein, 50 aa, Artificial Sequence
  L22
NITISHFFKMENLNLIKAPMPYVDIHNCDPANPSEKNSLSIQACGSIRSV

SEQ ID NO: 363 - protein, 50 aa, Artificial Sequence
  L23
NITISHFFKMENLNLIKAPMPYVDIHNCDPANPSEKNSLSIQYCASIRSV

SEQ ID NO: 364 - protein, 50 aa, Artificial Sequence
  L24
NITISHFFKMENLNLIKAPMPYVDIHNCDPANPSEKNSLSIQYCGSARSV

SEQ ID NO: 365 - protein, 50 aa, Artificial Sequence
  L25
NITISHFFKMENLNLIKAPMPYVDIHNCDPANPSEKNSLSIQYCGSIRAV